SEQUENCE LISTING (27 sequences)

SEQ ID NO: 1 - RNA, 7310 nt, Senecavirus A
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 2 - RNA, 7310 nt, Artificial Sequence
  Senecavirus A S177A mutant
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucaggcgcuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 3 - RNA, 7312 nt, Artificial Sequence
  Senecavirus A IR2 mutant
uuugaaauggggggcugggcccugaugcccaguccuuccuuuccccuuccgggggguuaaccggcuguguuugcuagaggcacagaggagcaacauccaaccugcuuuuguggggaacggugcggcuccaauuccugcgucgccaaagguguuagcgcacccaaacggcgcaucuaccaaugcuauugguguggucugcgaguucuagccuacucguuucuccccuauucauucacucacgcacaaaaaguguguuguaacuacaagauuuagcccucacacgggaugugugauaaccgcaagacugacucaagcgcggaaagcgcuguaaccgcaugcuguuagucccuuuauggcugcgagauggcuauccaccucggaucacugaacuggagcucgacccuccuuaguaagggaaccgagaggccuucuugcaacaagcuccgacacagaguccacgugauugcuaccaccaugaguacaugguucuccccucucgacccaggacuucuuuuugaauauccacggcucgauccagaggguggggcaugaucccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuccuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuccuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 4 - RNA, 7313 nt, Artificial Sequence
  Senecavirus A S177A IR2 mutant
uuugaaauggggggcugggcccugaugcccaguccuuccuuuccccuuccgggggguuaaccggcuguguuugcuagaggcacagaggagcaacauccaaccugcuuuuguggggaacggugcggcuccaauuccugcgucgccaaagguguuagcgcacccaaacggcgcaucuaccaaugcuauugguguggucugcgaguucuagccuacucguuucuccccuauucauucacucacgcacaaaaaguguguuguaacuacaagauuuagcccucacacgggaugugugauaaccgcaagacugacucaagcgcggaaagcgcuguaaccgcaugcuguuagucccuuuauggcugcgagauggcuauccaccucggaucacugaacuggagcucgacccuccuuaguaagggaaccgagaggccuucuugcaacaagcuccgacacagaguccacgugauugcuaccaccaugaguacaugguucuccccucucgacccaggacuucuuuuugaauauccacggcucgauccagaggguggggcaugaucccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuccuuguugccaugguuccugaaaccacccuugaugucaagcccgacgguaaggcaaagagcuuacaggagcugaaugaagaacaguggguggaaaugucugacgauuaccggaccgggaaaaacaugccuuuucaggcgcuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuccuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaaa

SEQ ID NO: 5 - RNA, 8066 nt, Artificial Sequence
  SVV-mCherry construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaaggagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 6 - RNA, 7992 nt, Artificial Sequence
  SVV-nLuc construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcggagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaaggccggcauggucccagccuccucgcuggcgccggcugggcaacaugcuucggcauggcgaaugggacgcggccgcucgagucuagagggcccguuuaaacccgcugaucagccu

SEQ ID NO: 7 - RNA, 7786 nt, Artificial Sequence
  SVV-GMCSF construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgauguggcuccaaaaccugcucuuccucggaauugucguguacucccuguccgcuccuacuagaagcccaaucacaguaacaagaccuuggaaacacguggaagcuauaaaagaagcccugaaccugcuugacgauaugcccguaacccugaaugaagaaguugaaguuguaagcaacgaauuuuccuucaagaaacucacaugugugcaaacccggcugaaaauauuugaacaaggacugagaggaaauuuuacuaaacuuaaaggcgcacuuaauaugacugcuucuuauuaccagacuuauugccccccuacaccagaaaccgauugcgaaacccaagugacuaccuaugccgacuuuaucgauuccuugaaaacguuccuuacugauauacccuuugaaugcaaaaaaccuggacagaaagagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaa

SEQ ID NO: 8 - RNA, 8993 nt, Artificial Sequence
  SVV IL-12 construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgauguguccucagaagcuaaccaucuccugguuugccaucguuuugcuggugucuccacucauggccaugugggagcuggagaaagacguuuauguuguagagguggacuggacucccgaugccccuggagaaacagugaaccucaccugugacacgccugaagaagaugacaucaccuggaccucagaccagagacauggagucauaggcucuggaaagacccugaccaucacugucaaagaguuucuagaugcuggccaguacaccugccacaaaggaggcgagacucugagccacucacaucugcugcuccacaagaaggaaaauggaauuugguccacugaaauuuuaaaaaauuucaaaaacaagacuuuccugaagugugaagcaccaaauuacuccggacgguucacgugcucauggcuggugcaaagaaacauggacuugaaguucaacaucaagagcaguagcaguuccccugacucucgggcagugacauguggaauggcgucucugucugcagagaaggucacacuggaccaaagggacuaugagaaguauucaguguccugccaggaggaugucaccugcccaacugccgaggagacccugcccauugaacuggcguuggaagcacggcagcagaauaaauaugagaacuacagcaccagcuucuucaucagggacaucaucaaaccagacccgcccaagaacuugcagaugaagccuuugaagaacucacagguggaggucagcugggaguacccugacuccuggagcacuccccauuccuacuucucccucaaguucuuuguucgaauccagcgcaagaaagaaaagaugaaggagacagaggagggguguaaccagaaaggugcguuccucguagagaagacaucuaccgaaguccaaugcaaaggcgggaaugucugcgugcaagcucaggaucgcuauuacaauuccucgugcagcaagugggcauguguucccugcaggguccgauccggagguggcgguucuggcgguggagggagcggaggcggaggaucaagggucauuccagucucuggaccugccaggugucuuagccagucccgaaaccugcugaagaccacagaugacauggugaagacggccagagaaaaacugaaacauuauuccugcacugcugaagacaucgaucaugaagacaucacacgggaccaaaccagcacauugaagaccuguuuaccacuggaacuacacaagaacgagaguugccuggcuacuagagagacuucuuccacaacaagagggagcugccugcccccacagaagacgucuuugaugaugacccugugccuugguagcaucuaugaggacuugaagauguaccagacagaguuccaggccaucaacgcagcacuucagaaucacaaccaucagcagaucauucuagacaagggcaugcugguggccaucgaugagcugaugcagucucugaaucauaauggcgagacucugcgccagaaaccuccugugggagaagcagacccuuacagagugaaaaugaagcucugcauccugcuucacgccuucagcacccgcgucgugaccaucaacagggugaugggcuaucugagcuccgccgagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 9 - RNA, 8905 nt, Artificial Sequence
  SVV mFAP-CD3 BiTE construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgaugguuuugcuugugaccagccuccugcucugugaacugccucauccugcauuccuguugaucccacaggugcagcuccagcagaguggcgcagagcucgcucgcccaggcgcuucugugaaucugaguuguaaggccuccggauauacuuuuacgaacaacggcaucaacuggcugaagcagcggaccggccagggccuggaguggaucggcgaaauauacccccgguccacaaacacucucuauaacgagaaguuuaagggcaaagcaacucugaccgcggacagguccucuaacacagccuauauggagcugagaagcuugacgagugaggacuccgcugucuauuuuugcgcccgaacucugaccgcuccuuuugcuuuuuggggccagggcacgcucgugaccguaagugcgggcuccacuagcgguuccggcaaaccuggcagcggagaaggcagcaccaaagggcagaucguccugacgcagucuccagccaucaugagcgccucacccggcgaaaaggugaccaugaccugcucagccucuucuggugugaauuucaugcacugguaccagcaaaaaagugggaccuccccuaaaagguggaucuucgauaccagcaaacuggcuucuggcguucccgcaagguuuagcggcucugguuccggcacaucauacagccugacgaucagcagcauggaggcagaagacgcagcuaccuauuacugccagcaauggagcuuuaacccaccuacuuucggaggaggaacaaagcuggaaauaaaaagagguggugguggaucagaggugcagcugguggagucugggggaggcuuggugcagccuggaaagucccugaaacucuccugugaggccucuggauucaccuucagcggcuauggcaugcacuggguccgccaggcuccagggagggggcuggagucggucgcauacauuacuaguaguaguauuaauaucaaauaugcugacgcugugaaaggccgguucaccgucuccagagacaaugccaagaacuuacuguuucuacaaaugaacauucucaagucugaggacacagccauguacuacugugcaagauucgacugggacaaaaauuacuggggccaaggaaccauggucaccgucuccucagguggugguggaucagguggaggcggaaguggagguggcggauccgacauccagaugacccagucuccaucaucacugccugccucccugggagacagagucacuaucaauugucaggccagucaggacauuagcaauuauuuaaacugguaccagcagaaaccagggaaagcuccuaagcuccugaucuauuauacaaauaaauuggcagauggagucccaucaagguucaguggcagugguucugggagagauucuucuuucacuaucagcagccuggaauccgaagauauuggaucuuauuacugucaacaguauuauaacuauccguggacguucggaccuggcaccaagcuggaaaucaaacggcaccaccaucaucaccacgagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuaacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 10 - RNA, 7658 nt, Artificial Sequence
  SVV CXCL10 construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgaugaacccaagugcugccgucauuuucugccucauccugcugggucugagugggacucaagggaucccucucgcaaggacgguccgcugcaacugcauccauaucgaugacgggccagugagaaugagggccauagggaagcuugaaaucaucccugcgagccuauccugcccacguguugagaucauugccacgaugaaaaagaaugaugagcagagaugucugaauccggaaucuaagaccaucaagaauuuaaugaaagcguuuagccaaaaaaggucuaaaagggcuccugagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 11 - RNA, 7871 nt, Artificial Sequence
  SVV-hIL36 construct
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccgaugagaggcacuccaggagacgcugaugguggaggaagggccgucuaucaaucaauguguaaaccuauuacugggacuauuaaugauuugaaucagcaaguguggacccuucagggucagaaccuuguggcaguuccacgaagugacagugugaccccagucacuguugcuguuaucacaugcaaguauccagaggcucuugagcaaggcagaggggaucccauuuauuugggaauccagaauccagaaauguguuuguauugugagaagguuggagaacagcccacauugcagcuaaaagagcagaagaucauggaucuguauggccaacccgagcccgugaaacccuuccuuuucuaccgugccaagacugguaggaccuccacccuugagucuguggccuucccggacugguucauugccuccuccaagagagaccagcccaucauucugacuucagaacuugggaagucauacaacacugccuuugaauuaaauauaaaugacgagggcagaggaagucugcuaacaugcggugacgucgaggagaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 12 - DNA, 7540 bp, Artificial Sequence
  SVV IVT template - 5′ HHR and 3′ HDV
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 13 - DNA, 7540 bp, Artificial Sequence
  SVV-177A mutant IVT template - 5′ HHR and 3′ HDV
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 14 - DNA, 7455 bp, Artificial Sequence
  SVV-WT IVT template - 5′ Pistol 1 and 3′ SapI
ttatcgaaattaatacgactcactatagggcgtcatagtaggaagtattgggttgggaagaacttatcgtggactcgtctgagcgagtataaacagtcaattaagctcagagcgttcaccggggaaattcggtgaggtttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaagaagagc

SEQ ID NO: 15 - DNA, 7455 bp, Artificial Sequence; identical to SEQ ID NO: 14
  SVV Pistol2-SapI IVT template

SEQ ID NO: 16 - DNA, 7457 bp, Artificial Sequence
  SVV-IR2-Pistol1-SapI IVT Template
ttatcgaaattaatacgactcactatagggcgtcatagtaggaagtattgggttgggaagaacttatcgtggactcgtctgagcgagtataaacagtcaattaagctcagagcgttcaccggggaaattcggtgaggtttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaagaagagc

SEQ ID NO: 17 - DNA, 7457 bp, Artificial Sequence
  SVV-IR2-S177A-Pistol1-SapI IVT template
ttatcgaaattaatacgactcactatagggcgtcatagtaggaagtattgggttgggaagaacttatcgtggactcgtctgagcgagtataaacagtcaattaagctcagagcgttcaccggggaaattcggtgaggtttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaagaagagc

SEQ ID NO: 18 - DNA, 7445 bp, Artificial Sequence
  SVV-HHRS-SapI IVT Template
ttatcgaaattaatacgactcactatagggcgtcatagtaggaagtattgggttgggaagaacttatcgttggtaccttaccatttcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaagaagagc

SEQ ID NO: 19 - DNA, 8296 bp, Artificial Sequence
  SVV-mCherry IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 20 - DNA, 8107 bp, Artificial Sequence
  SVV-nLuc IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 21 - DNA, 8017 bp, Artificial Sequence
  SVV-GMCSF IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgtggctccaaaacctgctcttcctcggaattgtcgtgtactccctgtccgctcctactagaagcccaatcacagtaacaagaccttggaaacacgtggaagctataaaagaagccctgaacctgcttgacgatatgcccgtaaccctgaatgaagaagttgaagttgtaagcaacgaattttccttcaagaaactcacatgtgtgcaaacccggctgaaaatatttgaacaaggactgagaggaaattttactaaacttaaaggcgcacttaatatgactgcttcttattaccagacttattgcccccctacaccagaaaccgattgcgaaacccaagtgactacctatgccgactttatcgattccttgaaaacgttccttactgatataccctttgaatgcaaaaaacctggacagaaagagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 22 - DNA, 9258 bp, Artificial Sequence
  SVV-mIL-12 IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggtggcggttctggcggtggagggagcggaggcggaggatcaagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgcggccgctcgagtctagagggcccgtttaaacccgctgatcagcct

SEQ ID NO: 23 - DNA, 9135 bp, Artificial Sequence
  SVV-mFap-BiTE IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatggttttgcttgtgaccagcctcctgctctgtgaactgcctcatcctgcattcctgttgatcccacaggtgcagctccagcagagtggcgcagagctcgctcgcccaggcgcttctgtgaatctgagttgtaaggcctccggatatacttttacgaacaacggcatcaactggctgaagcagcggaccggccagggcctggagtggatcggcgaaatatacccccggtccacaaacactctctataacgagaagtttaagggcaaagcaactctgaccgcggacaggtcctctaacacagcctatatggagctgagaagcttgacgagtgaggactccgctgtctatttttgcgcccgaactctgaccgctccttttgctttttggggccagggcacgctcgtgaccgtaagtgcgggctccactagcggttccggcaaacctggcagcggagaaggcagcaccaaagggcagatcgtcctgacgcagtctccagccatcatgagcgcctcacccggcgaaaaggtgaccatgacctgctcagcctcttctggtgtgaatttcatgcactggtaccagcaaaaaagtgggacctcccctaaaaggtggatcttcgataccagcaaactggcttctggcgttcccgcaaggtttagcggctctggttccggcacatcatacagcctgacgatcagcagcatggaggcagaagacgcagctacctattactgccagcaatggagctttaacccacctactttcggaggaggaacaaagctggaaataaaaagaggtggtggtggatcagaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggtggtggatcaggtggaggcggaagtggaggtggcggatccgacatccagatgacccagtctccatcatcactgcctgcctccctgggagacagagtcactatcaattgtcaggccagtcaggacattagcaattatttaaactggtaccagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaacggcaccaccatcatcaccacgagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattaactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 24 - DNA, 7888 bp, Artificial Sequence
  SVV-CXCL10 IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctcctgagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 25 - DNA, 8101 bp, Artificial Sequence
  SVV-hIL36 IVT template
ttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgatgagaggcactccaggagacgctgatggtggaggaagggccgtctatcaatcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgacgagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcct

SEQ ID NO: 26 - DNA, 713 bp, Artificial Sequence
  Modified CVA21 5′ UTR
ttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaa

SEQ ID NO: 27 - DNA, 7435 bp, Artificial Sequence
  Modified CVA21
ttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagacctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggagaaaaaaaaaaaaaaaaaaaaaaaaaaaaaa

(19) United States
(12) Patent Application Publication
(10) Pub. No.: US 2022/0117902 A1
(43) Pub. Date: Apr. 21, 2022

(54) ENCAPSULATED RNA POLYNUCLEOTIDES AND METHODS OF USE

(71) Applicant: ONCORUS, INC., Cambridge, MA (US)

(72) Inventors: Lorena LERNER, Cambridge, MA (US); Edward M. KENNEDY, Cambridge, MA (US); Mitchell H. FINER, Cambridge, MA (US); Christophe QUÉVA, Cambridge, MA (US)

(21) Appl. No.: 17/418,465

(22) PCT Filed: Jan. 3, 2020
(86) PCT No.: PCT/US2020/012237

§ 371 Date: Jun. 25, 2021

Related U.S. Application Data
(60) Provisional application No. 62/788,504, filed on Jan. 4, 2019.
(60) Provisional application No. 62/895,135, filed on Sep. 3, 2019.

(51) Int. Cl. A61K 9/51; C12N 7/00; A61K 35/768; A61P 35/00

(52) U.S. Cl. CPC A61K 9/5123; C12N 7/00; A61K 35/768; C12N 2770/32051; A61P 35/00; C12N 2770/32021; C12N 2770/32032; A61K 9/5115

(57) ABSTRACT

The present disclosure relates to recombinant RNA molecules encoding an oncolytic virus. The present disclosure further relates to the encapsulation of the recombinant RNA molecules and the use of the recombinant RNA molecules and/or particles for the treatment and prevention of cancer.

### CROSS-REFERENCE TO RELATED APPLICATIONS

This application claims priority to U.S. Provisional Application No. 62/788,504, filed Jan. 4, 2019 and 62/895,135, filed Sep. 3, 2019, the contents of which are each incorporated herein by reference in their entireties.

### DESCRIPTION OF THE TEXT FILE SUBMITTED ELECTRONICALLY

The contents of the text file submitted electronically herewith are incorporated herein by reference in their entirety. A computer readable format copy of the Sequence Listing (filename: ONCR-015_01WO_SeqList_ST25.txt, date recorded: Jan. 2, 2020, file size: 265 kilobytes).

### FIELD

The present disclosure generally relates to the fields of immunology, inflammation, and cancer therapeutics. More specifically, the present disclosure relates to particle-encapsulated viral genomes. The disclosure further relates to the treatment and prevention of proliferative disorders such as cancer.

### BACKGROUND

Oncolytic viruses are replication-competent viruses with lytic life-cycle able to infect and lyse tumor cells. Direct tumor cell lysis results not only in cell death, but also the generation of an adaptive immune response against tumor antigens taken up and presented by local antigen presenting cells. Therefore, oncolytic viruses combat tumor cell growth through both direct cell lysis and by promoting antigen-specific adaptive responses capable of maintaining anti-tumor responses after viral clearance.

However, clinical use of replication-competent viruses poses several challenges. In general, viral exposure activates innate immune pathways, resulting in a broad, non-specific inflammatory response. If the patient has not been previously exposed to the virus, this initial innate immune response can lead to the development of an adaptive anti-viral response and the production of neutralizing antibodies. If a patient has been previously exposed to the virus, existing neutralizing anti-viral antibodies can prevent the desired lytic effects. In both instances, the presence of neutralizing antibodies not only prevents viral lysis of target cells, but also renders re-administration of the viral therapeutic ineffective. These factors limit the use of viral therapeutics in the treatment of metastatic cancers, as the efficacy of repeated systemic administration required for treatment of such cancers is hampered by naturally-occurring anti-viral responses. Even in the absence of such obstacles, subsequent viral replication in non-diseased cells can result in substantial off-disease collateral damage to surrounding cells and tissues.

There remains a long-felt and unmet need in the art for compositions and methods related to therapeutic use of replication-competent virus. The present disclosure provides such compositions and methods, and more.

### SUMMARY

In some embodiments, the present disclosure provides a lipid nanoparticle (LNP) comprising a synthetic RNA viral genome encoding an oncolytic virus. In some embodiments, the oncolytic virus is a single-stranded RNA (ssRNA) virus. In some embodiments, the oncolytic virus is a positive sense ((+-sense) ssRNA virus. In some embodiments, the (+)-sense ssRNA virus is selected from those listed in Table 1. In some embodiments, the (+)-sense ssRNA virus is a Picornavirus. In some embodiments, the Picornavirus is a Seneca Valley Virus (SVV) or a Coxsackievirus. In some embodiments, the SVV is an SVV-A selected from a wild type SVV-A (SEQ ID NO: 1), an S177A-SVVA mutant (SEQ ID NO: 2), an SVV-IR2 mutant (SEQ ID NO: 3), and an SVV-IR2-S177A mutant (SEQ ID NO: 4). In some embodiments, the Coxsackievirus is selected from CVB3, CVA21, and CVA9. In some embodiments, the Coxsackievirus is a modified CVA21 virus comprising SEQ ID NO: 27.

In some embodiments, delivery of the LNP to a cell results in production of viral particles by the cell, and wherein the viral particles are infectious and lytic. In some embodiments, the encoded oncolytic virus is capable of reducing the size of a tumor that is remote from the site of LNP administration to a subject.

In some embodiments, the synthetic RNA viral genome further comprises a heterologous polynucleotide encoding an exogenous payload protein. In some embodiments, the LNP further comprises a recombinant RNA molecule encoding an exogenous payload protein. In some embodiments, the exogenous payload protein is a fluorescent protein, an enzymatic protein, a cytokine, a chemokine, an antigen-binding molecule capable of binding to a cell surface receptor, or a ligand for a cell-surface receptor. In some embodiments, the cytokine is selected from IL-12, GM-CSF, IL-18, IL-2, and IL-36γ. In some embodiments, the ligand for a cell-surface receptor is Flt3 ligand or TNFSF14. In some embodiments, the chemokine is selected from CXCL10, CCL4, CCL21, and CCL5. In some embodiments, the antigen-binding molecule is capable of binding to and inhibiting an immune checkpoint receptor. In some embodiments, the immune checkpoint receptor is PD-1. In some embodiments, the antigen-binding molecule is capable of binding to a tumor antigen. In some embodiments, the antigen binding molecule is a bispecific T cell engager molecule (BiTE) or a bispecific light T cell engager molecule (LiTE). In some embodiments, the tumor antigen is DLL3 or EpCAM.

In some embodiments, the synthetic RNA viral genome and/or the recombinant RNA molecule comprises a micro RNA (miRNA) target sequence (miR-TS) cassette, wherein the miR-TS cassette comprises one or more miRNA target sequences. In some embodiments, the one or more miRNAs are selected from miR-124, miR-1, miR-143, miR-128, miR-219, miR-219a, miR-122, miR-204, miR-217, miR-137, and miR-126. In some embodiments, the miR-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, the miR-TS cassette comprises one or more copies of a miR-217 target sequence, one or more copies of a miR-137 target sequence, and one or more copies of a miR-126 target sequence.

In some embodiments, the LNP comprises a cationic lipid, one or more helper lipids, and a phospholipid-polymer conjugate. In some embodiments, the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP). In some embodiments, the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol.

In some embodiments, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

In some embodiments, the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine). In some embodiments, n the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

In some embodiments, the cationic lipid comprises COATSOME® SS-OC, wherein the one or more helper lipids comprise cholesterol (Chol) and DSPC, and wherein the phospholipid-polymer conjugate comprises DPG-PEG2000. In some embodiments, the ratio of SS-OC:DSPC:Chol:DPG-PEG2K (as a percentage of total lipid content) is A:B:C:D, wherein A=40%-60%, B=10%-25%, C=20%-30%, and D=0%-3% and wherein A+B+C+D=100%. In some embodiments, the ratio of SS-OC:DSPC:Chol:DPG-PEG2K (as a percentage of total lipid content) is A:B:C:D, wherein A=45%-50%, B=20%-25%, C=25%-30%, and D=0%-1% and wherein A+B+C+D=100%. In some embodiments, the ratio of SS-OC:DSPC:Chol:DPG-PEG2K (as a percentage of total lipid content) is about 49:22:28.5:0.5.

In some embodiments, the ratio of SS-OC:DSPC:Chol:DPG-PEG2K (as a percentage of total lipid content) is A:B:C:D, wherein A=40%-60%, B=10%-30%, C=20%-45%, and D=0%-3% and wherein A+B+C+D=100%. In some embodiments, the ratio of SS-OC:DSPC:Chol:DPG-PEG2K (as a percentage of total lipid content) is A:B:C:D, wherein A=40%-60%, B=10%-30%, C=25%-45%, and D=0%-3% and wherein A+B+C+D=100%. In some embodiments, the ratio of SS-OC:DSPC:Chol:DPG-PEG2K (as a percentage of total lipid content) is A:B:C:D, wherein A=45%-55%, B=10%-20%, C=30%-40%, and D=1%-2% and wherein A+B+C+D=100%. In some embodiments, the ratio of SS-OC:DSPC:Chol:DPG-PEG2K (as a percentage of total lipid content) is A:B:C:D, wherein A=45%-50%, B=10%-15%, C=35%-40%, and D=1%-2% and wherein A+B+C+D=100%. In some embodiments, the ratio of SS-OC:DSPC:Chol:DPG-PEG2K (as a percentage of total lipid content) is 49:11:38.5:1.5.

In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-65%, B=5%-20%, C=20%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=50%-60%, B=5%-15%, C=30%-45%, and D=0%-3% and wherein A+B+C+D 100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=55%-60%, B 5%-15%, C 30%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=55%-60%, B=5%-10%, C=30%-35%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is 58:7:33.5:1.5.

In some embodiments, the LNP comprises a lipid formulation selected from Table 5.

In some embodiments, hyaluronan is conjugated to the surface of the LNP.

In some embodiments, the present disclosure provides a therapeutic composition comprising a plurality of lipid nanoparticles described herein. In some embodiments, the plurality of LNPs have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm to about 120 nm. In some embodiments, the plurality of LNPs have an average size of about 50 nm, 60 nm, 70 nm, 80 nm, 90 nm, 100 nm, 110 nm, or about 120 nm. In some embodiments, the plurality of LNPs have an average size of about 100 nm.

In some embodiments, the plurality of LNPs have an average zeta-potential of between about 40 mV to about −40 mV, about 20 mV to about −20 mV, about 10 mV to about −10 mV, about 5 mV to about −5 mV, or about 20 mV to about −40 mV. In some embodiments, the plurality of LNPs have an average zeta-potential of less than about −20 mV, less than about −30 mV, less than about 35 mV, or less than about −40 mV. In some embodiments, the plurality of LNPs have an average zeta-potential of between about −50 mV to about −20 mV, about −40 mV to about −20 mV, or about −30 mV to about −20 mV. In some embodiments, the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In some embodiments, administering the therapeutic composition to a subject delivers the recombinant RNA polynucleotide to a target cell of the subject, and wherein the recombinant RNA polynucleotide produces an infectious oncolytic virus capable of lysing the target cell of the subject. In some embodiments, the target cell is a cancerous cell.

In some embodiments, the composition is formulated for intravenous or intratumoral delivery.

In some embodiments, the present disclosure provides a method of inhibiting the growth of a cancerous tumor in a subject in need thereof comprising administering the therapeutic composition described herein to the subject in need thereof, wherein administration of the composition inhibits the growth of the tumor. In some embodiments, the administration is intratumoral or intravenous. In some embodiments, the cancer is a lung cancer, a liver cancer, a prostate cancer, a bladder cancer, or a melanoma.

In some embodiments, the present disclosure provides a recombinant RNA molecule comprising a synthetic RNA viral genome encoding an oncolytic virus. In some embodiments, the encoded oncolytic virus is a single-stranded RNA (ssRNA) virus. In some embodiments, the ssRNA virus is a positive sense ((+)-sense) or a negative-sense ((−)-sense) ssRNA virus. In some embodiments, the (+)-sense ssRNA virus is a Picornavirus. In some embodiments, the Picornavirus is a Seneca Valley Virus (SVV) or a Coxsackievirus. In some embodiments, the SVV is an SVV-A selected from a wild type SVV-A (SEQ ID NO: 1), an S177A-SVVA mutant (SEQ ID NO: 2), an SVV-IR2 mutant (SEQ ID NO: 3), or an SVV-IR2-S177A (SEQ ID NO: 4). In some embodiments, the Coxsackievirus is selected from CVB3, CVA21, and CVA9. In some embodiments, the Coxsackievirus is a modified CVA21 virus comprising SEQ ID NO: 27.

In some embodiments, the recombinant RNA molecule further comprises a micro RNA (miRNA) target sequence (miR-TS) cassette inserted into the polynucleotide sequence encoding the oncolytic virus, wherein the miR-TS cassette comprises one or more miRNA target sequences, and wherein expression of one or more of the corresponding miRNAs in a cell inhibits replication of the encoded virus in the cell. In some embodiments, the one or more miRNAs are selected from miR-124, miR-1, miR-143, miR-128, miR-219, miR-219a, miR-122, miR-204, miR-217, miR-137, and miR-126. In some embodiments, the miR-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, miR-TS cassette comprises one or more copies of a miR-217 target sequence, one or more copies of a miR-137 target sequence, and one or more copies of a miR-126 target sequence. In some embodiments, the one or more miR-TS cassettes is incorporated into the 5′ untranslated region (UTR) or 3′ UTR of one or more essential viral genes. In some embodiments, the one or more miR-TS cassettes is incorporated into the 5′ untranslated region (UTR) or 3′ UTR of one or more non-essential genes. In some embodiments, the one or more miR-TS cassettes is incorporated 5′ or 3′ of one or more essential viral genes.

In some embodiments, the recombinant RNA molecule is capable of producing a replication-competent oncolytic virus when introduced into a cell by a non-viral delivery vehicle. In some embodiments, the cell is a mammalian cell. In some embodiments, the cell is a mammalian cell present in a mammalian subject.

In some embodiments, the replication-competent virus is selected from the group consisting of coxsackie virus, polio virus, Seneca valley virus, lassa virus, murine leukemia virus, influenza A virus, influenza B virus, Newcastle disease virus, measles virus, sindbis virus, and a maraba virus. In some embodiments, the replication-competent virus is selected from those listed in Table 1.

In some embodiments, the recombinant RNA molecule is inserted into a nucleic acid vector. In some embodiments, the nucleic acid vector is a replicon.

In some embodiments, the synthetic RNA viral genome further comprises a heterologous polynucleotide encoding an exogenous payload protein. In some embodiments, the exogenous payload protein is a fluorescent protein, an enzymatic protein, a cytokine, a chemokine, an antigen-binding molecule capable of binding to a cell surface receptor, or a ligand capable of binding to a cell surface receptor. In some embodiments, the cytokine is selected from IL-12, GM-CSF, IL-18, IL-2, and IL-36γ. In some embodiments, the ligand for a cell-surface receptor is Flt3 ligand or TNTSF14. In some embodiments, the chemokine is selected from CXCL10, CCL4, CCL21, and CCL5. In some embodiments, the antigen-binding molecule is capable of binding to and inhibiting an immune checkpoint receptor. In some embodiments, the immune checkpoint receptor is PD-1. In some embodiments, the antigen-binding molecule is capable of binding to a tumor antigen. In some embodiments, the antigen binding molecule is a bispecific T cell engager molecule (BiTE) or a bispecific light T cell engager molecule (LiTE). In some embodiments, the tumor antigen is DLL3 or EpCAM.

In some embodiments, the present disclosure provides a recombinant DNA molecule comprising from 5′ to 3′, a promoter sequence, a 5′ junctional cleavage sequence, a polynucleotide sequence encoding a recombinant RNA molecule described herein, and a 3′ junctional cleavage sequence. In some embodiments, the promoter sequence is a T7 promoter sequence.

In some embodiments, the 5′ junctional cleavage sequence is a ribozyme sequence and the 3′ junctional cleavage sequence is a ribozyme sequence. In some embodiments, the 5′ ribozyme sequence is a hammerhead ribozyme sequence and wherein the 3′ ribozyme sequence is a hepatitis delta virus ribozyme sequence. In some embodiments, the 5′ junctional cleavage sequence is a ribozyme sequence and the 3′ junctional cleavage sequence is a restriction enzyme recognition sequence. In some embodiments, the 5′ ribozyme sequence is a hammerhead ribozyme sequence, a Pistol ribozyme sequence, or a modified Pistol ribozyme sequence. In some embodiments, the 3′ restriction enzyme recognition sequence is a Type IIS restriction enzyme recognition sequence. In some embodiments, the Type IIS recognition sequence is a SapI recognition sequence. In some embodiments, the 5′ junctional cleavage sequence is an RNAseH primer binding sequence and the 3′ junctional cleavage sequence is a restriction enzyme recognition sequence.

In some embodiments, the present disclosure provides method of producing a recombinant RNA molecule described herein, comprising in vitro transcription of a DNA molecule described herein and purification of the resulting recombinant RNA molecule. In some embodiments, the recombinant RNA molecule comprises 5′ and 3′ ends that are native to the oncolytic virus encoded by the synthetic RNA viral genome.

In some embodiments, the present disclosure provides a composition comprising an effective amount of a recombinant RNA molecule described herein, and a carrier suitable for administration to a mammalian subject.

In some embodiments, the present disclosure provides a particle comprising a recombinant RNA molecule described herein. In some embodiments, the particle is biodegradable. In some embodiments, the particle is selected from the group consisting of a nanoparticle, an exosome, a liposome, and a lipoplex. In some embodiments, the exosome is a modified exosome derived from an intact exosome or an empty exosome. In some embodiments, the nanoparticle is a lipid nanoparticle (LNP) comprising cationic lipid, one or more helper lipids and a phospholipid-polymer conjugate.

In some embodiments, the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP). In some embodiments, the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol. In some embodiments, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

In some embodiments, the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

In some embodiments, the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

In some embodiments, the cationic lipid comprises COATSOME® SS-OC, wherein the one or more helper lipids comprise cholesterol (Chol) and DSPC, and wherein the phospholipid-polymer conjugate comprises DPG-PEG2000. In some embodiments, the ratio of SS-OC:DSPC:Chol:DPG-PEG2K (as a percentage of total lipid content) is A:B:C:D, wherein A=40%-60%, B=10%-25%, C=20%-30%, and D=0%-3% and wherein A+B+C+D=100%. In some embodiments, the ratio of SS-OC:DSPC:Chol:DPG-PEG2K (as a percentage of total lipid content) is A:B:C:D, wherein A=45%-50%, B=20%-25%, C=25%-30%, and D=0%-1% and wherein A+B+C+D=100%. In some embodiments, the ratio of SS-OC:DSPC:Chol:DPG-PEG2K (as a percentage of total lipid content) is about 49:22:28.5:0.5.

In some embodiments, the ratio of SS-OC:DSPC:Chol:DPG-PEG2K (as a percentage of total lipid content) is A:B:C:D, wherein A=40%-60%, B=10%-30%, C=20%-45%, and D=0%-3% and wherein A+B+C+D=100%. In some embodiments, the ratio of SS-OC:DSPC:Chol:DPG-PEG2K (as a percentage of total lipid content) is A:B:C:D, wherein A=40%-60%, B=10%-30%, C=25%-45%, and D=0%-3% and wherein A+B+C+D=100%. In some embodiments, the ratio of SS-OC:DSPC:Chol:DPG-PEG2K (as a percentage of total lipid content) is A:B:C:D, wherein A=45%-55%, B=10%-20%, C=30%-40%, and D=1%-2% and wherein A+B+C+D=100%. In some embodiments, the ratio of SS-OC:DSPC:Chol:DPG-PEG2K (as a percentage of total lipid content) is A:B:C:D, wherein A=45%-50%, B=10%-15%, C=35%-40%, and D=1%-2% and wherein A+B+C+D=100%. In some embodiments, the ratio of SS-OC:DSPC:Chol:DPG-PEG2K (as a percentage of total lipid content) is 49:11:38.5:1.5. In some embodiments, the TAP comprises a lipid formulation selected from Table 5.

In some embodiments, the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE). In some embodiments, the particle further comprises a phospholipid-polymer conjugate, wherein the phospholipid-polymer conjugate is 1,2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

In some embodiments, hyaluronan is conjugated to the surface of the LNP.

In some embodiments, the particle further comprises a second recombinant RNA molecule encoding a payload molecule. In some embodiments, the second recombinant RNA molecule is a replicon.

In some embodiments, the present disclosure provides a therapeutic composition comprising a plurality of lipid nanoparticles described herein. In some embodiments, the plurality of LNPs have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm to about 120 nm. In some embodiments, the plurality of LNPs have an average size of about 50 nm, 60 nm, 70 nm, 80 nm, 90 nm, 100 nm, 110 nm, or about 120 nm. In some embodiments, the plurality of LNPs have an average size of about 100 nm.

In some embodiments, the plurality of LNPs have an average zeta-potential of between about 20 mV to about −20 mV, about 10 mV to about −10 mV, about 5 mV to about −5 mV, or about 20 mV to about −40 mV. In some embodiments, the plurality of LNPs have an average zeta-potential of less than about −20 mV, less than about −30 mV, less than about 35 mV, or less than about −40 mV. In some embodiments, the plurality of LNPs have an average zeta-potential of between about −50 mV to about −20 mV, about −40 mV to about −20 mV, or about −30 mV to about −20 mV. In some embodiments, the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In some embodiments, delivery of the composition to a subject delivers the encapsulated recombinant RNA molecule to a target cell, and wherein the encapsulated recombinant RNA molecule produces an infectious virus capable of lysing the target cell. In some embodiments, the composition is formulated for intravenous or intratumoral delivery. In some embodiments, the target cell is a cancerous cell.

In some embodiments, the present disclosure provides an inorganic particle comprising a recombinant polynucleotide described herein. In some embodiments, the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP). In some embodiments, the inorganic particle further comprises a second recombinant RNA molecule encoding a payload molecule. In some embodiments, the second recombinant RNA molecule is a replicon.

In some embodiments, the present disclosure provides a composition comprising an inorganic particle described herein, wherein the average diameter of the particles is less than about 500 nm, is between about 50 nm and 500 nm, is between about 250 nm and about 500 nm, or is about 350 nm. In some embodiments, the present disclosure provides a composition comprising an inorganic particle described herein, wherein the average diameter of the particles is about 50 nm to about 120 nm. In some embodiments, the average diameter of the particles is about 50 nm, 60 nm, 70 nm, 80 nm, 90 nm, 100 nm, 110 nm, or about 120 nm. In some embodiments, the plurality of LNPs have an average size of about 100 nm.

In some embodiments, the present disclosure provides a method of killing a cancerous cell comprising exposing the cancerous cell to a particle described herein, a recombinant RNA molecule described herein, or compositions thereof, under conditions sufficient for the intracellular delivery of the particle to said cancerous cell, wherein the replication-competent virus produced by the encapsulated polynucleotide results in killing of the cancerous cell. In some embodiments, the replication-competent virus is not produced in non-cancerous cells. In some embodiments, the method is performed in vivo, in vitro, or ex vivo.

In some embodiments, the present disclosure provides a method of treating a cancer in a subject comprising administering to a subject suffering from the cancer an effective amount of a particle described herein, a recombinant RNA molecule described herein, or compositions thereof. In some embodiments, the particle or composition thereof is administered intravenously, intranasally, as an inhalant, or is injected directly into a tumor. In some embodiments, the particle or composition thereof is administered to the subject repeatedly. In some embodiments, the subject is a mouse, a rat, a rabbit, a cat, a dog, a horse, a non-human primate, or a human.

In some embodiments, the cancer is selected from lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, bladder cancer, melanoma, B-cell chronic lymphocytic leukemia, diffuse large B-cell lymphoma (DLBCL), sarcoma, and marginal zone lymphoma (MZL). In some embodiments, the lung cancer is small cell lung cancer or non-small cell lung cancer. In some embodiments, the liver cancer is hepatocellular carcinoma (HCC). In some embodiments, the prostate cancer is treatment-emergent neuroendocrine prostate cancer. In some embodiments, the bladder cancer, the pancreatic cancer, and the gastric cancer are a neuroendocrine subtype.

### BRIEF DESCRIPTION OF THE DRAWINGS

FIG. 1A-FIG. 1B shows production of RNA molecules comprising an SVV genome and viral lysis of SVV

FIG. 2 shows successful RNA delivery and functional virus production after treatment with SVV +ssRNA/LNPs

FIG. 3A-FIG. 3D shows variation in lipid nanoparticle composition alters particle size and/or percentage of RNA entrapment.

FIG. 4A-FIG. 4B shows efficacy of SVV +ssRNA/LNP in an H1299 tumor model. FIG. 4A shows tumor volume in H1299 tumor-bearing mice following intravenous administration of PBS or SVV +ssRNA/LNP (formulation ID: 70001-5.C). FIG. 4B shows body weight measurements of H1299 tumor-bearing mice intravenously treated with PBS or SVV +ssRNA/LNP (formulation ID: 70001-5.C).

FIG. 5A-FIG. 5B show recovery of infectious SVV from tumors after intravenous dosing of SVV +ssRNA/LNP.

FIG. 6A-FIG. 6D shows efficacy of SVV/LNP formulations 70009-1.C, 70009-2.C, and 70009-3.C in an H1299 tumor model. FIG. 6A shows tumor volume in H1299 tumor-bearing mice following intravenous administration of PBS, SVV-Neg/LNP (formulation ID: 70009-1.C), SVV/LNP (formulation ID: 70009-2.C), or SVV-S177A/LNP (formulation ID: 70009-3.C). FIG. 6B shows body weight measurements of H1299 tumor-bearing mice intravenously treated with PBS, SVV-Neg/LNP (formulation ID: 70009-1.C), SVV/LNP (formulation ID: 70009-2.C), or SVV-S177A/LNP (formulation ID: 70009-3.C). FIG. 6C-FIG. 6D shows SVV replication in tumor (FIG. 6C) or liver (FIG. 6D) tissue isolated from H1299 tumor-bearing mice treated with PBS, SVV-Neg/LNP (formulation ID: 70009-1.C), SVV/LNP (formulation ID: 70009-2.C), or SVV-S177A/LNP (formulation ID: 70009-3.C).

FIG. 7 shows tumor volume in H1299 tumor-bearing mice following intravenous administration of PBS or SVV-WT RNA lipid nanoparticles (formulation IDs: 70053-1-1.C, 70053-2-2.C, 70059-1-3.C, and 70059-2-4.C).

FIG. 8 shows tumor volume in H82 tumor-bearing mice following intravenous administration of PBS or SVV-WT (formulation ID: 70087-1.C) RNA lipid nanoparticles, or intratumoral administration of SVV-WT formulated with Lipofectamine.

FIG. 9A-FIG. 9D shows efficacy of SVV/LNP formulations 70077-3.C, 70077-4.C, 70077-8.C, 70077-10.C, and 70077-11.C in an H1299 tumor model. FIG. 9A shows tumor volume in H1299 tumor-bearing mice following intravenous administration of PBS or SVV-S177A RNA lipid nanoparticles (formulation IDs: 70077-3.C, 70077-4.C, 70077-8.C, 70077-10.C, and 70077-11.C). FIG. 9B shows body weight measurements of H1299 tumor-bearing mice intravenously treated with PBS or SVV-S177A RNA lipid nanoparticles (formulation IDs: 70077-3.C, 70077-4.C, 70077-8.C, 70077-10.C, and 70077-11.C). FIG. 9C shows serum aspartate aminotransferase (AST) and alanine aminotransferase (ALT) in H1299 tumor-bearing mice intravenously treated with PBS or SVV-S177A RNA lipid nanoparticles (formulation Ms: 70077-3.C, 70077-4.C, 70077-8.C. 70077-10.C, and 70077-11.C). FIG. 9D shows SVV replication in tumor tissue isolated from H1299 tumor-bearing mice intravenously treated with PBS or SVV-S177A RNA lipid nanoparticles (formulation IDs: 70077-3.C, 70077-4.C, 70077-8.C, 70077-10.C, and 70077-11.C).

FIG. 10 shows tumor volume in H1299 tumor-bearing mice following intravenous administration of PBS or SVV-S177A RNA lipid nanoparticles (formulation IDs: 70087-1.C, 70087-2.C, 70087-3.C, and 70087-4.C).

FIG. 11 shows SVV replication in tumor tissue isolated from H1299 tumor-bearing mice intravenously treated SVV-S177A RNA lipid nanoparticles (formulation IDs: 80010-1.C, 80010-2,C, 80010-3.C, 80010-4.C, and 80010-5.C).

FIG. 12 shows tumor volume in H1299 tumor-bearing mice following intravenous administration of PBS or SVV-S177A RNA lipid nanoparticles (formulation IDs: 80033-1.C, 80033-2.C, and 80033-3.C).

FIG. 13A FIG. 13C shows the efficacy of SVV/LNP formulation 80059-1.C and 80059-2.C in an H446 tumor model. FIG. 13A shows tumor volume in H446 tumor-bearing mice following intravenous administration of PBS, SVV-Neg LNPs (formulation ID: 80059-1.C), or SVV-S177A LNPs (formulation ID: 80059-2.C). FIG. 13B shows body weight measurements of H446 tumor-bearing mice intravenously treated with PBS, SVV-Neg LNPs (formulation ID: 80059-1.C), or SVV-S177A LNPs (formulation ID: 80059-2.C). FIG. 13C shows SVV replication in tumor tissue isolated from H446 tumor-bearing mice intravenously treated with PBS, SVV-Neg LNPs (formulation ID: 80059-1.C), or SVV-S177A LNPs (formulation ID: 80059-2.C).

FIG. 14 shows tumor volume in H1299 tumor-bearing mice following intravenous administration of PBS, SVV-WT LNPs (formulation ID: 80130-1.C), or SVV-IR2 LNPs (formulation ID: 80130-2.C).

FIG. 15 shows SVV replication in tumor tissue isolated from NIE-115 tumor-bearing mice intravenously treated with PBS, SVV-WT LNPs (formulation IL): 80130-1.C), or SVV-IR2 LNPs (formulation ID: 80130-2.C).

FIG. 16A-FIG. 16B shows inhibition of SVV-mediated H446 cell lysis upon treatment with anti-SVV polyclonal antibody.

FIG. 17 shows tumor volume in H1299 tumor-bearing mice following intravenous administration of PBS, SVV virus, or SVV-WT LNPs (formulation ID: 80139-1.C) and intraperitoneal administration of rabbit serum or anti-SVV polyclonal antibody.

FIG. 18 shows tumor volume in SK-MEL-28 tumor-bearing mice following intratumoral administration of PBS or CVA21-WT LNPs (formulation ID: 70032-6C), or intravenous administration of CVA21-WT LNPs.

FIG. 19 shows an overview of an in vitro transcriptional approach to generate an authentic 3′ terminus for picornaviruses using 3′ SapI restriction enzyme recognition sites.

FIG. 20 shows an RNaseH approach for generating an authentic 5′ terminus for picornaviruses using 5′ DNA primers and an RNaseH enzyme.

FIG. 21 illustrates a primer extension analysis of digested RNA with a 5′ RNaseH primer binding site and a 3′ SapI restriction site.

FIG. 22 shows a ribozyme approach for generating authentic 5′ termini for picornaviruses.

FIG. 23A-FIG. 23B show hammerhead ribozymes for generation of discrete 5′ termini. FIG. E-1 shows a structural model of a minimal hammerhead ribozyme (HHR) that anneals and cleaves at the 5′ terminus at the arrow. FIG. E-2 shows a structural model of a ribozyme with a stabilized stem I (STBL) for cleavage of 5′ terminus at the arrow.

FIG. 24A-FIG. 24B show pistol ribozymes for generation of discrete 5′ termini. FIG. F-1 shows a schematic of wild type Pistol ribozyme characteristics. FIG. F-2 shows Pistol ribozyme from P. polymyxa with a tetraloop added to fuse the P3 strands modeled by mFOLD. The dashed box is the area to retain the fold of the ribozyme in the context of the viral sequence. The “GUC” sequence shown in the dashed box was mutated to “UCA” to generate Pistol 1 and the “GUC” sequence was mutated to “TTA” to generate Pistol 2.

FIG. 25 demonstrates that the Pistol 1 ribozyme results in complete cleavage during the in vitro transcription process.

FIG. 26 illustrates primer extension analysis with all ribozymes during the in vitro transcription process.

FIG. 27 shows detection of minus-strand RNA, confirming that the Pistol 1 ribozyme results in faster kickoff of SVV replication from an RNA template compared to constructs using the 5′ Hammerhead ribozyme.

FIG. 28 shows the increased in vivo efficacy of the synthetic RNA SVV genomes generated with the 5′ Pistol 1 ribozyme and the 3′ SapI restriction site compared to constructs generated with the 5′ Hammerhead ribozyme. Tumor volume of H1299 tumor-bearing mice following intravenous administration of PBS, SVV-HHR LNPs (formulation ID: 80130-1.C), or SVV-PR LNPs (formulation ID: 80130-3.C).

FIG. 29A-FIG. 29B shows in vitro transcription of SVV RNA using modified ribonucleotides (FIG. 29A) and viral replication of SVV RNA genomes comprising the modified nucleotides (FIG. 29B).

FIG. 30 shows viral replication of SVV and SVV encoding various payload molecules from the SVV viral genomes (IC50 curves).

FIG. 31A-FIG. 31B shows efficacy of SVV-RNA genomes encoding IL-36γ in an H1299 tumor model. FIG. 31A shows tumor growth after treatment. FIG. 31B shows IL-36γ expression in tumor tissues.

FIG. 32A-FIG. 32B shows production of infectious CVA21 virus from RNA polynucleotides. FIG. 32A shows effects of 5′UTR sequences on the production of infectious CVA21 from RNA polynucleotides. FIG. 32B shows production of infectious CVA21 from RNA polynucleotides comprising the 5′ UTR of SEQ ID NO: 26.

FIG. 33 shows a schematic representation of LNP/SVV RNA composition and mode of action. LNP/SVV-RNA is systemically administered, SVV-RNA genomes are delivered to permissive tumor cells where they replicate and produce SVV-virions. SVV infection spreads to neighboring tumor cells eliciting oncolysis and antiviral immune responses.

FIG. 34 shows the in vitro transcription process for SVV-RNA and Neg-RNA. Autocatalytic cleavage of SVV-RNA by 5′ and 3′ ribozyme (Rib) generate SVV-RNA with discrete 5′ and 3′ ends required for replication. By contrast, Neg-RNA construct lacks ribozyme sequence and is not able of replication and virion production.

FIG. 35 shows the general schematic of using junctional cleavage sequences to remove non-viral RNA polynucleotides from the genome transcripts in order to maintain the native 5′ and 3′ discrete ends of the virus.

### DETAILED DESCRIPTION

There is a need in the art for viral therapies that are effective in the presence of neutralizing antibodies, able to be repeatedly systemically administered, and whose replication is limited to diseased cells, thus maximizing therapeutic efficacy while minimizing collateral damage to normal, non-cancerous cells. The present disclosure overcomes these obstacles and provides for replication-competent viral genomes that can be encapsulated in a non-immunogenic particle, such as a lipid nanoparticle, polymeric nanoparticle, or an exosome. In some embodiments, the particle further encapsulates a polynucleotide encoding a payload molecule. In some embodiments, the present disclosure provides replication-competent viral genomes and methods of use for the treatment and prevention of proliferative diseases and disorders (e.g., cancer). The present disclosure enables the systemic delivery of a safe, efficacious recombinant polynucleotide vector suitable to treat a broad array of proliferative disorders (e.g., cancers).

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences). In this application, the use of “or” means “and/or” unless stated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45. 50, 55, 60, 65, 70, 75, 80, 85. 90, 95, 99, or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

“Complementary” refers to the capacity for pairing, through base stacking and specific hydrogen bonding, between two sequences comprising naturally or non-naturally occurring (e.g., modified as described above) bases (nucleotides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

An “expression cassette” or “expression construct” refers to a polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject.

“Treating” as used herein refers to delivering an agent or composition to a subject to affect a physiologic outcome. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state; and (c) curing the disease.

The term “effective amount” refers to the minimum amount of an agent or composition required to result in a particular physiological effect (e.g., an amount required to increase, activate, and/or enhance a particular physiological effect). The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

“Effector function” refers to functions of an immune cell related to the generation, maintenance, and/or enhancement of an immune response against a target cell or target antigen.

The terms “microRNA,” “miRNA,” and “miR” are used interchangeably herein and refer to small non-coding endogenous RNAs of about 21-25 nucleotides in length that regulate gene expression by directing their target messenger RNAs (mRNA) for degradation or translational repression.

The term “composition” as used herein refers to a formulation of a recombinant RNA molecule or a particle-encapsulated recombinant RNA molecule described herein that is capable of being administered or delivered to a subject or cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

The term “replication-competent viral genome” refers to a viral genome encoding all of the viral genes necessary for viral replication and production of an infectious viral particle.

The term “oncolytic virus” refers to a virus that has been modified to, or naturally, preferentially infect cancer cells.

The term “vector” is used herein to refer to a nucleic acid molecule capable of transferring or transporting another nucleic acid molecule.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Synthetic RNA Viral Genomes

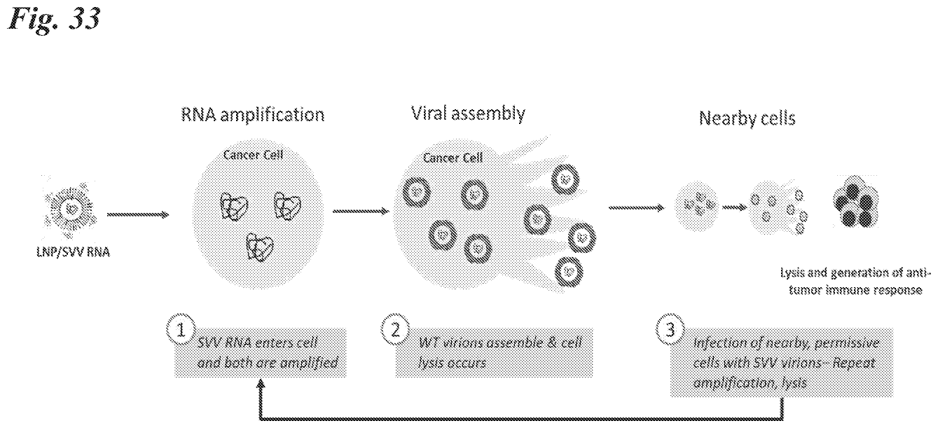

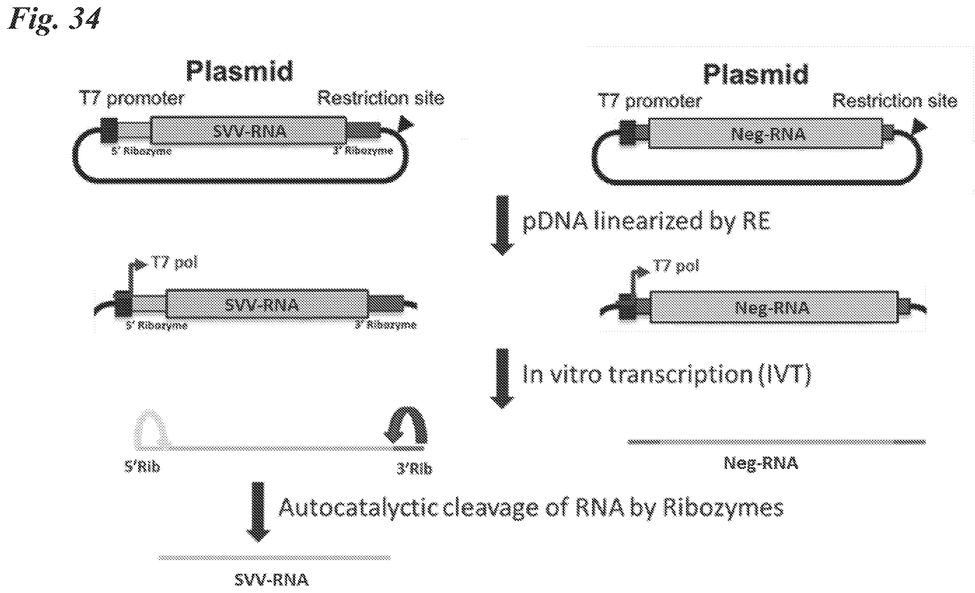

In some embodiments, the present disclosure provides a recombinant RNA molecule encoding an oncolytic virus (e.g., an RNA genome). Such recombinant RNA molecules are referred to herein as “synthetic viral genomes” or “synthetic RNA viral genomes”. In such embodiments, the synthetic RNA viral genome is capable of producing an infectious, lytic virus when introduced into a cell by a non-viral delivery vehicle and does not require additional exogenous genes or proteins to be present in the cell in order to replicate and produce an infectious virus. Rather, the endogenous translational mechanisms in the host cell mediate expression of the viral proteins from the synthetic RNA viral genome. The expressed viral proteins then mediate viral replication and assembly into an infectious viral particle (which may comprise a capsid protein, an envelope protein, and/or a membrane protein) comprising the RNA viral genome. As such, the RNA polynucleotides described herein (i.e., the synthetic RNA viral genomes), when introduced into a host cell, produce a virus that is capable of infecting another host cell. See schematic in FIG. 33

In some embodiments, the synthetic viral genome is provided as a recombinant ribonucleic acid (RNA) (i.e., a synthetic RNA viral genome). In some embodiments, the synthetic RNA viral genomes comprise one or more nucleic acid analogues. Examples of nucleic acid analogues include 2′-O-methyl-substituted RNA, 2′-O-methoxy-ethyl bases, 2′ Fluoro bases, locked nucleic acids (LNAs), unlocked nucleic acids (UNA), bridged nucleic acids (BNA), morpholinos, and peptide nucleic acids (PNA). In some embodiments, the synthetic RNA viral genome is a replicon, a RNA viral genome encoding a transgene, an mRNA molecule, or a circular RNA molecule (circRNA). In some embodiments, the synthetic RNA viral genome comprises a single stranded RNA (ssRNA) viral genome. In some embodiments, the single-stranded genome may be a positive sense or negative sense genome.

In some embodiments, the recombinant RNA molecule is a circular RNA molecule (circRNA). CircRNA molecules lack the free ends necessary for exonuclease mediated degradation, thus extending the half-life of the RNA molecule and enabling more stable protein production over time (See e.g., Wesselhoeft et al., Engineering circular RNA for potent and stable translation in eukaryotic cells. Nature Communications. (2018) 9:2629). In order to produce a functional RNA virus from a circRNA molecule, it is necessary to “break open” the circular construct once inside a cell so that the linear RNA genome with the appropriate 3′ and 5′ native ends can be produced. Therefore, in some embodiments, the recombinant RNA molecule encoding the oncolytic virus is provided as a circRNA molecule and further comprises one or more additional RNA sequences that facilitate the linearization of the circRNA molecule inside a cell. Examples of such additional RNA sequences include siRNA target sites, miRNA target sites, and guide RNA target sites. The corresponding siRNA, miRNA, or gRNA can be co-formulated with the circRNA molecule. Alternatively, the miRNA target site can be selected based on the expression of the cognate miRNA in a target cell, such that cleavage of the circRNA molecule and initial expression of the encoded oncolytic virus is limited to target cells expressing a particular miRNA.

The synthetic RNA viral genomes described herein encode an oncolytic virus. Examples of oncolytic viruses are known in the art including, but not limited to a picornavirus (e.g , a coxsackie virus), a polio virus, a measles virus, a vesicular stomatitis virus, an orthomyxovirus, and a maraba virus. In some embodiments, the oncolytic virus encoded by the synthetic RNA viral genome is a virus in the family Picornaviridae family such as a coxsackie virus, a polio virus (including a chimeric polio virus such as PVS-RIPO and other chimeric Picornaviruses), or a Seneca valley virus, or any virus of chimeric origin from any multitude of picornaviruses, a virus in the Arenaviridae family such a lassa virus, a virus in the Retroviridae family such as a murine leukemia virus, a virus in the family Orthomyxoviridae such as influenza A virus, a virus in the family Paramyxoviridae such as Newcastle disease virus or measles virus, a virus in the Reoviridae family such as mammalian orthoreovirus, a virus in the Togaviridae family such as sindbis virus, or a virus in the Rhabdoviridae family such as vesicular stomatitis virus (VSV) or a maraba virus.

### Positive-Sense, Single-Stranded RNA Viruses

In some embodiments, the synthetic RNA viral genomes described herein encode a single-stranded RNA (ssRNA) viral genome. In some embodiments, the ssRNA virus is a positive-sense, ssRNA (+sense ssRNA) virus. Exemplary+sense ssRNA viruses include members of the Picornaviridae family (e.g. coxsackie virus, poliovirus, and Seneca Valley virus (SVV), including SVV-A), the Coronaviridae family (e.g., Alphacoronaviruses such as HCoV-229E and HCoV-NL63, Betacoronoaviruses such as HCoV-HKU1, HCoV-OC3, and MERS-CoV), the Retroviridae family (e.g., Murine leukemia virus), and the Togaviridae family (e.g., Sindbis virus). Additional exemplary genera and species of positive-sense, ssRNA viruses are shown below in Table 1.

TABLE 1
Positive-sense ssRNA Viruses

| Family/Subfamily | Genus | Natural Host | Species |
|---|---|---|---|
| Picornaviridae | Cardiovirus | Human | |
| | Cosavirus | Human | |
| | Enterovirus | Human | Coxsackievirus |
| | | Human | Poliovirus |
| | Hepatovirus | Human | |
| | Kobuvirus | Human | |
| | Parechovirus | Human | |
| | Rosavirus | Human | |
| | Salivirus | Human | |
| | Pasivirus | Pigs | |
| | Senecavirus | Pigs | Seneca Valley |
| | | | Virus A |
| Caliciviridae | Sapovirus | Human | |
| | Norovirus | Human | |
| | Nebovirus | Bovine | |
| | Vesivirus | Felines/Swine | |
| Hepeviridae | Orthohepevirus | | |
| Astroviridae | Mamastrovirus | Human | |
| | Avastrovirus | Birds | |
| Flaviviridae | Hepacivirus | Human | |
| | Flavivirus | Arthropod | |
| | Pegivirus | | |
| | Pestivirus | Mammals | |
| Coronaviridae/ | Alphacoronavirus | | HCoV-229E |
| Coronavirinae | | | HCoV-NL63 |
| | Betacoronavirus | | HCoV-HKU1 |
| | | | HCoV-OC3 |
| | | | MERS-CoV |
| | Deltacoronavirus | | |
| | Gammacoronavirus | | |
| Coronaviridae/ | Bafinivirus | | |
| Torovirinae | Torovirus | | |
| Retroviridae | Gammaretrovirus | | Murine leukemia |
| | | | virus |
| Togaviridae | Alphavirus | | Sindbis virus |

In some embodiments, the recombinant RNA molecules described herein encode a Picornavirus selected from a coxsackie virus, poliovirus, and Seneca Valley virus (SVV). In some embodiments, the recombinant RNA molecules described herein encode a coxsackie virus. In some aspects of this embodiment, the recombinant RNA molecules a coxsackie virus and comprise the 5′ UTR sequence of SEQ ID NO: 26 (See e.g., Brown et al., Complete Genomic Sequencing Shows that Polioviruses and Members of Human Enterovirus Species C Are Closely Related in the Noncapsid Coding Region. Journal of Virology, (2003)77:16, p. 8973-8984. GenBank Accession No. AF546702). In such embodiments, the 5′ UTR sequence of SEQ ID NO: 26 unexpectedly increases the production of a functional coxsackie virus compared to other previously described 5′ UTR sequences (See e.g., Newcombe et al, Cellular receptor interactions of C-cluster human group A coxsackie viruses Journal of General Virology (2003), 84, 3041-3050. GenBank Accession No. AF465515). In some aspects of this embodiment, the recombinant RNA molecules encode a coxsackie virus and comprise the sequence of SEQ ID NO: 27.

In some embodiments, the synthetic RNA viral genomes described herein encode a coxsackie virus. In some embodiments, the coxsackie virus is selected from CVB3, CVA21, and CVA9. The nucleic acid sequences of exemplary coxsackie viruses are provided GenBank Reference No. M33854.1 (CVB3), GenBank Reference No. KT1.61266.1 (CVA21), and GenBank Reference No. D00627.1 (CVA9). In some embodiments, the synthetic RNA viral genomes described herein encode a modified CVA21 virus comprising SEQ ID NO: 27. In some embodiments, the synthetic RNA viral genomes described herein encode a Seneca Valley virus (SVV). In some embodiments, the SVV is selected from a wild-type SVV (such as SVV-A, SEQ ID NO: 1, GenBank Reference No. MF893200.1) or a mutant SVV (such as SVV-177A-SEQ ID NO: 2, SVV-IR2 SEQ ID NO: 3, or SVV-S177A-IR2-SEQ ID NO: 4). In some embodiments, the synthetic RNA viral genomes described herein encode a chimeric picornavirus (e.g., encode a virus comprising one portion, such as a capsid protein or an IRES, derived from a first picornavirus and another portion derived from a first picornavirus, and another portion, a non-structural gene such as a protease or polymerase derived from a second picornavirus). In some embodiments, the synthetic RNA viral genomes described herein encode a chimeric SVV. In some embodiments, the synthetic RNA viral genomes described herein encode a chimeric coxsackie virus.

In some embodiments, the synthetic RNA viral genome comprises a microRNA (miRNA) target sequence (miR-TS) cassette, wherein the miR-TS cassette comprises one or more miRNA target sequences, and wherein expression of one or more of the corresponding miRNAs in a cell inhibits replication of the encoded oncolytic virus in the cell. In some embodiments, the one or more miRNAs are selected from miR-124, miR-1, miR-143, miR-128, miR-219, miR-219a, miR-122, miR-204, miR-217, miR-137, and miR-126. In some embodiments, the miR.-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, the miR-TS cassette comprises one or more copies of a miR-217 target sequence, one or more copies of a miR-137 target sequence, and one or more copies of a miR-126 target sequence.

In some embodiments, the synthetic RNA viral genome comprises one or more miR-TS cassettes is incorporated into the 5′ untranslated region (UTR) or 3′ UTR of one or more essential viral genes. In some embodiments, the synthetic RNA viral genome comprises one or more miR-TS cassettes is incorporated into the 5′ untranslated region (UTR) or 3′ UTR of one or more non-essential genes. In some embodiments, the synthetic RNA viral genome comprises one or more miR-TS cassettes is incorporated 5′ or 3′ of one or more essential viral genes.

In some embodiments, the synthetic RNA viral genome comprises a heterologous polynucleotide encoding a payload molecule. In such embodiments, the synthetic RNA viral genome drives production of an infectious oncolytic virus as well as expression of the payload molecule. In such embodiments, the expression of the payload molecule can increase the therapeutic efficacy of the oncolytic virus. In some embodiments, the payload molecule is selected from IL-12 (e.g., an SVV genome encoding IL-12 such as SEQ ID NO: 8), GM-CSF (e.g., an SVV genome encoding GMCSF such as SEQ ID NO: 7), CXCL10 (e.g., an SVV genome encoding CXCL10 such as SEQ ID NO: 10), IL-36γ (e.g., an SVV genome encoding IL-36γ such as SEQ ID NO: 11), CCL21 (e.g., an SVV genome encoding CCL21), IL-18 (e.g., an SVV genome encoding IL-18), IL-2 (e.g., an SVV genome encoding IL-2), CCL4 (e.g., an SVV genome encoding CCL4), CCL5 (e.g., an SVV genome encoding CCL5), an anti-CD3-anti-FAP BiTE (e.g., an SVV genome encoding an anti-CD3-anti-FAP BiTE such as SEQ ID NO: 9), an antigen binding molecule that binds DLL3, or an antigen binding molecule that binds EpCAM. See Example 23 and 24, Further description of the types of payload molecules suitable for use in these embodiments is provided below.

Methods of Producing Recombinant RNA Viral Genomes

In some embodiments, the synthetic RNA viral genomes described herein are produced in vitro using one or more DNA vector templates comprising a polynucleotide encoding the synthetic RNA viral genomes. The term “vector” is used herein to refer to a nucleic acid molecule capable transferring, encoding, or transporting another nucleic acid molecule. The transferred nucleic acid is generally inserted into the vector nucleic acid molecule. A vector may include sequences that direct autonomous replication in a cell and/or may include sequences sufficient to allow integration into host cell DNA. In some embodiments, the recombinant RNA molecule encoding an oncolytic virus described herein is produced using one or more viral vectors.

In some embodiments, the synthetic RNA viral genomes described herein are produced by introducing a polynucleotide encoding the recombinant RNA molecule (e.g., by means of transfection, transduction, electroporation, and the like) into a suitable host cell in vitro. Suitable host cells include insect and mammalian cell lines. The host cells are cultured for an appropriate amount of time to allow expression of the polynucleotides and production of the synthetic RNA viral genomes. The synthetic RNA viral genomes are then isolated from the host cell and formulated for therapeutic use (e.g., encapsulated in a particle). A schematic of the in vitro synthesis of the RNA viral genomes with 3′ and 5′ ribozymes is shown in FIG. 34. The same schematic applies to the synthesis of RNA viral genomes using other combinations of junctional cleavage sequences.

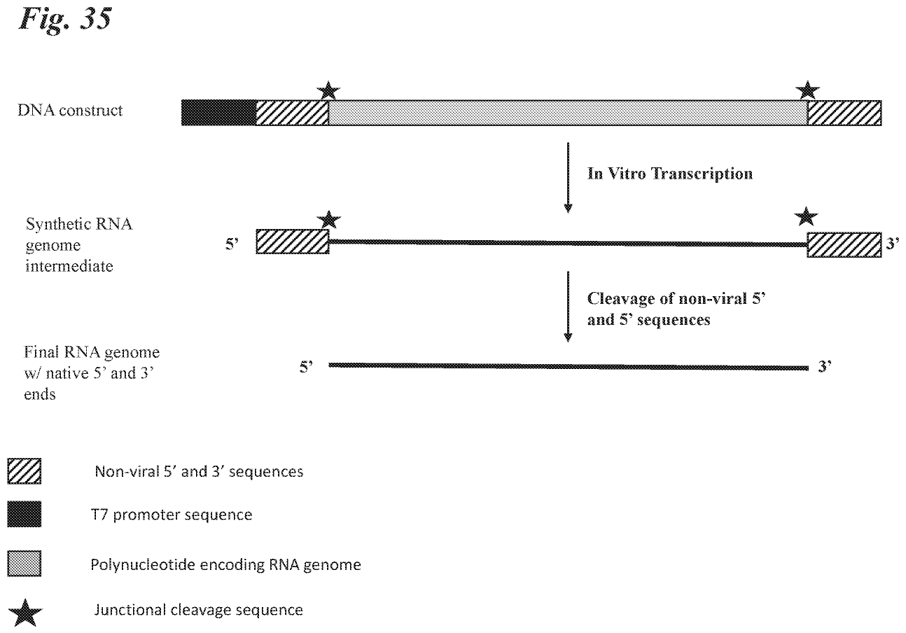

In some embodiments, the recombinant RNA molecules comprising the synthetic RNA viral genomes described herein require discrete 5′ and 3′ ends that are native to the virus. The RNA transcripts produced by T7 RNA polymerase in vitro or by mammalian RNA Pol II contain mammalian 5′ and 3′ UTRs do not contain the discrete, native ends required for production of an infectious RNA virus. For example, the T7 RNA polymerase requires a guanosine residue on the 5′ end of the template polynucleotide in order to initiate transcription. However, SVV begins with a uridine residue on its 5′ end. Thus, the T7 leader sequence, which is required for in vitro transcription of the SVV transcript must be removed to generate the native 5′ SVV terminus required for production of a functional infectious SVV. Therefore, in some embodiments, polynucleotides suitable for use in the production of the synthetic RNA viral genomes described herein require additional non-viral 5′ and 3′ sequences that enable generation of the discrete 5′ and 3′ ends native to the virus. Such sequences are referred to herein as junctional cleavage sequences (JCS). In some embodiments, the junctional cleavage sequences act to cleave the T7 RNA polymerase or Pol II-encoded RNA transcript at the junction of the viral RNA and the mammalian mRNA sequence such that the non-viral RNA polynucleotides are removed from the transcript in order to maintain the endogenous 5′ and 3′ discrete ends of the virus (See schematic shown in FIG. 35). In some embodiments, the junctional cleavage sequences act to generate the appropriate ends during the linearization of the DNA plasmid encoding the synthetic viral genome (e.g., the use of 3′ restriction enzyme recognition sequences to produce the appropriate 3′ end upon linearization of the plasmid template and prior to in vitro transcription of the synthetic RNA genome).

The nature of the junctional cleavage sequences and the removal of the non-viral RNA from the viral genome transcript can be accomplished by a variety of methods. For example, in some embodiments, the junctional cleavage sequences are targets for RNA interference (RNAi) molecules. “RNA interference molecule” as used herein refers to an RNA polynucleotide that mediates degradation of a target mRNA sequence through endogenous gene silencing pathways (e.g., Dicer and RNA-induced silencing complex (RISC)). Exemplary RNA interference agents include micro RNAs (miRNAs), artificial miRNA (amiRNAs), short hairpin RNAs (shRNAs), and small interfering RNAs (siRNAs). Further, any system for cleaving an RNA transcript at a specific site currently known the art or to be defined in the future can be used to generate the discrete ends native to the virus.

In some embodiments, the RNAi molecule is a miRNA. A miRNA refers to a naturally-occurring, small non-coding RNA molecule of about 18-25 nucleotides in length that is at least partially complementary to a target mRNA sequence. In animals, genes for miRNAs are transcribed to a primary miRNA (pri-miRNA), which is double stranded and forms a stem-loop structure. Pri-miRNAs are then cleaved in the nucleus by a microprocessor complex comprising the class 2 RNase III, Drosha, and the microprocessor subunit, DCGR8, to form a 70-100 nucleotide precursor miRNA (pre-miRNA). The pre-miRNA forms a hairpin structure and is transported to the cytoplasm where it is processed by the RNase III enzyme, Dicer, into a miRNA duplex of ˜ 18-25 nucleotides. Although either strand of the duplex may potentially act as a functional miRNA, typically one strand of the miRNA is degraded and only one strand is loaded onto the Argonaute (AGO) nuclease to produce the effector RNA-induced silencing complex (RISC) in which the miRNA and its mRNA target interact (Wahid et al., 1803:11, 2010, 1231-1243). In some embodiments, the 5′ and/or 3′ junctional cleavage sequences are miRNA target sequences.

In some embodiments, the RNAi molecule is an artificial miRNA (amiRNA) derived from a synthetic miRNA-embedded in a Pol II transcript. (See e.g., Liu et al., Nucleic Acids Res (2008) 36:9; 2811-2834; Zeng et al., Molecular Cell (2002), 9; 1327-1333; Fellman et al., Cell Reports (2013) 5; 1704-1713). In some embodiments, the 5′ and/or 3′ junctional cleavage sequences are amiRNA target sequences.

In some embodiments, the RNAi molecule is an siRNA molecule. siRNAs refer to double stranded RNA molecules typically about 21-23 nucleotides in length. The duplex siRNA molecule is processed in the cytoplasm by the associates with a multi protein complex called the RNA-induced silencing complex (RISC), during which the “passenger” sense strand is enzymatically cleaved from the duplex. The antisense “guide” strand contained in the activated RISC then guides the RISC to the corresponding mRNA by virtue of sequence complementarity and the AGO nuclease cuts the target mRNA, resulting in specific gene silencing. In some embodiments, the siRNA molecule is derived from an shRNA molecule. shRNAs are single stranded artificial RNA molecules ˜ 50-70 nucleotides in length that form stem-loop structures. Expression of shRNAs in cells is accomplished by introducing a DNA polynucleotide encoding the shRNA by plasmid or viral vector. The shRNA is then transcribed into a product that mimics the stem-loop structure of a pre-miRNA, and after nuclear export the hair-pin is processed by Dicer to form a duplex siRNA molecule which is then further processed by the RISC to mediate target-gene silencing. In some embodiments, the 5′ and/or 3′ junctional cleavage sequences are siRNA target sequences.

In some embodiments, the junctional cleavage sequences are guide RNA (gRNA) target sequences. In such embodiments, gRNAs can be designed and introduced with a Cas endonuclease with RNase activity (e.g., Cas13) to mediate cleavage of the viral genome transcript at the precise junctional site. In some embodiments, the 5′ and/or 3′ junctional cleavage sequences are gRNA target sequences.

In some embodiments, the junctional cleavage sequences are pri-miRNA-encoding sequences. Upon transcription of the polynucleotide encoding the viral genome (e.g., the recombinant RNA molecule), these sequences form the pri-miRNA stem-loop structure which is then cleaved in the nucleus by Drosha to cleave the transcript at the precise junctional site. In some embodiments, the 5′ and/or 3′ junctional cleavage sequences are pri-mRNA target sequences.

In some embodiments, the junctional cleavage sequences are primer binding sequences that facilitate cleavage by the endoribonuclease, RNAseH. In such embodiments, a primer that anneals to the 5′ and/or 3′ junctional cleavage sequence is added to the in vitro reaction along with an RNAseH enzyme. RNAseH specifically hydrolyzes the phosphodiester bonds of RNA which is hybridized to DNA, therefore enabling cleavage of the synthetic RNA genome intermediates at the precise junctional cleavage sequence to produce the required 5′ and 3′ native ends.

In some embodiments, the junctional cleavage sequences are restriction enzyme recognition sites and result in the generation of discrete ends of viral transcripts during linearization of the plasmid template runoff RNA synthesis with T7 RNA Polymerase. In some embodiments, the junctional cleavage sequences are Type IIS restriction enzyme recognition sites. Type IIS restriction enzymes comprise a specific group of enzymes which recognize asymmetric DNA sequences and cleave at a defined distance outside of their recognition sequence, usually within 1 to 20 nucleotides. Exemplary Type IIS restriction enzymes include AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBi, BsmnFI, BsmI, BspCNI, BspMI, BspQI, BsrDI, BsrI, BtgZI, BtsCI, BstI, CaspCI, EarI, EciI, Esp3I, FauI, FokI, HgaI, HphI, HpyAV, MbolI, MlyI, MmeI, MnlL, NmeAIII, PleI, SapI, and SfaNI. The recognition sequences for these Type IIS restriction enzymes are known in the art. See the New England Biolabs website located at neb.com/tools-and-resources/selection-charts/type-iis-restriction-enzymes. In some embodiments, the junctional cleavage sequence is a SapI restriction enzyme recognition site.

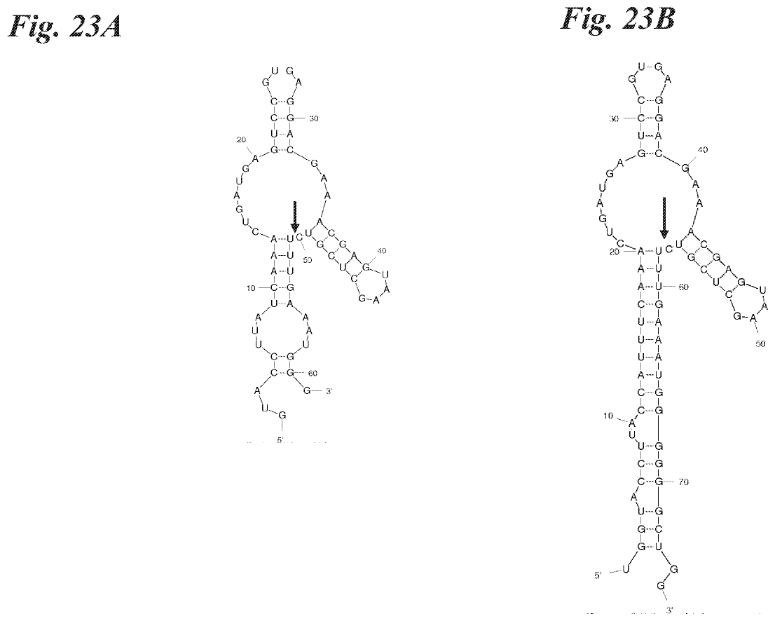

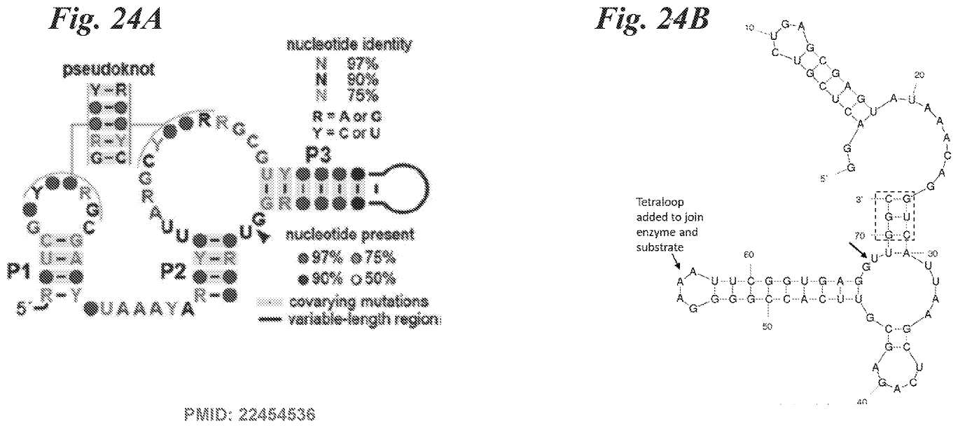

In some embodiments, the junctional cleavage sequences are ribozyme-encoding sequences and mediate self-cleavage of the synthetic RNA genome intermediates to produce the native discrete 5′ and 3′ ends of required for the final synthetic viral RNA genome and subsequent production of infectious RNA viruses. Exemplary ribozymes include the Hammerhead ribozyme (e.g., the Hammerhead ribozymes shown in FIG. 23), the Varkud satellite (VS) ribozyme, the hairpin ribozyme, the GIR1 branching ribozyme, the glmS ribozyme, the twister ribozyme, the twister sister ribozyme, the pistol ribozyme (e.g., Pistol 1 and Pistol 2 shown in FIG. 24), the hatchet ribozyme, and the Hepatitis delta virus ribozyme. In some embodiments, the 5′ and/or 3′ junctional cleavage sequences are ribozyme encoding sequences.

In some embodiments, the junctional cleavage sequences are sequences encoding ligand-inducible self-cleaving ribozymes, referred to as “aptazymes”. Aptazymes are ribozyme sequences that contain an integrated aptamer domain specific for a ligand. Ligand binding to the aptamer domain triggers activation of the enzymatic activity of the ribozyme, thereby resulting in cleavage of the RNA transcript. Exemplary aptazymes include theophylline-dependent aptazymes (e.g., hammerhead ribozyme linked to a theophylline-dependent aptamer, described in Auslander et al., Mol BioSyst. (2010) 6, 807-814), tetracycline-dependent aptazymes (e.g., hammerhead ribozyme linked to a Tet-dependent aptamer, described by Zhong et al., eLife 2016; 5:e18858 DOI: 10.7554/eLife.18858; Win and Smolke, PNAS (2007) 104; 14283-14288; Whittmann and Suess, Mol Biosyt (2011) 7; 2419-2427; Xiao et al., Chem & Biol (2008) 15; 125-1137; and Beilstein et al., ACS Syn Biol (2015) 4; 526-534), guanine-dependent aptazymes (e.g., hammerhead ribozyme linked to a guanine-dependent aptamer, described by Nomura et al., Chem Commun., (2012) 48(57); 7215-7217). In some embodiments, the 5′ and/or 3′ junctional cleavage sequences are aptazyme-encoding sequences.

In some embodiments, the junctional cleavage sequences are target sequences for an RNAi molecule (e.g., an siRNA molecule, an shRNA molecule, an miRNA molecule, or an amiRNA molecule), a gRNA molecule, or an RNAseH primer. In such embodiments, the junctional cleavage sequence is at least partially complementary to the sequence of the RNAi molecule, gRNA molecule, or primer molecule. Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), by manual alignment and visual inspection (see, e.g., Brent et al., (2003) Current Protocols in Molecular Biology), by use of algorithms known in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In some embodiments, the 5′ junctional cleavage sequence and 3′ junctional cleavage sequence are from the same group (e.g., are both RNAi target sequences, both ribozyme-encoding sequences, etc.). For example, in some embodiments, the junctional cleavage sequences are RNAi target sequences (e.g., siRNA, shRNA, amiRNA, or miRNA target sequences) and are incorporated into the 5′ and 3′ ends of the polynucleotide encoding the viral genome (e.g., the recombinant RNA molecule). In such embodiments, the 5′ and 3′ RNAi target sequence may be the same (i.e., targets for the same siRNA, amiRNA, or miRNA) or different (i.e, the 5′ sequence is a target for one siRNA, shmiRNA, or miRNA and the 3′ sequence is a target for another siRNA, amiRNA, or miRNA). In some embodiments, the junctional cleavage sequences are guide RNA target sequences and are incorporated into the 5′ and 3′ ends of the polynucleotide encoding the viral genome (e.g., the recombinant RNA molecule). In such embodiments, the 5′ and 3′ gRNA target sequences may be the same (i.e., targets for the same gRNA) or different (i.e., the 5′ sequence is a target for one gRNA and the 3′ sequence is a target for another gRNA). In some embodiments, the junctional cleavage sequences are pri-mRNA-encoding sequences and are incorporated into the 5′ and 3′ ends of the polynucleotide encoding the viral genome (e.g., the recombinant RNA molecule). In some embodiments, the junctional cleavage sequences are ribozyme-encoding sequences and are incorporated immediately 5′ and 3′ of the polynucleotide sequence encoding the viral genome (e.g., the recombinant RNA molecule).

In some embodiments, the 5′ junctional cleavage sequence and 3′ junctional cleavage sequence are from the same group but are different variants or types. For example, in some embodiments, the 5′ and 3′ junctional cleavage sequences may be target sequences for an RNAi molecule, wherein the 5′ junctional cleavage sequence is an siRNA target sequence and the 3′ junctional cleavage sequence is a miRNA target sequence (or vise versa). In some embodiments, the 5′ and 3′ junctional cleavage sequences may be ribozyme-encoding sequences, wherein the 5′ junctional cleavage sequence is a hammerhead ribozyme-encoding sequence and the 3′ junctional cleavage sequence is a hepatitis delta virus ribozyme-encoding sequence.

In some embodiments, the 5′ junctional cleavage sequence and 3′ junctional cleavage sequence are different types. For example, in some embodiments, the 5′ junctional cleavage sequence is an RNAi target sequence (e.g., an siRNA, an amiRNA, or a miRNA target sequence) and the 3′ junctional cleavage sequence is a ribozyme sequence, an aptazyme sequence, a pri-miRNA sequence, or a gRNA target sequence. In some embodiments, the 5′ junctional cleavage sequence is a ribozyme sequence and the 3′ junctional cleavage sequence is an RNAi target sequence (e.g., an siRNA, an amiRNA, or a miRNA target sequence), an aptazyme sequence, a pri-miRNA-encoding sequence, or a gRNA target sequence. In some embodiments, the 5′ junctional cleavage sequence is an aptazyme sequence and the 3′ junctional cleavage sequence is an RNAi target sequence (e.g., an siRNA, an amiRNA, or a miRNA target sequence), a ribozyme sequence, a pri-miRNA sequence, or a gRNA target sequence. In some embodiments, the 5′ junctional cleavage sequence is a pri-miRNA sequence and the 3′ junctional cleavage sequence is an RNAi target sequence (e.g., an siRNA, an amiRNA, or a miRNA target sequence), a ribozyme sequence, an aptazyme sequence, or a gRNA target sequence. In some embodiments, the 5′ junctional cleavage sequence is a gRNA target sequence and the 3′ junctional cleavage sequence is an RNAi target sequence (e.g., an siRNA, an amiRNA, or a miRNA target sequence), a ribozyme sequence, a pri-miRNA sequence, or an aptazyme sequence.

Exemplary arrangements of the junctional cleavage sequences relative to the polynucleotide encoding the synthetic viral genome are shown below in Tables A and B.

TABLE A
Symmetrical Junctional Cleavage Sequence (JSC) Arrangements

| 5′ | JCS | | JCS | 3′ |
|---|---|---|---|---|
| | siRNA TS | synthetic genome | siRNA TS | |
| | miR TS | synthetic genome | miR TS | |
| | AmiR TS | synthetic genome | AmiR TS | |
| | gRNA TS | synthetic genuine | gRNA TS | |
| | pri-miR | synthetic genome | pri-miR | |
| | ribozyme | synthetic genome | ribozyme | |
| | aptazyme | synthetic genome | aptazyme | |
| | RNAseH primer TS | synthetic genome | RNAseH primer TS | |

TABLE B
Asymmetrical JCS Arrangements

| 5′ | JCS | | JCS | 3′ |
|---|---|---|---|---|
| | siRNA TS | synthetic genome | miR TS | |
| | siRNA TS | synthetic genome | AmiR TS | |
| | siRNA TS | synthetic genome | gRNA TS | |
| | siRNA TS | synthetic genome | pri-miR | |
| | siRNA TS | synthetic genome | ribozyme | |
| | siRNA TS | synthetic genome | aptazyme | |
| | siRNA TS | synthetic genome | RNAseH primer TS | |
| | siRNA TS | synthetic genome | Restr Enz RS | |
| | miR TS | synthetic genome | siRNA TS | |
| | miR TS | synthetic genome | AmiR TS | |
| | miR TS | synthetic genome | gRNA TS | |
| | miR TS | synthetic genome | pri-miR | |
| | miR TS | synthetic genotne | ribozyme | |
| | miR TS | synthetic genome | aptazyme | |
| | miR TS | synthetic genome | RNAseH primer TS | |
| | miR TS | synthetic genome | Restr Enz RS | |
| | AmiR TS | synthetic genome | siRNA TS | |
| | AmiR TS | synthetic genome | miR TS | |
| | AmiR TS | synthetic genome | gRNA TS | |
| | AmiR TS | synthetic genome | pri-miR | |
| | AmiR TS | synthetic genome | ribozyme | |
| | AmiR TS | synthetic genome | aptazyme | |
| | AmiR TS | synthetic genome | RNAseH primer TS | |
| | AmiR TS | synthetic genome | Restr Enz RS | |
| | gRNA TS | synthetic genome | siRNA TS | |
| | gRNA TS | synthetic genome | miR TS | |
| | gRNA TS | synthetic genome | AmiR TS | |
| | gRNA TS | synthetic genome | pri-miR | |
| | gRNA TS | synthetic genome | ribozyme | |
| | gRNA TS | synthetic genome | aptazyme | |
| | gRNA TS | synthetic genome | RNAseH primer TS | |
| | gRNA TS | synthetic genome | Restr Enz RS | |
| | pri-miR | synthetic genome | siRNA TS | |
| | pri-miR | synthetic genome | miR TS | |
| | pri-miR | synthetic genotne | AmiR TS | |
| | pri-miR | synthetic genome | gRNA TS | |
| | pri-miR | synthetic genome | ribozyme | |
| | pri-miR | synthetic genome | aptazyme | |
| | pri-miR | synthetic genome | RNAseH primer TS | |
| | pri-miR | synthetic genome | Restr Enz RS | |
| | ribozyme | synthetic genome | siRNA TS | |
| | ribozyme | synthetic genome | miR TS | |
| | ribozyme | synthetic genome | AmiR TS | |
| | ribozyme | synthetic genome | gRNA TS | |
| | ribozyme | synthetic genome | pri-miR | |
| | ribozyme | synthetic genome | aptazyme | |
| | ribozyme | synthetic genome | RNAseH primer TS | |
| | ribozyme | synthetic genome | Restr Enz RS | |
| | aptazyme | synthetic genome | siRNA TS | |
| | aptazyme | synthetic genome | miR TS | |
| | aptazyme | synthetic genotne | AmiR TS | |
| | aptazyme | synthetic genome | gRNA TS | |
| | aptazyme | synthetic genome | pri-miR | |
| | aptazyme | synthetic genome | ribozyme | |
| | aptazyme | synthetic genome | RNAseH primer TS | |
| | aptazyme | synthetic genome | Restr Enz RS | |
| | RNAseH primer TS | synthetic genome | siRNA TS | |
| | RNAseH primer TS | synthetic genome | miR TS | |
| | RNAseH primer TS | synthetic genome | AmiR TS | |
| | RNAseH primer TS | synthetic genome | gRNA TS | |
| | RNAseH primer TS | synthetic genome | pri-miR | |
| | RNAseH primer TS | synthetic genome | ribozyme | |
| | RNAseH primer TS | synthetic genome | aptazyme | |
| | RNAseH primer TS | synthetic genome | Restr Enz RS | |

In some embodiments, the synthetic RNA viral genomes described herein are produced in vitro by In vitro RNA transcription (See schematic in FIG. 35). The synthetic RNA viral genomes are then purified and formulated for therapeutic use (e.g., encapsulated into a lipid nanoparticle). In some embodiments, the DNA polynucleotide comprises, from 5′ to 3′: (i) a promoter sequence (e.g., a T7 polymerase promoter); (ii) a 5′ ribozyme sequence; (iii) a polynucleotide encoding the synthetic RNA viral genome; and (iv) a 3′ ribozyme sequence. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding the synthetic RNA viral genome; and (iv) a 3′ hepatitis delta virus ribozyme sequence.

In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding a wild type SVV-A genome; and (iv) a 3′ hepatitis delta virus ribozyme sequence. In some embodiments, the DNA polynucleotide comprises a nucleic acid sequence that is at least 95%, 96%, 97%, 98%, or 99% identical to SEQ ID NO: 12. In some embodiments, the DNA polynucleotide comprises or consists of SEQ ID NO: 12. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding an SVVA-S177A genome; and (iv) a 3′ hepatitis delta virus ribozyme sequence. In some embodiments, the DNA polynucleotide comprises a nucleic acid sequence that is at least 95%, 96%, 97%, 98%, or 99% identical to SEQ ID NO: 13. In some embodiments, the DNA polynucleotide comprises or consists of SEQ ID NO: 13.

In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding an SVVA-IR2 genome; and (iv) a 3′ hepatitis delta virus ribozyme sequence. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding an SVVA-IR2-S77A genome; and (iv) a 3′ hepatitis delta virus ribozyme sequence.

In some embodiments, the DNA polynucleotide comprises, from 5′ to 3′: (i) a promoter sequence (e.g., a T7 polymerase promoter); (ii) a 5′ ribozyme sequence; (iii) a polynucleotide encoding the synthetic RNA viral genome; and (iv) a 3′ restriction enzyme recognition site. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding the synthetic RNA viral genome; and (iv) a 3′ SapI restriction enzyme recognition site.

In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding a wild-type SVVA genome, and (iv) a 3′ SapI restriction enzyme recognition site. In some embodiments, the DNA polynucleotide comprises a nucleic acid sequence that is at least 95%, 96%, 97%, 98%, or 99% identical to SEQ ID NO: 18. In some embodiments, the DNA polynucleotide comprises or consists of SEQ m NO: 18. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding the SVVA-S177A genome; and (iv) a 3′ SapI restriction enzyme recognition site. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 23); (iii) a polynucleotide encoding the SVVA-IR2 genome; and (iv) a 3′ SapI restriction enzyme recognition site. In some embodiments, the DNA polynucleotide comprises, from 5′ to 3′: (i) a promoter sequence (e.g., a T7 polymerase promoter); (ii) a 5′ Hammerhead ribozyme sequence (e.g., a wild type MIR or a modified IIIIR such as that provided in FIG. 23); (iii) a polynucleotide encoding a SVVA-S177A-IR2 genome; and (iv) a 3′ SapI restriction enzyme recognition site.

In some embodiments, the DNA polynucleotide comprises, from 5′ to 3′: (i) a promoter sequence (e.g., a T7 polymerase promoter); (ii) a 5′ Pistol ribozyme sequence (e.g., a Pistol 1 or a Pistol 2 ribozyme sequence shown in FIG. 24); (iii) a polynucleotide encoding the synthetic RNA viral genome; and (iv) a 3′ SapI restriction enzyme recognition site. In some embodiments, the DNA polynucleotide comprises, from 5′ to 3′: (i) a promoter sequence (e.g., a T7 polymerase promoter); (ii) a 5′ Pistol 1 ribozyme sequence; (iii) a polynucleotide encoding a wild type SVV genome; and (iv) a 3′ SapI restriction enzyme recognition site. In some embodiments, the DNA polynucleotide comprises a nucleic acid sequence that is at least 95%, 96%, 97%, 98%, or 99% identical to SEQ ID NO: 14. In some embodiments, the DNA polynucleotide comprises or consists of SEQ ID NO: 14. In some embodiments, the DNA polynucleotide comprises, from 5′ to 3′: (i) a promoter sequence (e.g., a T7 polymerase promoter); (ii) a 5′ Pistol 2 ribozyme sequence; (iii) a polynucleotide encoding a wild type SVV genome; and (iv) a 3′ SapI restriction enzyme recognition site. In some embodiments, the DNA polynucleotide comprises a nucleic acid sequence that is at least 95%, 96%, 97%, 98%, or 99% identical to SEQ ID NO: 15. In some embodiments, the DNA polynucleotide comprises or consists of SEQ m NO: 15.

In some embodiments, the DNA polynucleotide comprises, from 5′ to 3′: (i) a promoter sequence (e.g., a T7 polymerase promoter); (ii) a 5′ Pistol 1 ribozyme sequence; a polynucleotide encoding the SVV-S177A genome; and (iv) a 3′ SapI restriction enzyme recognition site. In some embodiments, the DNA polynucleotide comprises, from 5′ to 3′: (i) a promoter sequence (e.g., a T7 polymerase promoter); (ii) a 5′ Pistol 1 ribozyme sequence; (iii) a polynucleotide encoding the SVV-IR2 genome; and (iv) a 3′ SapI restriction enzyme recognition site. In some embodiments, the DNA polynucleotide comprises a nucleic acid sequence that is at least 95%, 96%, 97%, 98%, or 99% identical to SEQ ID NO: 16. In some embodiments, the DNA polynucleotide comprises or consists of SEQ ID NO: 16. In some embodiments, the DNA polynucleotide comprises, from 5′ to 3′: (i) a promoter sequence (e.g., a T7 polymerase promoter); (ii) a 5′ Pistol 1 ribozyme sequence; (iii) a polynucleotide encoding the SVV-IR2-S177A genome; and (iv) a 3′ SapI restriction enzyme recognition site. In some embodiments, the DNA polynucleotide comprises a nucleic acid sequence that is at least 95%, 96%, 97%, 98%, or 99% identical to SEQ ID NO: 17. In some embodiments, the DNA polynucleotide comprises or consists of SEQ ID NO: 17.

In some embodiments, the DNA polynucleotide comprises, from 5′ to 3′: (i) a promoter sequence (e.g., a T7 polymerase promoter); (ii) a 5′ RN AseH primer binding site; (iii) a polynucleotide encoding the synthetic RNA viral genome; and (iv) a 3′ restriction enzyme recognition site. In some embodiments, the DNA vector comprises a polynucleotide comprising, from 5′ to 3′: (i) a promoter sequence (e.g., a T7 polymerase promoter); (ii) a 5′ RNAseH primer binding site; (iii) a polynucleotide encoding the synthetic RNA viral genome; and (iv) a 3′ SapI restriction enzyme recognition site.

Particles Comprising Synthetic RNA Genomes

In some embodiments, the synthetic RNA genomes described herein are encapsulated in “particles.” As used herein, a particle refers to a non-tissue derived composition of matter such as liposomes, lipoplexes, nanoparticles, nanocapsules, microparticles, microspheres, lipid particles, exosomes, vesicles, and the like. In certain embodiments, the particles are non-proteinaceous and non-immunogenic. In such embodiments, encapsulation of the synthetic RNA genomes described herein allows for delivery of a viral genome without the induction of a systemic, anti-viral immune response and mitigates the effects of neutralizing anti-viral antibodies. Further, encapsulation of the synthetic RNA genomes described herein shields the genomes from degradation and facilitates the introduction into target host cells. In some embodiments, the present disclosure provides a nanoparticle comprising a synthetic RNA genome described herein. In some embodiments, the nanoparticle is a lipid nanoparticle. In some embodiments, the nanoparticle further comprises a second RNA molecule encoding a payload molecule.

In some embodiments, the particle is biodegradable in a subject. In such embodiments, multiple doses of the particles can be administered to a subject without an accumulation of particles in the subject. Examples of suitable particles include polystyrene particles, poly(lactic-co-glycolic acid) PLGA particles, polypeptide-based cationic polymer particles, cyclodextrin particles, chitosan particles, lipid based particles, poly(β-amino ester) particles, low-molecular-weight polyethylenimine particles, polyphosphoester particles, disulfide cross-linked polymer particles, polyamidoamine particles, polyethylenimine (PEI) particles, and PLURIONICS stabilized polypropylene sulfide particles.

In some embodiments, the polynucleotides described herein are encapsulated in inorganic particles. In some embodiments, the inorganic particles are gold nanoparticles (GNP), gold nanorods (GNR), magnetic nanoparticles (MNP), magnetic nanotubes (MNT), carbon nanohorns (CNH), carbon fullerenes, carbon nanotubes (CNT), calcium phosphate nanoparticles (CPNP), mesoporous silica nanoparticles (MSN), silica nanotubes (SNT), or a starlike hollow silica nanoparticles (SHNP).

Preferably, the particles described herein are nanoscopic in size, in order to enhance solubility, avoid possible complications caused by aggregation in vivo and to facilitate pinocytosis. In some embodiments, the particle has an average diameter of about less than about 1000 nm. In some embodiments, the particle has an average diameter of less than about 500 nm. In some embodiments, the particle has an average diameter of between about 30 and about 100 nm, between about 50 and about 100 nm, or between about 75 and about 100 nm. In some embodiments, the particle has an average diameter of between about 30 and about 75 nm or between about 30 and about 50 nm. In some embodiments, the particle has an average diameter between about 100 and about 500 nm. In some embodiments, the particle has an average diameter between about 200 and 400 nm. In some embodiments, the particle has an average size of about 350 nm.

### Exosomes

In some embodiments, the synthetic RNA genomes described herein are encapsulated in exosomes. Exosomes are small membrane vesicles of endocytic origin that are released into the extracellular environment following fusion of multivesicular bodies with the plasma membrane of the parental cell (e.g., the cell from which the exosome is released, also referred to herein as a donor cell). The surface of an exosome comprises a lipid bilayer derived from the parental cell's cell membrane and can further comprise membrane proteins expressed on the parental cell surface. In some embodiments, exosomes may also contain cytosol from the parental cell. Exosomes are produced by many different cell types including epithelial cells, B and T lymphocytes, mast cells (MC), and dendritic cells (DC) and have been identified in blood plasma, urine, bronchoalveolar lavage fluid, intestinal epithelial cells, and tumor tissues. Because the composition of an exosome is dependent on the parental cell type from which they are derived, there are no “exosome-specific” proteins. However, many exosomes comprise proteins associated with the intracellular vesicles from which the exosome originated in the parental cells (e.g., proteins associated with and/or expressed by endosomes and lysosomes). For example, exosomes can be enriched in antigen presentation molecules such as major histocompatibility complex I and II (MHC-I and MHC-II), tetraspanins (e.g., CD63), several heat shock proteins, cytoskeletal components such as actins and tubulins, proteins involved in intracellular membrane fusion, cell-cell interactions (e.g. CD54), signal transduction proteins, and cytosolic enzymes.

Exosomes may mediate transfer of cellular proteins from one cell (e.g., a parental cells) to a target or recipient cell by fusion of the exosomal membrane with the plasma membrane of the target cell. As such, modifying the material that is encapsulated by the exosome provides a mechanism by which exogenous agents, such as the polynucleotides described herein, may be introduced to a target cell. Exosomes that have been modified to contain one or more exogenous agents (e.g., a polynucleotide described herein) are referred to herein as “modified exosomes”. In some embodiments, modified exosomes are produced by introduction of the exogenous agent (e.g., a polynucleotide described herein) are introduced into a parental cell. In such embodiments, an exogenous nucleic acid is introduced into the parental, exosome-producing cells such that the exogenous nucleic acid itself, or a transcript of the exogenous nucleic acid is incorporated into the modified exosomes produced from the parental cell. The exogenous nucleic acids can be introduced to the parental cell by means known in the art, for example transduction, transfection, transformation, and/or microinjection of the exogenous nucleic acids.

In some embodiments, modified exosomes are produced by directly introducing a synthetic RNA genome described herein into an exosome. In some embodiments, a synthetic RNA genome described herein is introduced into an intact exosome. “Intact exosomes” refer to exosomes comprising proteins and/or genetic material derived from the parental cell from which they are produced. Methods for obtaining intact exosomes are known in the art (See e.g., Alvarez-Erviti L. et al., Nat Biotechnol. 2011 April; 29(4):34-5; Ohno S, et al., Mol Ther 2013 January; 21(1):185-91; and EP Patent Publication No. 2010663),

In particular embodiments, synthetic RNA genomes are introduced into empty exosomes. “Empty exosomes” refer to exosomes that lack proteins and/or genetic material (e.g., DNA or RNA) derived from the parental cell. Methods to produce empty exosomes (e.g., lacking parental cell-derived genetic material) are known in the art including UV-exposure, mutation/deletion of endogenous proteins that mediate loading of nucleic acids into exosomes, as well as electroporation and chemical treatments to open pores in the exosomal membranes such that endogenous genetic material passes out of the exosome through the open pores. In some embodiments, empty exosomes are produced by opening the exosomes by treatment with an aqueous solution having a pH from about 9 to about 14 to obtain exosomal membranes, removing intravesicular components (e.g., intravesicular proteins and/or nucleic acids), and reassembling the exosomal membranes to form empty exosomes. In some embodiments, intravesicular components (e.g., intravesicular proteins and/or nucleic acids) are removed by ultracentrifugation or density gradient ultracentrifugation. In some embodiments, the membranes are reassembled by sonication, mechanical vibration, extrusion through porous membranes, electric current, or combinations of one or more of these techniques. In particular embodiments, the membranes are reassembled by sonication.

In some embodiments, loading of intact or empty exosomes with a synthetic RNA genome described herein to produce a modified exosome can be achieved using conventional molecular biology techniques such as in vitro transformation, transfection, and/or microinjection. In some embodiments, the exogenous agents (e.g., the polynucleotides described herein) are introduced directly into intact or empty exosomes by electroporation. In some embodiments, the exogenous agents (e.g., the polynucleotides described herein) are introduced directly into intact or empty exosomes by lipofection (e.g., transfection). Lipofection kits suitable for use in the production of exosome according to the present disclosure are known in the art and are commercially available (e.g., FuGENE® HD Transfection Reagent from Roche, and LIPOFECTAMINE™ 2000 from invitrogen). In some embodiments, the exogenous agents (e.g., the polynucleotides described herein) are introduced directly into intact or empty exosomes by transformation using heat shock. In such embodiments, exosomes isolated from parental cells are chilled in the presence of divalent cations such as Ca²⁺ (in CaCl₂) in order to permeabilize the exosomal membrane. The exosomes can then be incubated with the exogenous nucleic acids and briefly heat shocked (e.g., incubated at 42° C. for 30-120 seconds). In particular embodiments, loading of empty exosomes with exogenous agents (e.g., the polynucleotides described herein) can be achieved by mixing or co-incubation of the agents with the exosomal membranes after the removal of intravesicular components. The modified exosomes reassembled from the exosomal membranes will, therefore, incorporate the exogenous agents into the intravesicular space. Additional methods for producing exosome encapsulated nucleic acids are known in the art (See e.g., U.S. Pat. Nos. 9,889,210; 9,629,929; and 9,085,778; International PCT Publication Nos. WO 2017/161010 and WO 2018/039119).

Exosomes can be obtained from numerous different parental cells, including cell lines, bone-marrow derived cells, and cells derived from primary patient samples. Exosomes released from parental cells can be isolated from supernatants of parental cell cultures by means known in the art. For example, physical properties of exosomes can be employed to separate them from a medium or other source material, including separation on the basis of electrical charge (e.g., electrophoretic separation), size (e.g., filtration, molecular sieving, etc.), density (e.g., regular or gradient centrifugation) and Svedberg constant (e.g., sedimentation with or without external force, etc). Alternatively, or additionally, isolation can be based on one or more biological properties, and include methods that can employ surface markers (e.g., for precipitation, reversible binding to solid phase, FACS separation, specific ligand binding, non-specific ligand binding, etc.). Analysis of exosomal surface proteins can be determined by flow cytometry using fluorescently labeled antibodies for exosome-associated proteins such as CD63. Additional markers for characterizing exosomes are described in International PCT Publication No. WO 2017/161010. In yet further contemplated methods, the exosomes can also be fused using chemical and/or physical methods, including PEG-induced fusion and/or ultrasonic fusion.

In some embodiments, size exclusion chromatography can be utilized to isolate the exosomes. In some embodiments, the exosomes can be further isolated after chromatographic separation by centrifugation techniques (of one or more chromatography fractions), as is generally known in the art. In some embodiments, the isolation of exosomes can involve combinations of methods that include, but are not limited to, differential centrifugation as previously described (See Raposo, et al., J. Exp. Med. 183, 1161-1172 (1996)), ultracentrifugation, size-based membrane filtration, concentration, and/or rate zonal centrifugation.

In some embodiments, the exosomal membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans. Exemplary exosomal membrane compositions and methods for modifying the relative amount of one or more membrane component are described in International PCT Publication No. WO 2018/039119.

In some embodiments, the particles are exosomes and have a diameter between about 30 and about 100 nm, between about 30 and about 200 nm, or between about 30 and about 500 nm. In some embodiments, the particles are exosomes and have a diameter between about 10 nm and about 100 nm, between about 20 nm and about 100 nm, between about 30 nm and about 100 nm, between about 40 nm and about 100 nm, between about 50 nm and about 100 nm, between about 60 nm and about 100 nm, between about 70 nm and about 100 nm, between about 80 nm and about 100 nm, between about 90 nm and about 100 nm, between about 100 nm and about 200 nm, between about 100 nm and about 150 nm, between about 150 nm and about 200 nm, between about 100 nm and about 250 nm, between about 250 nm and about 500 nm, or between about 10 nm and about 1000 nm. In some embodiments, the particles are exosomes and have a diameter between about 20 nm and 300 nm, between about 40 nm and 200 nm, between about 20 nm and 250 nm, between about 30 nm and 150 nm, or between about 30 nm and 100 nm.

### Lipid Nanoparticles

In certain embodiments, the synthetic RNA viral genomes described herein are encapsulated in a lipid nanoparticle (LNP). In certain embodiments, the LNP comprises one or more lipids such as such as triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e,g. cetyl palmitate). In some embodiments, the LNP comprises one or more cationic lipids and one or more helper lipids. In some embodiments, the LNP comprises one or more cationic lipids, a cholesterol, and one or more neutral lipids

Cationic lipids refer to any of a number of lipid species that carry a net positive charge at a selected pH, such as physiological pH. Such lipids include, but are riot limited to 1,2-Dilinolenyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl-N,N,N-trimethylammonium chloride (DOTAP); 3-(N-(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C₁₈ alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA. Additional cationic lipids are described in U.S. Pat. Nos. 7,745,651; 5,208,036; 5,264,618; 5,279,833; 5,283,185; 5,753,613; and 5,785,992 incorporated herein by reference.

In some embodiments, the cationic lipids comprise a protonatable tertiary amine head group. Such lipids are referred to herein as ionizable lipids. Ionizable lipids refer to lipid species comprising an ionizable amine head group and typically comprising a pKa of less than about 7. Therefore, in environments with an acidic pH, the ionizable amine head group is protonated such that the ionizable lipid preferentially interacts with negatively charged molecules (e.g., nucleic acids such as the recombinant polynucleotides described herein) thus facilitating nanoparticle assembly and encapsulation. Therefore, in some embodiments, ionizable lipids can increase the loading of nucleic acids into lipid nanoparticles. In environments where the pH is greater than about 7 (e.g., physiologic pH of ≈ 7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle. In some embodiments, the LNP comprises an ionizable lipid, e.g., a 7.SS-cleavable and pH-responsive Lipid Like Material (such as the COATSOME® SS-Series). Additional examples of cationic or ionizable lipids suitable for the formulations and methods of the disclosure are described in, e.g., WO2018089540A1, WO2017049245A2, US20150174261, US2014308304, US 2015376115, WO201/199952, and WO2016/176330.

In some embodiments, the cationic lipid is an ionizable lipid selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyloxy)heptadecanedioate (L-319), or N-(2,3-dioleoyloxy)propyl)-N,N,N-trimethylammonium chloride (DOTAP). In some embodiments, the cationic ionizable lipid is DLin-MC3-DMA (MC3). In some embodiments, the cationic ionizable lipid is COATSOME® SS-LC. In some embodiments, the cationic ionizable lipid is COATSOME® SS-EC. In some embodiments, the cationic ionizable lipid is COATSOME® SS-OC. In some embodiments, the cationic ionizable lipid is COATSOME® SS-OP. In some embodiments, the cationic ionizable lipid is L-319. In some embodiments, the cationic ionizable lipid is DOTAP.

In some embodiments, the LNPs comprise one or more non-cationic helper lipids (neutral lipids). Exemplary neutral helper lipids include (1,2-dilauroyl-sn-glycero-3-phosphoethanolamine) (DLPE), 1,2-diphytanoyl-sn-glycero-3-phosphoethanolamine (DiPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-dioleoyl-sn-glycero-3-phosphocholine (DOPC), 1,2-distearoyl-sn-glycero-3-phosphoethanolamine (DSPE), ceramides, sphingomyelins, and cholesterol. In some embodiments, the one or more helper lipids are selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol. In some embodiments, the LNPs comprise DSPC. In some embodiments, the LNPs comprise DOPC. In some embodiments, the LNPs comprise DLPE. In some embodiments, the LNPs comprise DOPE.

The use and inclusion of polyethylene glycol (PEG)-modified phospholipids and derivatized lipids such as derivatized ceramides (PEG-CER), including N-octanoyl-sphingosine-1-[succinyl(methoxy polyethylene glycol)-2000] (C8 PEG-2000 ceramide) in the liposomal and pharmaceutical compositions described herein is also contemplated, preferably in combination with one or more of the compounds and lipids disclosed herein.

In some embodiments, the lipid nanoparticles may further comprise one or more of PEG-modified lipids that comprise a poly(ethylene)glycol chain of up to 5 kDa in length covalently attached to a lipid comprising one or more C6-C20 alkyls. In some embodiments, the LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)] (DSPE-PEG-amine). In some embodiments, the LNPs further comprise a PEG-modified lipid selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K) ; and 1,2-dimyristoyl-rac-glycero-3-methylpolyoxyethylene-2000 (DMG-PEG2K). In some embodiments, the LNPs further comprise DSPE-PEG5K. In some embodiments, the LNPs further comprise DPG-PEG2K. In some embodiments, the LNPs further comprise DSG-PEG2K. In some embodiments, the LNPs further comprise DMG-PEG2K. In some embodiments, the LNPs further comprise DSG-PEG5K. In some embodiments, the LNPs further comprise DMG-PEG5K. In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, about 1.0%, about 1.5%, about 2.0%, about 2.5%, or about 3.0% of the total lipid content in the lipid nanoparticle.

In some embodiments, the lipid is modified with a cleavable PEG lipid. Examples of PEG derivatives with cleavable bonds include those modified with peptide bonds (Kulkarni et al. (2014). Mmp-9 responsive PEG cleavable nanovesicles for efficient delivery of chemotherapeutics to pancreatic cancer. Mol Pharmaceutics 11:2390-9; Lin et al. (2015). Drug/dye-loaded, multifunctional peg-chitosan-iron oxide nanocomposites for methotraxate synergistically self-targeted cancer therapy and dual model imaging. ACS Appl Mater Interfaces 7:11908-20.), disulfide keys (Yan et al (2014). A method to accelerate the gelation of disulfide-crosslinked hydrogels. Chin J Polym Sci 33:118-27; Wu & Yan (2015). Copper nanopowder catalyzed cross-coupling of diaryl disulfides with aryl iodides in PEG-400. Synlett 26:537-42), vinyl ether bonds, hydrazone bonds (Kelly et al. (2016). Polymeric prodrug combination to exploit the therapeutic potential of antimicrobial peptides against cancer cells. Org Bioniol Chem 14:9278-86.), and ester bonds (Xu et al. (2008). Esterase-catalyzed dePEGylation of pH-sensitive vesicles modified with cleavable PEG-lipid derivatives. J Control Release 130:238-45). See also, Fang et al., (2017) Cleaveable PEGylation: a strategy for overcoming the “PEG dilemma” in efficient drug delivery. Drug Delivery 24:2, 22-32.

In some embodiments, the PEG lipid is an activated PEG lipid. Exemplary activated PEG lipids include PEG-NH2, PEG-MAL, PEG-NHS, and PEG-ALD. Such functionalized PEG lipids are useful in the conjugation of targeting moieties to lipid nanoparticles to direct the particles to a particular target cell or tissue (e.g., by the attachment of antigen-binding molecules, peptides, glycans, etc.).

In some embodiments, the LNP comprises a cationic lipid and one or more helper lipids, wherein the cationic lipid is DOTAP. In some embodiments, the LNP comprises a cationic lipid and one or more helper lipids, wherein the cationic lipid is DLin-MC3-DMA (MC3). In some embodiments, the LNP comprises a cationic lipid and one or more helper lipids, wherein the cationic lipid is COATSOME® SS-EC. In some embodiments, the LNP comprises a cationic lipid and one or more helper lipids, wherein the cationic lipid is COATSOME® SS-LC. In some embodiments, the LNP comprises a cationic lipid and one or more helper lipids, wherein the cationic lipid is COATSOME® SS-OC. In some embodiments, the LNP comprises a cationic lipid and one or more helper lipids, wherein the cationic lipid is COATSOME® SS-OP. In some embodiments, the LNP comprises a cationic lipid and one or more helper lipids, wherein the cationic lipid is L-319.

In some embodiments, the LNP comprises a cationic lipid and one or more helper lipids, wherein the one or more helper lipids comprises cholesterol. In some embodiments, the LNP comprises a cationic lipid and one or more helper lipids, wherein the one or more helper lipids comprises DLPE. In some embodiments, the LNP comprises a cationic lipid and one or more helper lipids, wherein the one or more helper lipids comprises DSPC. In some embodiments, the LNP comprises a cationic lipid and one or more helper lipids, wherein the one or more helper lipids comprises DOPE. In some embodiments, the LNP comprises a cationic lipid and one or more helper lipids, wherein the one or more helper lipids comprises DOPC.

In some embodiments, the LNP comprises a cationic lipid and at least two helper lipids, wherein the cationic lipid is DOTAP, and the at least two helper lipids comprise cholesterol and DLPE. In some embodiments, the LNP comprises a cationic lipid and at least two helper lipids, wherein the cationic lipid is MC3, and the at least two helper lipids comprise cholesterol and DSPC. In some embodiments, the at least two helper lipids comprise cholesterol and DOPE. In some embodiments, the at least two helper lipids comprise cholesterol and DSPC. In some embodiments, the LNP comprises a cationic lipid and at least three helper lipids, wherein the cationic lipid is DOTAP, and the at least three helper lipids comprise cholesterol, DLPE, and DSPE. In some embodiments, the LNP comprises a cationic lipid and at least three helper lipids, wherein the cationic lipid is MC3, and the at least three helper lipids comprise cholesterol, DSPC, and DMG. In some embodiments, the at least three helper lipids comprise cholesterol, DOPE, and DSPE. In some embodiments, the at least three helper lipids comprise cholesterol, DSPC, and DMG. In some embodiments, the LNP comprises DOTAP, cholesterol, and DLPE. In some embodiments, the LNP comprises MC3, cholesterol, and DSPC. In some embodiments, the LNP comprises DOTAP, cholesterol, and DOPE. In some embodiments, the LNP comprises DOTAP, cholesterol, DLPE, and DSPE. In some embodiments, the LNP comprises MC3, cholesterol, DSPC, and DMG. In some embodiments, the LNP comprises DOTAP, cholesterol, DLPE, and DSPE-PEG. In some embodiments, the LNP comprises MC3, cholesterol, DSPC, and DMG-PEG. In some embodiments, the LNP comprises DOTAP, cholesterol, DOPE, and DSPE. In some embodiments, the LNP comprises DOTAP, cholesterol, DOPE, and DSPE-PEG. In some embodiments, the LNP comprises SS-OC, DSPC, cholesterol, and DPG-PEG (e.g., DPG-PEG2K).

In some embodiments, the LNP comprises DOTAP, cholesterol (Chol), and DLPE, wherein the ratio of DOTAP:Chol:DLPE (as a percentage of total lipid content) is about 50:35:15. In some embodiments, the LNP comprises DOTAP, cholesterol (Chol), and DLPE, wherein the ratio of DOTAP:Chol:DOPE (as a percentage of total lipid content) is about 50:35:15. In some embodiments, the LNP comprises DOTAP, cholesterol (Chol), DLPE, DSPE-PEG, wherein the ratio of DOTP:Chol:DLPE (as a percentage of total lipid content) is about 50:35:15 and wherein the particle comprises about 0.2% DSPE-PEG. In some embodiments, the LNP comprises MC3, cholesterol (Chol), DSPC, and DMG-PEG, wherein the ratio of MC3:Chol:DSPC:DMG-PEG (as a percentage of total lipid content) is about 49:38.5:11:1.5.

In some embodiments, the LNP comprises SS-OC, DSPC, cholesterol (Chol), and DPG-PEG2K), wherein the ratio of SS-OC:DSPC:Chol:DPG-PEG2K (as a percentage of total lipid content) is about A:B:C:D, wherein A=40%-60%, B=10%-25%, C=20%-30%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-50%, B=20%-25%, C=25%-30%, and D=0%-1% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about 49:22:28.5:0.5.

In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=40%-60%, B=10%-30%, C=20%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=40%-60%, B=10%-30%, C=25%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-55%, B=10%-20%, C=30%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=45%-50%, B=10%-15%, C=35%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is 49:11:38.5:1.5.

In some embodiments, the LNP comprises SS-OC, DSPC, cholesterol (Chol), and DPG-PEG2K, wherein the ratio of SS-OC:DSPC:Chol:DPG-PEG2K, (as a percentage of total lipid content) is about A:B:C:D, wherein A=45%-65%, B=5%-20%, C=20%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=50%-60%, B=5%-15%, C =30%-45%, and D=0%-3% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=55%-60%, B=5%-15%, C=30%-40%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is about A:B:C:D, wherein A=55%-60%, B=5%-10%, C=30%-35%, and D=1%-2% and wherein A+B+C+D=100%. In some embodiments, the LNP comprises SS-OC, DSPC, cholesterol (Chol), and DPG-PEG2K, wherein the ratio of SS-OC:DSPC:Chol:DPG-PEG2K (as a percentage of total lipid content) is 58:7:33.5:1.5.

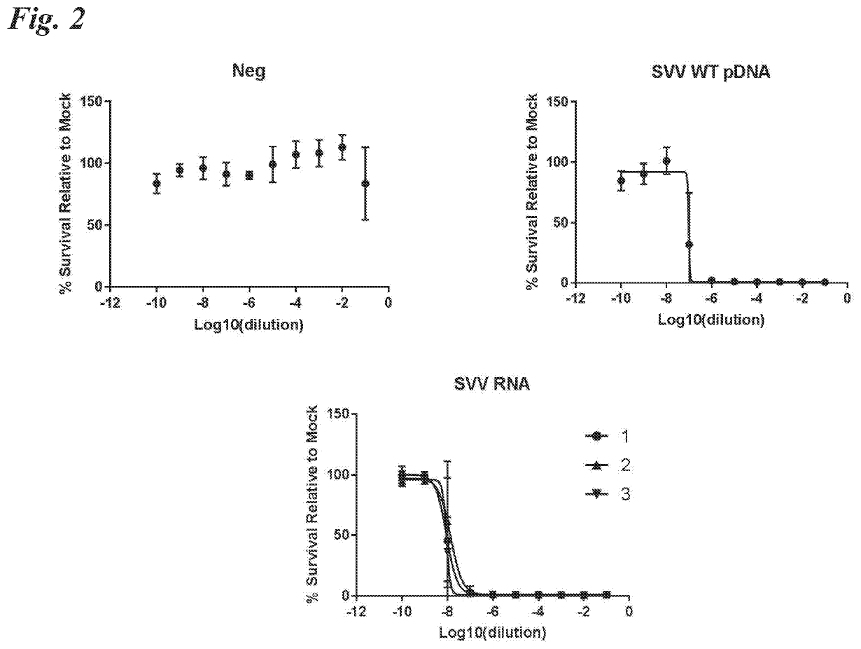

In some embodiments, the nanoparticle is coated with a glycosaminoglycan (GAG) in order to modulate or facilitate uptake of the nanoparticle by target cells (FIG. 2). The GAG may be heparin/heparin sulfate, chondroitin sulfate/dermatan sulfate, keratin sulfate, or hyaluronic acid (HA). In a particular embodiment, the surface of the nanoparticle is coated with HA and targets the particles for uptake by tumor cells. In some embodiments, the lipid nanoparticle is coated with an arginine-glycine-aspartate tri-peptide (RGD peptides) (See Ruoslahti, Advanced Materials, 24, 2012, 3747-3756; and Bellis et al., Biomaterials, 32(18), 2011, 4205-4210).

In some embodiments, the LNPs have an average size of about 50 nm to about 500 nm. For example, in some embodiments, the LNPs have an average size of about 50 nm to about 200 nm, about 100 nm to about 200 nm, about 150 nm to about 200 nm, about 50 nm to about 150 nm, about 100 nm to about 150 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm to about 120 nm. In some embodiments, the plurality of LNPs have an average size of about 50 nm, 60 nm, 70 nm, 80 nm, 90 nm, 100 nm, 110 nm, or about 120 nm. In some embodiments, the plurality of LNPs have an average size of about 100 nm.

In some embodiments, the LNPs have a neutral charge (e.g., an average zeta-potential of between about 0 mV and 1 mV). In some embodiments, the LNPs have an average zeta-potential of between about 40 mV and about −40 mV. In some embodiments, the LNPs have an average zeta-potential of between about 40 mV and about 0 mV. In some embodiments, the LNPs have an average zeta-potential of between about 35 mV and about 0 mV, about 30 mV and about 0 mV, about 25 mV to about 0 mV, about 20 mV to about 0 mV, about 15 mV to about 0 my, about 10 mV to about 0 mV, or about 5 mV to about 0 mV. In some embodiments, the LNPs have an average zeta-potential of between about 20 mV and about −40 mV. In some embodiments. the LNPs have an average zeta-potential of between about 20 mV and about −20 mV. In some embodiments, the LNPs have an average zeta-potential of between about 10 mV and about −20 mV. In some embodiments, the LNPs have an average zeta-potential of between about 10 mV and about −10 mV. In some embodiments, the LNPs have an average zeta-potential of about 10 mV, about 9 mV, about 8 mV, about 7 mV, about 6 mV, about 5 mV, about 4 mV, about 3 mV, about 2 mV, about 1 mV, about 0 mV, about −1 mV, about −2 mV, about −3 mV, about −4 mV, about −5 mV, about −6 mV, about −7 mV, about −8 mV, about −9 mV, about −9 mV or about −10 mV.

In some embodiments, the LNPs have an average zeta-potential of between about 0 mV and −20 mV. In some embodiments, the LNPs have an average zeta-potential of less than about −20 mV. For example in some embodiments, the LNPs have an average zeta-potential of less than about less than about −30 mV, less than about 35 mV, or less than about −40 mV. In some embodiments, the LNPs have an average zeta-potential of between about −50 mV to about −20 mV, about −40 mV to about −20 mV, or about −30 mV to about −20 mV. In some embodiments, the LNPs have an average zeta-potential of about 0 mV, about −1 mV, about −2 mV, about −3 mV, about −4 mV, about −5 mV, about −6 mV, about −7 mV, about −8 mV, about −9 mV, about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, about −30 mV, about −31 mV, about −32 mV, about −33 mV, about −34 mV, about −35 mV, about −36 mV, about −37 mV, about −38 mV, about −39 mV, or about −40 mV.

In some embodiments, the lipid nanoparticles comprise a recombinant nucleic acid molecule described herein and comprise a ratio of lipid (L) to nucleic acid (N) of about 3:1 (L:N). In some embodiments, the lipid nanoparticles comprise a recombinant nucleic acid molecule described herein and comprise an L:N ratio about 4:1, about 5:1, about 6:1, about 7:1, about 8:1, about 9:1, or about 10:1. In some embodiments, the lipid nanoparticles comprise a recombinant nucleic acid molecule described herein and comprise a ratio of lipid (L) to nucleic acid (N) of about 7:1. In some embodiments, the lipid nanoparticles comprise a recombinant nucleic acid molecule described herein and comprise an L:N ratio about 4.5:1, about 4.6:1, about 4.7:1, about 4.8:1, about 4.9:1, about 5:1, about 5.1:1, about 5.2:1, about 5.3:1, about 5.4:1, or about 5.5:1. In some embodiments, the lipid nanoparticles comprise a recombinant nucleic acid molecule described herein and comprise an L:N ratio about 6.5:1, 6.6:1, 6.7:1, 6.8:1, 6.9:1, 7:1, 7.1:1, 7.2:1, 7.3:1, 7.4: 1, and 7.5: 1.

In some embodiments, the LNP comprises a lipid formulation selected from one of the formulations listed in Table 5.

In some embodiments, the LNP comprises a synthetic RNA viral genome encoding an oncolytic virus, wherein the encoded oncolytic virus is capable of reducing the size of a tumor that is remote from the site of LNP administration to a subject. For example, as demonstrated in the examples provided herein, intravenous administration of the LNPs described herein results in viral replication in tumor tissue and reduction of tumor size. These data indicate that the LNPs of the present disclosure are capable of localizing to tumors or cancerous tissues that are remote from the site of LNP administration. Such effects enable the use of the LNP-encapsulated oncolytic viruses described herein in the treatment of tumors that are not easily accessible and therefore not suitable for intratumoral delivery of treatment.

### Payload Molecules

In some embodiments, the particles comprise a synthetic RNA viral genome and further comprise a recombinant RNA polynucleotide encoding a payload molecule. In some embodiments, the particles are lipid nanoparticles and comprise a synthetic RNA viral genome and further comprise a recombinant RNA polynucleotide encoding a payload molecule. In some embodiments, one or more miRNA target sequences are incorporated into the 3′ or 5′ UTR of the RNA polynucleotide encoding the payload molecule. In some embodiments, one or more miRNA target sequences are inserted into the polynucleotide encoding the payload molecule. In such embodiments, translation and subsequent expression of the payload does not occur, or is substantially reduced, in cells where the corresponding miRNA is expressed. In some embodiments, the recombinant RNA polynucleotide encoding a payload molecule is a replicon.

In some embodiments, the payload is a cytotoxic peptide. As used herein, a “cytotoxic peptide” refers to a protein capable of inducing cell death when expressed in a host cell and/or cell death of a neighboring cell when secreted by the host cell. In some embodiments, the cytotoxic peptide is a caspase, p53, diphtheria toxin (DT), Pseudomonas Exotoxin A (PEA), Type I ribozyme inactivating proteins (RIPs) (e.g., saporin and gelonin), Type II RIPs (e.g., ricin), Shiga-like toxin 1 (Slt1), photosensitive reactive oxygen species (e.g. killer-red). In certain embodiments, the cytotoxic peptide is encoded by a suicide gene resulting in cell death through apoptosis, such as a caspase gene.

In some embodiments, the payload is an immune modulatory peptide. As used herein, an “immune modulatory peptide” is a peptide capable of modulating (e.g., activating or inhibiting) a particular immune receptor and/or pathway. In some embodiments, the immune modulatory peptides can act on any mammalian cell including immune cells, tissue cells, and stromal cells. In a preferred embodiment, the immune modulatory peptide acts on an immune cell such as a T cell, an NK cell, an NKT T cell, a B cell, a dendritic cell, a macrophage, a basophil, a mast cell, or an eosinophil. Exemplary immune modulatory peptides include antigen-binding molecules such as antibodies or antigen binding fragments thereof, cytokines, chemokines, soluble receptors, cell-surface receptor ligands, bipartite peptides, and enzymes.

In some embodiments, the payload is a cytokine such as IL-1, IL-12, IL-15, IL-18, IL-36γ, TNFα, IFNα, IFNβ, IFNγ, or TNFSF14. In some embodiments, the payload is a chemokine such as CXCL10, CXCL9, CCL21, CCL4, or CCL5. In some embodiments, the payload is a ligand for a cell-surface receptor such as an NKG2D ligand, a neuropilin ligand, Flt3 ligand, a CD47 ligand SIRP1α). In some embodiments, the payload is a soluble receptor, such as a soluble cytokine receptor (e.g., IL-13R, TGFβR1, TGFβR2, IL-35R, IL-15R, IL-2R, IL-12R, and interferon receptors) or a soluble innate immune receptor (e.g., Toll-like receptors, complement receptors, etc.). In some embodiments, the payload is a dominant agonist mutant of a protein involved in intracellular RNA and/or DNA sensing (e.g. a dominant agonist mutant of STING, RIG-1, or MDA-5).

In some embodiments, the payload is an antigen-binding molecule such as an antibody or antigen-binding fragments thereof (e.g., a single chain variable fragment (scFv), an F(ab), etc.). In some embodiments, the antigen-binding molecule specifically binds to a cell surface receptor, such as an immune checkpoint receptor (e.g., PD-1, PD-L1, and CTLA4) or additional cell surface receptors involved in cell growth and activation (e.g., OX40, CD200R, CD47, CSF1R, 41BB, CD40, and NKG2D).

In some embodiments, the payload molecule is a scorpion polypeptide such as chlorotoxin, BmKn-2, neopladine 1, neopladine 2, and mauriporin. In some embodiments, the payload molecule is a snake polypeptide such as contortrostatin, apoxin-I, bothropstoxin-I, BJcuL, OHAP-1, rhodostomin, drCT-I, CTX-III, B1L, and ACTX-6. In some embodiments, the payload molecule is a spider polypeptide such as a latarcin and hyaluronidase. In some embodiments, the payload molecule is a bee polypeptide such as melittin and apamin. In some embodiments, the payload molecule is a frog polypeptide such as PsT-1, PdT-1, and PdT-2.

In some embodiments, the payload molecule is an enzyme. In some embodiments, the enzyme is capable of modulating the tumor microenvironment by way of altering the extracellular matrix. In such embodiments, the enzyme may include, but is not limited to, a matrix metalloprotease (e.g., MMP9), a collagenase, a hyaluronidase, a gelatinase, or an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P150. In some embodiments, the enzyme is capable of inducing or activating cell death pathways in the target cell (e.g., a caspase). In some embodiments, the enzyme is capable of degrading an extracellular metabolite or message (e.g. arginase or 15-Hydroxyprostaglandin Dehydrogenase).

In some embodiments, the payload molecule is a bipartite peptide. As used herein, a “bipartite peptide” refers to a multimeric protein comprised of a first domain capable of binding a cell surface antigen expressed on a non-cancerous effector cell and a second domain capable of binding a cell-surface antigen expressed by a target cell (e.g., a cancerous cell, a tumor cell, or an effector cell of a different type). In some embodiments, the individual polypeptide domains of a bipartite polypeptide may comprise an antibody or binding fragment thereof (e.g. a single chain variable fragment (scFv) or an F(ab)), a nanobody, a diabody, a flexibody, a DOCK-AND-LOCK™ antibody, or a monoclonal anti-idiotypic antibody (mAb2). In some embodiments, the structure of the bipartite polypeptides may be a dual-variable domain antibody (DVD-Ig™), Tandab®, a bi-specific T cell engager (BiTE™), a DuoBody®, or a dual affinity retargeting (DART) polypeptide. In some embodiments, the bipartite polypeptide is a BiTE and comprises a domain that specifically binds to an antigen shown in Table 3 and/or 4. Exemplary BiTEs are shown below in Table 2.

TABLE 2
Validated BiTEs used in preclinical and clinical studies

| Target | Name | Target Disease | Clinical Status | References |
|---|---|---|---|---|
| CD19 | Blinatumomab/MT- | NHL, ALL | Phase I/II/III | 1, 2, 3, 4, 5, 6 |
| | 103/MEDI-538 | | | |
| EpCAM | MT110 | Solid tumors | Phase I | 7, 8, 9, 10 |
| CEA | MT111/MEDI-565 | GI adenocarcinoma | Phase I | 11, 12 |
| PSMA | BAY2010112/AMG112 | Prostate | Phase I | 13 |
| CD33 | AMG330 | AML | Preclinical | 14, 15 |
| EGFR | C-BiTE and P-BiTE | Colorectal cancer | Preclinical | 16 |
| | antibodies | | | |
| Her2 | FynomAb, | Breast and gastric | Preclinical | 17, 18 |
| | COVA420, HER2- | carcinoma | | |
| | BsAb | | | |
| EphA2 | bscEphA2xCD3 | Multiple solid | Preclinical | 19 |
| | | tumors | | |
| MCSP | MCSP-BiTE | Melanoma | Preclinical | 20 |
| ADAM17 | A300E | Prostate cancer | Preclinical | 21 |
| PSCA | CD3-PSCA(MB1) | Prostate cancer | Preclinical | 22 |
| 17-A1 | CD3/17-1A-bispecific | Colorectal cancer | Preclinical | 23 |
| NKG2D | scFv-NKG2D, | Multiple solid and | Preclinical | 24, 25 |
| ligands | huNKG2D-OKT3 | liquid tumors | | |
| DLL3 | AMG757 | Small Cell Lung | Clinical | 26 |
| | | Cancer | | |

In some embodiments, the cell-surface antigen expressed on an effector cell is selected from Table 3 below. In some embodiments, the cell-surface antigen expressed on a tumor cell or effector cell is selected from Table 4 below. In some embodiments, the cell-surface antigen expressed on a tumor cell is a tumor antigen. In some embodiments, the tumor antigen is selected from CD19, EpCAM, CEA, PSMA, CD33, EGFR, Her2, EphA2, MCSP, ADAM17, PSCA, 17-A1, an NKGD2 ligand, CSF1R, FAP, GD2, DLL3, or neuropilin. In some embodiments, the tumor antigen is selected from those listed in Table 4.

TABLE 3
Exemplary effector cell target antigens

| T cell | | NKT cell | NK Cell | Other |
|---|---|---|---|---|
| CD3 | CD30 | CD3 | CD16 | CD48 |
| CD3γ | CD38 | CD3γ | CD94/NKG2 | LIGHT |
| | | | (e.g., NKG2D) | |
| CD3δ | CD40 | CD3δ | NKp30 | CD44 |
| CD3ε | CD57 | CD3ε | NKp44 | CD45 |
| CD3ξ | CD69 | CD3ξ | NKp46 | IL-1R2 |
| CD2 | CD70 | invariant TCR | KARs | IL-1Rα |
| CD4 | CD73 | | | IL- |
| | | | | 1Rα2 |
| CD5 | CD81 | | | IL- |
| | | | | 13Rα2 |
| CD6 | CD82 | | | IL- |
| | | | | 15Ra |
| CD7 | CD96 | | | CCR5 |
| CD8 | CD134 | | | CCR8 |
| CD16 | CD137 | | | |
| CD25 | CD152 | | | |
| CD27 | CD278 | | | |
| CD28 | | | | |

TABLE 4
Exemplary target cell antigens
Target Cell Antigens

| | | | |
|---|---|---|---|
| 8H9 | CRISP3 | Lewis-Y | Fas |
| GnT-V, β1, 6-N | DC-SIGN | LIV-1 (SLC39A6) | SOX2 |
| AFP | DHFR | Livin | STEAP1 |
| ART1 | EGP40 | LAMP1 | SLITRK6 |
| ART4 | EZH2 | MAGEA3 | NaPi2a |
| ABCG2 | EpCAM | MAGEA4 | SOX1 |
| B7-H3 | EphA2 | MAGEB6 | SOX11 |
| B7-H4 | EphA2/Eck | MAGEA1 | SPANXA1 |
| B7-H6 | EGFRvIII | MART-1 | SART-1 |
| BCMA | E-cadherin | MCSP | SSX4 |
| B-cyclin | EGP2 | MME | SSX5 |
| BMI1 | ETA | mesothelin (MSLN) | Survivin |
| CA-125 | ERBB3 | MAPK1 | SSX2 |
| cadherin | ERBB3/4 | MUC16 | TAG72 |
| CABYR | ERBB4 | MUC1 | TEM1 |
| CTAG2 | EPO | MRP-3 | TEM8 |
| CA6 | F3 | MyoD-1 | TSGA10 |
| CAIX | FAR | NCAM | TSSK6 |
| CEA | FBP | nectin 4 | thyroglobulin |
| CEACAM5 | FTHL17 | Nestin | transferrin receptor |
| CEACAM6 | fetal AchR | NEP | TACSTD2 (TROP2) |
| Cav-1 | FAP | NY-ESO-1 | TMEM97 |
| CD10 | FGFR3 | hHLA-A | TRP-2 |
| CD117 | FR-a | H60 | TULP2 |
| CD123 | Fra-1/Fosl 1 | OLIG2 | TROP2 |
| CD133 | GAGE1 | 5T4 | tyrosinase |
| CD138 | GD2 | p53 | TRP1 |
| CD15 | GD3 | P-Cadherin | UPAR |
| CD171 | Glil | PB | VEGF |
| CD19 | GP100 | P-glycoprotein | VEGF receptors |
| CD20 | GPA33 | PMCT (SLC13A5) | VEGRR2 |
| CD21 | | PRAME | BRAF |
| CD22 | Glypican-3 | PROXl | WT-1 |
| CD30 | HIV gp120 | PSA | XAGE2 |
| CD33 | HLA-A | PSCA | ZNF165 |
| CD37 | HLA-A2 | PSMA | αᵥβ₆ integrin |
| CD38 | HLA-AI | PSC1 | β-catenin |
| CD44v6 | HLA-B | PVRL4 | cathepsin B |
| CD44v7/8 | HLA-C | Ras | CSAG2 |
| CD74 | HMW-MAA | ROR1 | CTAG2 |
| Cd79b | Her2/Neu | SART2 | EGFR |
| CD124 (IL-4R) | Her3 | SART3 | EGP40 |
| CDH3 | u70/80 | oncofetal variants | EZH2 |
| | | of fibronectin | |
| Ki-67 | LICAM | tenascin | HIV sp120 |
| CSPG4 | ULBP1 | LICAM | kappa light chain |
| CALLA | ULBP2 | Rae-1α | LDHC |
| CSAG2 | ULBP3 | | TRP-1 |
| COX-2 | ULBP6 | Rae-1β | Fas-L |
| Lambda | MICA | Rae-lδ | DLL3 |
| LAYN | MICB | Rae-1γ | |
| LeuM-1 | Her3 | PDGF | |
| KDR | EGF | | |
| CD47 | SIRP1α | | |

Therapeutic compositions and Methods of Use

One aspect of the disclosure relates to therapeutic compositions comprising the recombinant RNA molecules described herein, or particles comprising a recombinant RNA molecule described herein, and methods for the treatment of cancer. Compositions described herein can be formulated in any manner suitable for a desired delivery route. Typically, formulations include all physiologically acceptable compositions including derivatives or prodrugs, solvates, stereoisomers, racemates, or tautomers thereof with any pharmaceutically acceptable carriers, diluents, and/or excipients.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine.

The present disclosure provides methods of killing a cancerous cell or a target cell comprising exposing the cell to an RNA polynucleotide or particle described herein, or composition thereof, under conditions sufficient for the intracellular delivery of the composition to the cancerous cell. As used herein, a “cancerous cell” or a “target cell” refers to a mammalian cell selected for treatment or administration with a polynucleotide or particle described herein, or composition thereof described herein. As used herein “killing a cancerous cell” refer specifically to the death of a cancerous cell by means of apoptosis or necrosis. Killing of a cancerous cell may be determined by methods known in the art including but not limited to, tumor size measurements, cell counts, and flow cytometry for the detection of cell death markers such as Annexin V and incorporation of propidium iodide.

The present disclosure further provides for a method of treating or preventing cancer in a subject in need thereof wherein an effective amount of the therapeutic compositions described herein is administered to the subject. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration. The encapsulated polynucleotide compositions described herein are particularly useful in the treatment of metastatic cancers, wherein systemic administration may be necessary to deliver the compositions to multiple organs and/or cell types. Therefore, in a particular embodiment, the compositions described herein are administered systemically.

An “effective amount” or an “effective dose,” used interchangeably herein, refers to an amount and or dose of the compositions described herein that results in an improvement or remediation of the symptoms of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. Improvements in subjects may include, but are not limited to, decreased tumor burden, decreased tumor cell proliferation, increased tumor cell death, activation of immune pathways, increased time to tumor progression, decreased cancer pain, increased survival, or improvements in the quality of life.

In some embodiments, administration of an effective dose may be achieved with administration a single dose of a composition described herein. As used herein, “dose” refers to the amount of a composition delivered at one time. In some embodiments, the dose of the recombinant RNA molecules is measured as the 50% Tissue culture Infective Dose (TCID₅₀). In some embodiments, the TCID₅₀ is at least about 10³-10⁹ TCID₅₀/mL, for example, at least about 10³ TCID₅₀/mL, about 10⁴ TCID₅₀/mL, about 10⁵ TCID₅₀/mL, about 10⁶ TCID₅₀/mL, about 10⁷ TCID₅₀/mL, about 10⁸ TCID₅₀/mL, or about 10⁹ TCID₅₀/mL. In some embodiments, a dose may be measured by the number of particles in a given volume (e.g., particles/mL). In some embodiments, a dose may be further refined by the genome copy number of the RNA polynucleotides described herein present in each particle (e.g., # of particles/mL, wherein each particle comprises at least one genome copy of the polynucleotide). In some embodiments, delivery of an effective dose may require administration of multiple doses of a composition described herein. As such, administration of an effective dose may require the administration of at least 2, 3, 4, 5, 6, 7, 8, 9, 10, 20, 30, 40, 50, or more doses of a composition described herein.

In embodiments wherein multiple doses of a composition described herein are administered, each dose need not be administered by the same actor and/or in the same geographical location. Further, the dosing may be administered according to a predetermined schedule. For example, the predetermined dosing schedule may comprise administering a dose of a composition described herein daily, every other day, weekly, bi-weekly, monthly, bi-monthly, annually, semi-annually, or the like. The predetermined dosing schedule may be adjusted as necessary for a given patient (e.g., the amount of the composition administered may be increased or decreased and/or the frequency of doses may be increased or decreased, and/or the total number of doses to be administered may be increased or decreased).

As used herein “prevention” or “prophylaxis” can mean complete prevention of the symptoms of a disease, a delay in onset of the symptoms of a disease, or a lessening in the severity of subsequently developed disease symptoms.

The term “subject” or “patient” as used herein, is taken to mean any mammalian subject to which a composition described herein is administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish, and reptiles.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is a neuroendocrine cancer. Furthermore, benign (i.e., noncancerous) hyperproliferative diseases, disorders and conditions, including benign prostatic hypertrophy (BPH), meningioma, schwannoma, neurofibromatosis, keloids, myoma and uterine fibroids and others may also be treated using the disclosure disclosed herein.

### Further Number Embodiments

Further numbered embodiments of the invention are provided as follows:

Embodiment 1: A lipid nanoparticle (LNP) comprising a synthetic RNA viral genome encoding an oncolytic virus.

Embodiment 2: The LNP of Embodiment 1, wherein the oncolytic virus is a single-stranded RNA (ssRNA) virus.

Embodiment 3: The LNP of Embodiment 1, wherein the oncolytic virus is a positive sense ((+)-sense) ssRNA virus.

Embodiment 4: The LNP of Embodiment 3, wherein the (+)-sense ssRNA virus is a Picornavirus.

Embodiment 5: The LNP of Embodiment 4, wherein the Picornavirus is a Seneca Valley Virus (SVV) or a Coxsackievirus.

Embodiment 6: The LNP of Embodiment 5, wherein the SVV is an SVV-A selected from a wild type SVV-A (SEQ ID NO: 1), an S177A-SVVA mutant (SEQ ID NO: 2), an SVV-IR2 mutant (SEQ ID NO: 3), and an SVV-IR2-S177A mutant (SEQ ID NO: 4).

Embodiment 7: The LNP of Embodiment 5, wherein the Coxsackievirus is selected from CVB3, CVA21, and CVA9.

Embodiment 8: The LNP of Embodiment 5, wherein the Coxsackievirus is a modified CVA21 virus comprising SEQ ID NO: 27.

Embodiment 9: The LNP of any one of Embodiments 1-8, wherein contacting the LNP with a cell results in production of viral particles by the cell, and wherein the viral particles are infectious and lytic.

Embodiment 10: The LNP of any one of Embodiments 1-9, wherein the synthetic RNA viral genome further comprises a heterologous polynucleotide encoding an exogenous payload protein

Embodiment 11: The LNP of any one of Embodiments 1-9, further comprising a recombinant RNA molecule encoding an exogenous payload protein.

Embodiment 12: The LNP of Embodiment 10 or 11, wherein the exogenous payload protein is a fluorescent protein, an enzymatic protein, a cytokine, a chemokine, an antigen-binding molecule capable of binding to a cell surface receptor, or a ligand for a cell-surface receptor.

Embodiment 13: The LNP of Embodiment 12, wherein the cytokine is selected from IL-12, GM-CSF, IL-18, IL-2, and IL-36γ.

Embodiment 14: The LNP of Embodiment 12, wherein the ligand for a cell-surface receptor is Flt3 ligand or TNFSF14.

Embodiment 15: The LNP of Embodiment 12, wherein the chemokine is selected from CXCL10, CCL4, CCL21, and CCL5.

Embodiment 16: The LNP of Embodiment 12, wherein the antigen-binding molecule is capable of binding to and inhibiting an immune checkpoint receptor.

Embodiment 17: The LNP of Embodiment 16, wherein the immune checkpoint receptor is PD-1.

Embodiment 18: The LNP of Embodiment 12, wherein the antigen-binding molecule is capable of binding to a tumor antigen.

Embodiment 19: The LNP of Embodiment 18, wherein the antigen binding molecule is a bispecific T cell engager molecule (BITE) or a bispecific light T cell engager molecule (LiTE).

Embodiment 20: The LNP of Embodiment 18 or 19, wherein the tumor antigen is DLL3 or EpCAM.

Embodiment 21: The LNP of any one of Embodiments 1-20, wherein the synthetic RNA viral genome and/or the recombinant RNA molecule comprises a micro RNA (miRNA) target sequence (miR-TS) cassette, wherein the miR-TS cassette comprises one or more miRNA target sequences.

Embodiment 22: The LNP of Embodiment 21, wherein the one or more miRNAs are selected from miR-124, miR-1, miR-143, miR-128, miR-219, miR-219a, miR-122, miR-204, miR-217, miR-137, and miR-126.

Embodiment 23: The LNP of Embodiment 22, wherein the miR-TS cassette comprises one or more copies of a miR-124 target sequence, one or more copies of a miR-1 target sequence, and one or more copies of a miR-143 target sequence.

Embodiment 24: The LNP of Embodiment 22, wherein the miR-TS cassette comprises one or more copies of a miR-128 target sequence, one or more copies of a miR-219a target sequence, and one or more copies of a miR-122 target sequence.

Embodiment 25: The LNP of Embodiment 22, wherein the miR-TS cassette comprises one or more copies of a miR-128 target sequence, one or more copies of a miR-204 target sequence, and one or more copies of a miR-219 target sequence.

Embodiment 26: The LNP of Embodiment 22, wherein the miR-TS cassette comprises one or more copies of a miR-217 target sequence, one or more copies of a miR-137 target sequence, and one or more copies of a miR-126 target sequence.

Embodiment 27: The LNP of any one of Embodiments 1-26, wherein the LNP comprises a cationic lipid, one or more helper lipids, and a phospholipid-polymer conjugate.

Embodiment 28: The LNP of Embodiment 27, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

Embodiment 29: The LNP of Embodiment 27 or 28, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol.

Embodiment 30: The LNP of Embodiment 27, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

Embodiment 31: The LNP of any one of Embodiments 27-30, wherein the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

Embodiment 32: The LNP of any one of Embodiments 27-31, wherein the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-alycero-3-methylpolyoxyethylene-2000 (DSG-PEG2K); 1,2-dimyristoy 1-rac-glycero-3-methylpolyoxyethylene-5000 (DMG-PEG5K); and 1,2-dimyristoyl-rac-alycero-3-methylpolyoxyethylene-2000 (DMG-PEG2K).

Embodiment 33: The LNP of Embodiment 27, wherein the cationic lipid comprises COATSOME® SS-OC, wherein the one or more helper lipids comprise cholesterol (Chol) and DSPC, and wherein the phospholipid-polymer conjugate comprises DPG-PEG2000.

Embodiment 34: The LNP of Embodiment 33, wherein the ratio of SS-OC:DSPC:Chol:DPG-PEG2K (as a percentage of total lipid content) is A:B:C:D, wherein A=40%-60%, B=10%-25%, C=20%-30%, and D=0%-3% and wherein A+B+C+D=100%.

Embodiment 35: The LNP of Embodiment 33, wherein the ratio of SS-OC:DSPC:Chol:DPG-PEG2K (as a percentage of total lipid content) is A:B:C:D, wherein A=45%-50%, B=20%-25%, C=25%-30%, and D=0%-1% and wherein A+B+C+D=100%.

Embodiment 36: The LNP of Embodiment 33, wherein the ratio of SS-OC:DSPC:Chol:DPG-PEG2K (as a percentage of total lipid content) is about 49:22:28.5:0.5.

Embodiment 37: The LNP of Embodiment 33, wherein the ratio of SS-OC:DSPC:Chol:DPG-PEG2K (as a percentage of total lipid content) is A:B:C:D, wherein A=40%-60%, B=10%-30%, C=20%-45%, and D=0%-3% and wherein A+B+C+D=100%.

Embodiment 37A: The LNP of Embodiment 33, wherein the ratio of SS-OC:DSPC:Chol:DPG-PEG2K (as a percentage of total lipid content) is A:B:C:D, wherein A=40%-60%, B=10%-30%, C=25%-45%, and D=0%-3% and wherein A+B+C+D=100%.

Embodiment 37B: The LNP of Embodiment 33, wherein the ratio of SS-OC:DSPC:Chol:DPG-PEG2K (as a percentage of total lipid content) is A:B:C:D, wherein A=45%-55%, B=10%-20%, C=30%-40%, and D=1%-2% and wherein A+B+C+D=100%.

Embodiment 38: The LNP of Embodiment 33, wherein the ratio of SS-OC:DSPC:Chol:DPG-PEG2K (as a percentage of total lipid content) is A:B:C:D, wherein A=45%-50%, B=10%-15%, C=35%-40%, and D=1%-2% and wherein A+B+C+D=100%.

Embodiment 39: The LNP of Embodiment 33, wherein the ratio of SS-OC:DSPC:Chol:DPG-PEG2K (as a percentage of total lipid content) is 49:11:38.5:1.5.

Embodiment 39A: The LNP of Embodiment 33, wherein the ratio of SS-OC:DSPC:Chol:DPG-PEG2K (as a percentage of total lipid content) is about A:B:C:D, wherein A=45%-65%, B=5%-20%, C=20%-45%, and D=0%-3% and wherein A+B+C+D=100%.

Embodiment 39B: The LNP of Embodiment 33, wherein the ratio of SS-OC:DSPC:Chol:DPG-PEG2K (as a percentage of total lipid content) is about A:B:C:D, wherein A=50%-60%, B=5%-15%, C=30%-45%, and D=0%-3% and wherein A+B+C+D=100%.

Embodiment 39C: The LNP of Embodiment 33, wherein the ratio of SS-OC:DSPC:Chol:DPG-PEG2K (as a percentage of total lipid content) is about A:B:C:D, wherein A=55%-60%, B=5%-15%, C=30%-40%, and D=1%-2% and wherein A+B+C+D=100%.

Embodiment 39D: The LNP of Embodiment 33, wherein the ratio of SS-OC:DSPC:Chol:DPG-PEG2K (as a percentage of total lipid content) is about A:B:C:D, wherein A=55%-60%, B=5%-10%, C=30%-35%, and D=1%-2% and wherein A+B+C+D=100%.

Embodiment 39E: The LNP of Embodiment 33, wherein the ratio of SS-OC:DSPC:Chol:DPG-PEG2K (as a percentage of total lipid content) is 58:7:33.5:1.5.

Embodiment 40: The LNP of any one of Embodiments 1-39E, wherein the LNP comprises a lipid formulation selected from Table 5.

Embodiment 41: The LNP of any one of Embodiments 1-40, wherein hyaluronan is conjugated to the surface of the LNP.

Embodiment 42: A therapeutic composition comprising a plurality of lipid nanoparticles according to any one of Embodiments 1-41.

Embodiment 43: The therapeutic composition of Embodiment 42, wherein the plurality of LNPs have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

Embodiment 43A: The therapeutic composition of Embodiment 42, wherein the plurality of LNPs have an average size of about 50 nm to about 120 nm.

Embodiment 43B: The therapeutic composition of Embodiment 42, wherein the plurality of LNPs have an average size of about 50 nm, 60 nm, 70 nm, 80 nm, 90 nm, 100 nm, 110 nm, or about 120 nm.

Embodiment 43C: The therapeutic composition of Embodiment 42, wherein the plurality of LNPs have an average size of about 100 nm.

Embodiment 44: The therapeutic composition of any one of Embodiments 42-43C, wherein the plurality of LNPs have an average zeta-potential of between about 40 mV to about −40 mV, 20 mV to about −20 mV, about 10 mV to about −10 mV, about 5 mV to about −5 mV, or about 20 mV to about −40 mV.

Embodiment 45: The therapeutic composition of any one of Embodiments 42-43C, wherein the plurality of LNPs have an average zeta-potential of less than about −20 mV, less than about −30 mV, less than about −35 mV, or less than about −40 mV.

Embodiment 46: The therapeutic composition of Embodiment 45, wherein the plurality of LNPs have an average zeta-potential of between about −50 mV to about −20 mV, about −40 mV to about −20 mV, or about −30 mV to about −20 mV.

Embodiment 47: The therapeutic composition of Embodiment 45 or 46, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

Embodiment 48: The therapeutic composition of any one of Embodiments 42-47, wherein administering the therapeutic composition to a subject delivers the recombinant RNA polynucleotide to a target cell of the subject, and wherein the recombinant RNA polynucleotide produces an infectious oncolytic virus capable of lysing the target cell of the subject.

Embodiment 49: The therapeutic composition of Embodiment 48, wherein the composition is formulated for intravenous or intratumoral delivery.

Embodiment 50: The therapeutic composition of Embodiment 48, wherein the target cell is a cancerous cell.

Embodiment 51: A method of inhibiting the growth of a cancerous tumor in a subject in need thereof comprising administering the therapeutic composition according to any one of Embodiments 42-50 to the subject in need thereof, wherein administration of the composition inhibits the growth of the tumor.

Embodiment 52: The method of Embodiment 51, wherein the administration is intratumoral or intravenous.

Embodiment 53: The method of Embodiment 51 or 52, wherein the cancer is a lung cancer, a liver cancer, a melanoma, a breast cancer, a pancreatic cancer, a prostate cancer, a neuroblastoma, a rhabdomyosarcoma, a medulloblastoma, or a bladder cancer.

Embodiment 53A: The method of any one of Embodiments 51-53, wherein the cancer is a neuroendocrine cancer.

Embodiment 54: A recombinant RNA molecule comprising a synthetic RNA viral genome encoding an oncolytic virus.

Embodiment 55: The recombinant RNA molecule of Embodiment 54, wherein the encoded oncolytic virus is a single-stranded RNA (ssRNA) virus

Embodiment 56: The recombinant RNA molecule of Embodiment 55, wherein the ssRNA virus is a positive sense ((+)-sense) or a negative-sense ((−)-sense) ssRNA virus.

Embodiment 57: The recombinant RNA molecule of Embodiment 56, wherein the (+)-sense ssRNA virus is a Picornavirus.

Embodiment 58: The recombinant RNA molecule of Embodiment 57, wherein the Picornavirus is a Seneca Valley Virus (SVV) or a Coxsackievirus.

Embodiment 59: The recombinant RNA molecule of Embodiment 58, wherein the SVV is an SVV-A selected from a wild type SVV-A (SEQ ID NO: 1), an S177A-SVVA mutant (SEQ ID NO: 2), an SVV-IR2 mutant (SEQ ID NO: 3), or an SVV-IR2-S177A (SEQ ID NO: 4).

Embodiment 60: The recombinant RNA molecule of Embodiment 58, wherein the Coxsackievirus is selected from CVB3, CVA21, and CNA9.

Embodiment 61: The recombinant RNA molecule of Embodiment 58, wherein the Coxsackievirus is a modified CVA21 virus comprising SEQ ID NO: 27.

Embodiment 62: The recombinant RNA molecule of any one of Embodiments 54-61, wherein the recombinant RNA molecule is capable of producing an infectious, lytic virus when introduced into a cell by a non-viral delivery vehicle.

Embodiment 63: The recombinant RNA molecule of any one of Embodiments 54-62, further comprising a micro RNA (miRNA) target sequence (miR-TS) cassette inserted into the polynucleotide sequence encoding the oncolytic virus, wherein the miR-TS cassette comprises one or more miRNA target sequences, and wherein expression of one or more of the corresponding miRNAs in a cell inhibits replication of the encoded virus in the cell.

Embodiment 64: The recombinant RNA molecule of Embodiment 63, wherein the one or more miRNAs are selected from miR-124, miR-1, miR-143, miR-128, miR-219, miR-219a, miR-122, miR-204, miR-217, miR-137, and miR-126.

Embodiment 65: The recombinant RNA molecule of Embodiment 64, wherein the miR-TS cassette comprises one or more copies of a miR-124 target sequence, one or more copies of a miR-1 target sequence, and one or more copies of a miR-143 target sequence.

Embodiment 66: The recombinant RNA molecule of Embodiment 64, wherein the miR-TS cassette comprises one or more copies of a miR-128 target sequence, one or more copies of a miR-219a target sequence, and one or more copies of a miR-122 target sequence.

Embodiment 67: The recombinant RNA molecule of Embodiment 64, wherein the miR-TS cassette comprises one or more copies of a miR-128 target sequence, one or more copies of a miR-204 target sequence, and one or more copies of a miR-219 target sequence.

Embodiment 68: The recombinant RNA molecule of Embodiment 64, wherein the miR-TS cassette comprises one or more copies of a miR-217 target sequence, one or more copies of a miR-137 target sequence, and one or more copies of a miR-126 target sequence.

Embodiment 69: The recombinant RNA molecule of any one of Embodiments 54-68, wherein the recombinant RNA molecule is capable of producing a replication-competent oncolytic virus when introduced into a cell by a non-viral delivery vehicle.

Embodiment 70: The recombinant RNA molecule of Embodiment 69, wherein the cell is a mammalian cell.

Embodiment 71: The recombinant RNA molecule of Embodiment 70, wherein the cell is a mammalian cell present in a mammalian subject.

Embodiment 72: The recombinant RNA molecule of any one of Embodiments 54-71, wherein the replication-competent virus is selected from the group consisting of coxsackie virus, polio virus, Seneca valley virus, lassa virus, murine leukemia virus, influenza A virus, influenza B virus, Newcastle disease virus, measles virus, sindbis virus, and a maraba virus.

Embodiment 72A: The recombinant RNA molecule of any one of Embodiments 54-71, wherein the replication-competent virus is selected from those listed in Table 1.

Embodiment 73: The recombinant RNA molecule of Embodiment 63, wherein the one or more miR-TS cassettes is incorporated into the 5′ untranslated region (UTR) or 3′ UTR of one or more essential viral genes.

Embodiment 74: The recombinant RNA molecule of Embodiment 63, wherein the one or more miR-TS cassettes is incorporated into the 5′ untranslated region (UTR) Of 3′ UTR of one or more non-essential genes.

Embodiment 74A: The recombinant RNA molecule of Embodiment 63, wherein the one or more miR-TS cassettes is incorporated 5′ or 3′ of one or more essential viral genes.

Embodiment 75: The recombinant RNA molecule of any of Embodiments 54-74A, wherein the recombinant RNA molecule is inserted into a nucleic acid vector.

Embodiment 76: The recombinant RNA molecule of Embodiment 75, wherein the nucleic acid vector is a replicon.

Embodiment 77: The recombinant RNA molecule of Embodiments 54-76, wherein the synthetic RNA viral genome further comprises a heterologous polynucleotide encoding an exogenous payload protein

Embodiment 78: The recombinant RNA molecule of Embodiment 77, wherein the exogenous payload protein is a fluorescent protein, an enzymatic protein, a cytokine, a chemokine, an antigen-binding molecule capable of binding to a cell surface receptor, or a ligand capable of binding to a cell surface receptor.

Embodiment 79: The recombinant RNA molecule of Embodiment 78, wherein the cytokine is selected from IL-12, GM-CSF, IL-2, and IL-36□.

Embodiment 80: The recombinant RNA molecule of Embodiment 78, wherein the ligand for a cell-surface receptor is Flt3 ligand or TNFSF14.

Embodiment 81: The recombinant RNA molecule of Embodiment 78, wherein the chemokine is selected from CXCL10, CCL4, CCL21, and CCL5.

Embodiment 82: The recombinant RNA molecule of Embodiment 78, wherein the antigen-binding molecule is capable of binding to and inhibiting an immune checkpoint receptor.

Embodiment 83: The recombinant RNA molecule of Embodiment 82, wherein the immune checkpoint receptor is PD-1.

Embodiment 84: The recombinant RNA molecule of Embodiment 78, wherein the antigen-binding molecule is capable of binding to a tumor antigen.

Embodiment 85: The recombinant RNA molecule of Embodiment 84, wherein the antigen binding molecule is a bispecific T cell engager molecule (BiTE) or a bispecific light T cell engager molecule (LiTE).

Embodiment 86: The recombinant RNA molecule of Embodiment 84 or 85, wherein the tumor antigen is DLL3 or EpCAM.

Embodiment 87: A recombinant DNA molecule comprising from 5′ to 3′, a promoter sequence, a 5′ junctional cleavage sequence, a polynucleotide sequence encoding the recombinant RNA molecule of any one of Embodiments 54-86, and a 3′ junctional cleavage sequence.

Embodiment 88: The recombinant DNA molecule of Embodiment 87, wherein the promoter sequence is a T7 promoter sequence.

Embodiment 89: The recombinant DNA molecule of Embodiment 87 or 88, wherein the 5′ junctional cleavage sequence is a ribozyme sequence and the 3′ junctional cleavage sequence is a ribozyme sequence.

Embodiment 90: The recombinant DNA molecule of Embodiment 89, wherein the 5′ ribozyme sequence is a hammerhead ribozyme sequence and wherein the 3′ ribozyme sequence is a hepatitis delta virus ribozyme sequence.

Embodiment 91: The recombinant DNA molecule of Embodiment 87 or 88, wherein the 5′ junctional cleavage sequence is a ribozyme sequence and the 3′ junctional cleavage sequence is a restriction enzyme recognition sequence.

Embodiment 92: The recombinant DNA molecule of Embodiment 91, wherein the 5′ ribozyme sequence is a hammerhead ribozyme sequence, a Pistol ribozyme sequence, or a modified Pistol ribozyme sequence.

Embodiment 93: The recombinant DNA molecule of Embodiment 91 or 92, wherein 3′ restriction enzyme recognition sequence is a Type IIS restriction enzyme recognition sequence.

Embodiment 94: The recombinant DNA molecule of Embodiment 93, wherein the Type IIS recognition sequence is a SapI recognition sequence.

Embodiment 95: The recombinant DNA molecule of Embodiment 87 or 88, wherein the 5′ junctional cleavage sequence is an RNAseH primer binding sequence and the 3′ junctional cleavage sequence is a restriction enzyme recognition sequence.

Embodiment 96: A method of producing the recombinant RNA molecule of any one of Embodiments 54-86, comprising in vitro transcription of the DNA molecule of any one of Embodiments 87-95 and purifying the resulting recombinant RNA molecule.

Embodiment 98: The method of Embodiment 96, wherein the recombinant RNA molecule comprises 5′ and 3′ ends that are native to the oncolytic virus encoded by the synthetic RNA viral genome.

Embodiment 99: A composition comprising an effective amount of the recombinant RNA molecule of any one of Embodiments 54-86, and a carrier suitable for administration to a mammalian subject.

Embodiment 100: A particle comprising the recombinant RNA molecule of any one of Embodiments 54-86.

Embodiment 101: The particle of Embodiment 100, wherein the particle is biodegradable.

Embodiment 102: The particle of Embodiment 101, wherein the particle is selected from the group consisting of a nanoparticle, an exosome, a liposome, and a lipoplex.

Embodiment 103: The particle of Embodiment 102, wherein the exosome is a modified exosome derived from an intact exosome or an empty exosome.

Embodiment 104: The particle of Embodiment 102, wherein the nanoparticle is a lipid nanoparticle (LNP) comprising cationic lipid, one or more helper lipids and a phospholipid-polymer conjugate.

Embodiment 105: The particle of Embodiment 104, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

Embodiment 106: The particle of Embodiment 104 or 105, wherein the helper lipid is selected from 1,2-distearoyl-sn-glycero-3-phosphocholine (DSPC); 1,2-dilauroyl-sn-glycero-3-phosphoethanolamine (DLPE); 1,2-dioteoyl-sn-glycero-3-phosphocholine (DOPC); 1,2-dioleoyl-sn-glycero-3-phosphoethanolamine (DOPE); and cholesterol.

Embodiment 107: The particle of Embodiment 104, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

Embodiment 108: The particle of Embodiment any one of Embodiments 104-106, wherein the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

Embodiment 109: The particle of any one of Embodiments 104-108, wherein the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

Embodiment 110: The particle of Embodiment 104, wherein the cationic lipid comprises COATSOME® SS-OC, wherein the one or more helper lipids comprise cholesterol (Chol) and DSPC, and wherein the phospholipid-polymer conjugate comprises DPG-PEG2000.

Embodiment 111: The particle of Embodiment 110, wherein the ratio of SS-OC:DSPC:Chol:DPG-PEG2K (as a percentage of total lipid content) is A:B:C:D, wherein A=40%-60%, B=10%-25%, C=20%-30%, and D 0%-3% and wherein A+B+C+D=100%.

Embodiment 112: The particle of Embodiment 110, wherein the ratio of SS-OC:DSPC:Chol:DPG-PEG2K (as a percentage of total lipid content) is A:B:C:D, wherein A=45%-50%, B=20%-25%, C=25%-30%, and D=0%-1% and wherein A+B+C+D=100%.

Embodiment 113: The particle of Embodiment 110, wherein the ratio of SS-OC:DSPC:Chol:DPG-PEG2K (as a percentage of total lipid content) is about 49:22:28.5:0.5.

Embodiment 114: The particle of Embodiment 110, wherein the ratio of SS-OC:DSPC:Chol:DPG-PEG2K (as a percentage of total lipid content) is A:B:C:D, wherein A=40%-60%, B=10%-30%, C=20%-45%, and D=0%-3% and wherein A+B+C+D100%.

Embodiment 114A: The particle of Embodiment 110, wherein the ratio of SS-OC:DSPC:Chol:DPG-PEG2K (as a percentage of total lipid content) is A:B:C:D, wherein A=40%-60%, B=10%-30%, C=25%-45%, and D=0%-3% and wherein A+B+C+D=100%.

Embodiment 114B: The particle of Embodiment 110, wherein the ratio of SS-OC:DSPC:Chol:DPG-PEG2K (as a percentage of total lipid content) is A:B:C:D, wherein A=45%-55%, B=10%-20%, C=30%-40%, and D=1%-2% and wherein A+B+C+D=100%.

Embodiment 115: The particle of Embodiment 110, wherein the ratio of SS-OC:DSPC:Chol:DPG-PEG2K (as a percentage of total lipid content) is A:B:C:D, wherein A=45%-50%, B=10%-15%, C=35%-40%, and D=1%-2% and wherein A+B+C+D=100%.

Embodiment 116: The particle of Embodiment 110, wherein the ratio of SS-OC:DSPC:Chol:DPG-PEG2K (as a percentage of total lipid content) is 49:11:38.5:1.5.

Embodiment 116A: The particle of Embodiment 110, wherein the ratio of SS-OC:DSPC:Chol:DPG-PEG2K (as a percentage of total lipid content) is about A:B:C:D, wherein A=45%-65%, B=5%-20%, C=20%-45%, and D=0%-3% and wherein A+B+C+D=100%.

Embodiment 116B: The particle of Embodiment 110, wherein the ratio of SS-OC:DSPC:Chol:DPG-PEG2K (as a percentage of total lipid content) is about A:B:C:D, wherein A=50%-60%, B=5%-15%, C=30%-45%, and D=0%-3% and wherein A+B+C+D=100%.

Embodiment 116C: The particle of Embodiment 110, wherein the ratio of SS-OC:DSPC:Chol:DPG-PEG2K (as a percentage of total lipid content) is about A:B:C:D, wherein A=55%-60%, B=5%-15%, C=30%-40%, and D=1%-2% and wherein A+B+C+D=100%.

Embodiment 116D: The particle of Embodiment 110, wherein the ratio of SS-OC:DSPC:Chol:DPG-PEG2K (as a percentage of total lipid content) is about A:B:C:D, wherein A=55%-60%, B=5%-10%, C=30%-35%, and D=1%-2% and wherein A+B+C+D=100%.

Embodiment 116E: The particle of Embodiment 110, wherein the ratio of SS-OC:DSPC:Chol:DPG-PEG2K (as a percentage of total lipid content) is 58:7:33.5:1.5.

Embodiment 117: The particle of any one of Embodiments 100-116E, wherein the LNP comprises a lipid formulation selected from Table 5.

Embodiment 118: The particle of Embodiment 104, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-alycero-3-phosphoethanolamine (DOPE).

Embodiment 119: The particle of Embodiment 104 or 118, further comprising a phospholipid-polymer conjugate, wherein the phospholipid-polymer conjugate is 1,2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

Embodiment 120: The particle of any one of Embodiments 104-119, wherein hyaluronan is conjugated to the surface of the LNP.

Embodiment 121: The particle of any one of Embodiments 104-119, further comprising a second recombinant RNA molecule encoding a payload molecule.

Embodiment 122: The particle of Embodiment 121, wherein the second recombinant RNA molecule is a replicon.

Embodiment 123: The particle of Embodiment 122, wherein the second recombinant RNA molecule is an alphavirus replicon.

Embodiment 124: A therapeutic composition comprising a plurality of lipid nanoparticles according to any one of Embodiments 104-123.

Embodiment 125: The therapeutic composition of Embodiment 124, wherein the plurality of LNPs have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

Embodiment 125A: The therapeutic composition of Embodiment 124, wherein the plurality of LNPs have an average size of about 50 nm to about 120 nm.

Embodiment 125B: The therapeutic composition of Embodiment 124, wherein the plurality of LNPs have an average size of about 50 nm, 60 nm, 70 nm, 80 nm, 90 nm, 100 nm, 110 nm, or about 120 mm.

Embodiment 125C: The therapeutic composition of Embodiment 124, wherein the plurality of LNPs have an average size of about 100 nm

Embodiment 126: The therapeutic composition of any one of Embodiments 124-125C, wherein the plurality of LNPs have an average zeta-potential of between about 40 mV and about −40 mV, about 20 mV to about −20 mV, about 10 mV to about −10 mV, about 5 mV to about −5 mV, or about 20 mV to about −40 mV.

Embodiment 127: The therapeutic composition of any one of Embodiments 124-125C, wherein the plurality of LNPs have an average zeta-potential of less than about −20 mV, less than about −30 mV, less than about −35 mV, or less than about −40 mV.

Embodiment 128: The therapeutic composition of any one of Embodiments 124-125C, wherein the plurality of LNPs have an average zeta-potential of between about −50 mV to about −20 mV, about −40 mV to about −20 mV, or about −30 mV to about −20 mV.

Embodiment 129: The therapeutic composition of any one of Embodiments 124-125C, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

Embodiment 130: The therapeutic composition of any one of Embodiments 124-129, wherein delivery of the composition to a subject delivers the encapsulated recombinant RNA molecule to a target cell, and wherein the encapsulated recombinant RNA molecule produces an infectious virus capable of lysing the target cell.

Embodiment 131: The therapeutic composition of Embodiment 130, wherein the composition is formulated for intravenous or intratumoral delivery.

Embodiment 132: The therapeutic composition of Embodiment 131, wherein the target cell is a cancerous cell.

Embodiment 133: An inorganic particle comprising the recombinant polynucleotide of any one of Embodiments 54-86.

Embodiment 134: The inorganic particle of Embodiment 133, wherein the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP).

Embodiment 135: The inorganic particle of Embodiment 133, further comprising a second recombinant RNA molecule encoding a payload molecule.

Embodiment 136: The particle of Embodiment 135, wherein the second recombinant RNA molecule is a replicon.

Embodiment 137: A composition comprising the inorganic particle of any one of Embodiments 133-136, wherein the average diameter of the particles is less than about 500 nm, is between about 50 nm and about 500 nm, is between about 250 nm and about 500 nm, or is about 350 nm.

Embodiment 138: A method of killing a cancerous cell comprising exposing the cancerous cell to the particle of any one of Embodiments 1-41, 100-123, or 133-136, the recombinant RNA molecule of any one of Embodiments 54-86, or compositions thereof, under conditions sufficient for the intracellular delivery of the particle to said cancerous cell, wherein the replication-competent virus produced by the encapsulated polynucleotide results in killing of the cancerous cell.

Embodiment 139: The method of Embodiment 138, wherein the replication-competent virus is not produced in non-cancerous cells.

Embodiment 140: The method of Embodiment 138 or 139, wherein the method is performed in vivo, in vitro, or ex vivo.

Embodiment 141: A method of treating a cancer in a subject comprising administering to a subject suffering from the cancer an effective amount of the particle of any one of Embodiments 1-41, 100-123, or 133-136, the recombinant RNA molecule of any one of claims 54-86, or compositions thereof.

Embodiment 142: The method of Embodiment 141, wherein the particle or composition thereof is administered intravenously, intranasally, as an inhalant, or is injected directly into a tumor.

Embodiment 143: The method of Embodiment 141 or 142, wherein the particle or composition thereof is administered to the subject repeatedly.

Embodiment 144: The method of any of Embodiments 141-143, wherein the subject is a mouse, a rat, a rabbit, a cat, a dog, a horse, a non-human primate, or a human.

Embodiment 145: The method of any of Embodiments 141-144, wherein the cancer is selected from lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, B-cell chronic lymphocytic leukemia, diffuse large B-cell lymphoma (DLBCL), sarcoma, neuroblastoma, rhabdomyosarcoma, medulloblastoma, a bladder cancer, and marginal zone lymphoma (MZL).

Embodiment 146: The method of Embodiment 145, wherein the lung cancer is small cell lung cancer or non-small cell lung cancer.

Embodiment 147: The method of Embodiment 145, wherein the liver cancer is hepatocellular carcinoma (HCC).

Embodiment 148: The method of Embodiment 145, wherein the prostate cancer is treatment-emergent neuroendocrine prostate cancer.

Embodiment 148A: The method of any one of Embodiments 141-148, wherein the cancer is a neuroendocrine cancer.

### EXAMPLES

The following examples are given for the purpose of illustrating various embodiments of the disclosure and are not meant to limit the present disclosure in any fashion. The present examples; along with the methods described herein are presently representative of preferred embodiments; are exemplary; and are not intended as limitations on the scope of the disclosure. Changes therein and other uses which are encompassed within the spirit of the disclosure as defined by the scope of the claims will occur to those skilled in the art.

### Example 1: Production of Infectious Picornavirus Virus from Recombinant RNA Molecules

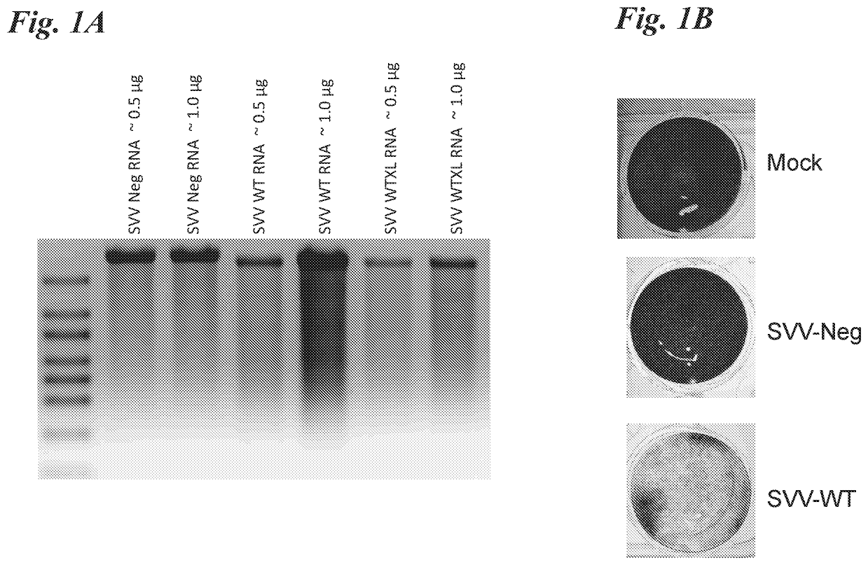

Experiments were performed to assess the ability to produce infectious SVV virus from recombinant RNA molecules. Briefly, RNA polynucleotides comprising SVV viral genomes were generated by T7 transcription in vitro and 293T cells were transfected with 1 μg of the SVV RNA constructs in Lipofectamine RNAiMax for 4 hours, cells were washed, and complete media was added to each well. Supernatants from transfected 293T were collected after 72 hours, syringe filtered with 0.45 μM filter and serially diluted onto NCI-H1299 cells. After 48 hours, supernatants were removed from the NCI-H1299 cultures and cells were stained with crystal violet to assess viral infectivity. As shown in FIG. 1B, RNA molecules comprising SVV-WT genomes produced active lytic virus.

In addition, supernatants of NCI-H1299 cells treated with 1 μg of SVV-WT RNA lipid, SVV-WT plasmid DNA, or SVV-Negative pDNA control were collected after 72 hours and serially diluted onto uninfected NCI-H1299 cells. Cell viability assays were performed according to standard protocols. As shown in FIG. 2, SVV-WT RNA/LNP are capable of producing infectious virus that results in tumor cell lysis in vitro.

### Example 2: Formulation of Lipid Nanoparticles for Intravenous Delivery of SVV-Encoding RNA

Recombinant RNA molecules comprising SVV genomes were formulated in lipid nanoparticles for delivery of the RNA in vivo.

Lipid nanoparticle production: The following lipids were used in formulation of lipid nanoparticles:

(a) D-Lin-MC3-DMA (MC3);

(b) N-(2,3-dioleoyloxy)propyl)-N,N,N-trimethylammonium chloride (DOTAP)

(c) COATSOME® SS-LC (former name: SS-18/4PE-13);

(d) COATSOME® SS-EC (former name: SS-33/4PE-15);

(e) COATSOME® SS-OC;

(f) COATSOME® SS-OP;

(g) Di((Z)-non-2-en-1-yl)9-((4-dimethylamino)butanoyl)oxy)heptadecanedioate (L-319)

(h) cholesterol;

(i) 1,2-distearoyl-sn-glycero-3-phosphocholine (DSPC);

(j) 1,2-dilauroyl-sn-glycero-3-phosphoethanolamine (DLPE);

(k) 1,2-dioleoyl-sn-glycero-3-phosphocholine (DOPC);

(l) 1,2-dioleoyl-sn-glycero-3-phosphoethanolamine (DOPE);

(m) 1,2-distearoyl-sn-glycero-3-phosphoethanolamine-N-[amino(polyethyleneglycol)-5000] (DSPE-PEG5K);

(n) 1,2-dipalmitoyl-rac-glycerol methoxypolyethylene glycol-2000 (DPG-PEG2K);

(o) 1,2-distearoyl-rac-glycero-3-methylpolyoxyethylene-2000 (DSG-PEG2K); and

(p) 1,2-dimyristoyl-rac-glycero-3-methylpolyoxyethylene-2000 (DMG-PEG2K).

Lipids were prepared in ethanol at various ratios shown below in Table 5. RNA lipid nanoparticles were then generated using microfluidic micromixture (Precision NanoSystems, Vancouver, BC) at a combined flow rate of 2 mL/min (0.5 mL/min for ethanol, lipid mix and 1.5 mL/min for aqueous buffer, RNA). The resulting particles were washed by tangential flow filtration with PBS containing Ca and Mg. Exemplary SVV LNP formulations are provided in Table 5. Unless otherwise indicated, each of the encapsulated RNA genomes were generated from IVT templates comprising a 5′ Hammerhead ribozyme and a 3′ Hepatitis delta ribozyme.

TABLE 5
Exemplary SVV RNA Lipid Nanopartides

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70001-5C | SVV- | PB, pH 5.8 | MC3 | Cholesterol | DSPC | DSPE-PEG5K | 7 |
| | WT | | (49%) | (39.8%) | (11%) | (0.2%) | |
| 70009-1.C | SVV- | PB, pH 5.8 | MC3 | Cholesterol | DSPC | DSPE-PEG5K | 7 |
| | Neg* | | (49%) | (39.8%) | (11%) | (0.2%) | |
| 70009-2.C | SVV- | PB, pH 5.8 | MC3 | Cholesterol | DSPC | DSPE-PEG5K | 7 |
| | WT | | (49%) | (39.8%) | (11%) | (0.2%) | |
| 70009-3.C | SVV- | PB, pH 5.8 | MC3 | Cholesterol | DSPC | DSPE-PEG5K | 7 |
| | S177A | | (49%) | (39.8%) | (11%) | (0.2%) | |
| 70032-1.C | SVV- | PB, pH 5.8 | MC3 | Cholesterol | DSPC | DSPE-PEG5K | 7 |
| | WT | | (49%) | (39.8%) | (11%) | (0.2%) | |
| 70032-2.C | SVV- | PB, pH 5.8 | MC3 | Cholesterol | DSPC | DMG-PEG2K | 7 |
| | WT | | (49%) | (38.5%) | (11%) | (1.5%) | |
| 70032-3.C | SVV- | PB, pH 5.8 | MC3 | Cholesterol | DSPC | DSPE-PEG5K | 5 |
| | WT | | (49%) | (39.8%) | (11%) | (0.2%) | |
| 70032-4.C | SVV- | PB, pH 5.8 | MC3 | Cholesterol | DSPC | DSPE-PEG5K | 3 |
| | WT | | (49%) | (39.8%) | (11%) | (0.2%) | |
| 70032-5.C | SVV- | PB, pH 7.4 | DOTAP | Cholesterol | DLPE | DSPE-PEG5K | 5.33 |
| | WT | | (50%) | (34.8%) | (15%) | (0.2%) | |
| 70032-6C | CVA21 | PB, pH 5.8 | MC3 | Cholesterol | DSPC | DSPE-PEG5K | 7 |
| | | | (49%) | (39.8%) | (11%) | (0.2%) | |
| 70041-2.C | SVV- | MB, pH 3.0 | SS-EC | Cholesterol | DSPC | DSPE-PEG5K | 7 |
| | WT | | (49%) | (39.8%) | (11%) | (0.2%) | |
| 70041-3.C | SVV- | MB, pH 3.0 | SS-LC | Cholesterol | DSPC | DSPE-PEG5K | 7 |
| | WT | | (49%) | (39.8%) | (11%) | (0.2%) | |
| 70041-4.C | SVV- | MB, pH 3.0 | SS-OC | Cholesterol | DSPC | DSPE-PEG5K | 7 |
| | WT | | (49%) | (39.8%) | (11%) | (0.2%) | |
| 70046-4.C | SVV- | MB, pH 3.0 | SS-EC | Cholesterol | DOPC | DSG-PEG5K | 9 |
| | WT | | (56%) | (28.0%) | (9%) | (7%) | |
| 70046-5.C | SVV- | MB, pH 3.0 | SS-LC | Cholesterol | DOPC | DSG-PEG5K | 9 |
| | WT | | (56%) | (28.0%) | (9%) | (7%) | |
| 70046-6.C | SVV- | MB, pH 3.0 | SS-OC | Cholesterol | DOPC | DSG-PEG5K | 9 |
| | WT | | (56%) | (28.0%) | (9%) | (7%) | |
| 70053-1.C | SVV- | PB, pH 5.8 | MC3 | Cholesterol | DSPC | DSPE-PEG5K | 7 |
| | WT | | (49%) | (39.8%) | (11%) | (0.2%) | |
| 70053-2.C | SVV- | MB, pH 3.0 | SS-LC | Cholesterol | DSPC | DSPE-PEG5K | 7 |
| | WT | | (49%) | (39.8%) | (11%) | (0.2%) | |
| 70059-1.C | SVV- | MB, pH 3.0 | SS-EC | Cholesterol | DSPC | DSPE-PEG5K | 7 |
| | WT | | (49%) | (39.8%) | (11%) | (0.2%) | |
| 70059-2.C | SVV- | MB, pH 3.0 | SS-EC | Cholesterol | DSPC | DSPE-PEG5K | 7 |
| | WT | | (49%) | (39.5%) | (11%) | (0.5%) | |
| 70059-3.C | SVV- | MB, pH 3.0 | SS-EC | Cholesterol | DSPC | DSPE-PEG5K | 7 |
| | WT | | (49%) | (39.0%) | (11%) | (1.0%) | |
| 70065-2.C | SVV- | MB, pH 3.0 | SS-LC | Cholesterol | DSPC | DMG-PEG2K | 7 |
| | WT | | (49%) | (39.5%) | (11%) | (0.5%) | |
| 70065-3.C | SVV- | MB, pH 3.0 | SS-LC | Cholesterol | DSPC | DMG-PEG2K | 7 |
| | WT | | (49%) | (39.0%) | (11%) | (1.0%) | |
| 70065-4.C | SVV- | MB, pH 3.0 | SS-LC | Cholesterol | DSPC | DMG-PEG2K | 7 |
| | WT | | (49%) | (38.5%) | (11%) | (1.5%) | |
| 70065-5.C | SVV- | MB, pH 3.0 | SS-LC | Cholesterol | DSPC | DMG-PEG5K | 7 |
| | WT | | (49%) | (39.5%) | (11%) | (0.5%) | |
| 70065-6.C | SVV- | MB, pH 3.0 | SS-LC | Cholesterol | DSPC | DMG-PEG5K | 7 |
| | WT | | (49%) | (39.0%) | (11%) | (1.0%) | |
| 70070-1.C | SVV- | PB, pH 5.8 | MC3 | Cholesterol | DSPC | DSPE-PEG5K | 7 |
| | WT | | (49%) | (39.8%) | (11%) | (0.2%) | |
| 70070-4.C | SVV- | PB, pH 5.8 | MC3 | Cholesterol | DSPC | DMG-PEG2K | 7 |
| | WT | | (49%) | (39.8%) | (11%) | (0.2%) | |
| 70070-5.C | SVV- | PB, pH 5.8 | MC3 | Cholesterol | DSPC | DMG-PEG2K | 7 |
| | WT | | (49%) | (38.0%) | (11%) | (2.0%) | |
| 70070-6.C | SVV- | PB, pH 5.8 | MC3 | Cholesterol | DSPC | DMG-PEG2K | 7 |
| | WT | | (49%) | (35.0%) | (11%) | (5.0%) | |
| 70077-3.C | SVV- | PB, pH 5.8 | MC3 | Cholesterol | DSPC | DSPE-PEG5K | 7 |
| | S177A | | (49%) | (39.8%) | (11%) | (0.2%) | |
| 70077-4.C | SVV- | MB, pH 3.0 | SS-OC | Cholesterol | DSPC | DSPE-PEG5K | 7 |
| | S177A | | (49%) | (39.8%) | (11%) | (0.2%) | |
| 70077-5.C | SVV- | MB, pH 3.0 | SS-OC | Cholesterol | DSPC | DSG-PEG2K | 7 |
| | S177A | | (49%) | (48.5%) | (2%) | (0.5%) | |
| 70077-6.C | SVV- | MB, pH 3.0 | SS-OC | Cholesterol | DSPC | DPG-PEG2K | 7 |
| | S177A | | (49%) | (48.0%) | (2%) | (1.0%) | |
| 70077-7.C | SVV- | MB, pH 3.0 | SS-OC | Cholesterol | DSPC | DMG-PEG2K | 7 |
| | S177A | | (49%) | (47.0%) | (2%) | (2.0%) | |
| 70077-8.C | SVV- | MB, pH 3.0 | SS-OC | Cholesterol | DSPC | DMG-PEG2K | 7 |
| | S177A | | (49%) | (38.5%) | (12%) | (0.5%) | |
| 70077-9.C | SVV- | MB, pH 3.0 | SS-OC | Cholesterol | DSPC | DSG-PEG2K | 7 |
| | S177A | | (49%) | (37.0%) | (12%) | (2.0%) | |
| 70077-10.C | SVV- | MB, pH 3.0 | SS-OC | Cholesterol | DSPC | DPG-PEG2K | 7 |
| | S177A | | (49%) | (28.5%) | (22%) | (0.5%) | |
| 70077-11.C | SVV- | MB, pH 3.0 | SS-OC | Cholesterol | DSPC | DMG-PEG2K | 7 |
| | S177A | | (49%) | (28.0%) | (22%) | (1.0%) | |
| 70087-1.C | SVV- | PB, pH 5.8 | MC3 | Cholesterol | DSPC | DSPE-PEG5K | 7 |
| | S177A | | (49%) | (39.8%) | (11%) | (0.2%) | |
| 70087-2.C | SVV- | MB, pH 3.0 | SS-OC | Cholesterol | DSPC | DPG-PEG2K | 7 |
| | S177A | | (60%) | (28.5%) | (11%) | (0.5%) | |
| 70087-3.C | SVV- | MB, pH 3.0 | SS-OC | Cholesterol | DSPC | DSG-PEG2K | 7 |
| | S177A | | (60%) | (28.5%) | (11%) | (0.5%) | |
| 70087-4.C | SVV- | MB, pH 3.0 | SS-OC | Cholesterol | DSPC | DMG-PEG2K | 7 |
| | S177A | | (60%) | (28.5%) | (11%) | (0.5%) | |
| 70087-5.C | SVV- | MB, pH 3.0 | SS-OC | Cholesterol | DSPC | DPG-PEG2K | 7 |
| | S177A | | (60%) | (27.0%) | (11%) | (1.5%) | |
| 80010-1.C | SVV- | PB, pH 5.8 | MC3 | Cholesterol | DSPC | DSPE-PEG5K | 7 |
| | S177A | | (49%) | (39.8%) | (11%) | (0.2%) | |
| 80010-2.C | SVV- | MB, pH 3.0 | SS-OC | Cholesterol | DSPC | DPG-PEG2K | 7 |
| | S177A | | (49%) | (28.5%) | (22%) | (0.5%) | |
| 80010-3.C | SVV- | MB, pH 3.0 | SS-OC | Cholesterol | DSPC | DMG-PEG2K | 7 |
| | S177A | | (49%) | (28.5%) | (22%) | (0.5%) | |
| 80010-4.C | SVV- | MB, pH 3.0 | SS-OC | Cholesterol | DSPC | DSG-PEG2K | 7 |
| | S177A | | (49%) | (28.5%) | (22%) | (0.5%) | |
| 80010-5.C | SVV- | MB, pH 3.0 | SS-OC | Cholesterol | DSPC | DPG-PEG2K | 7 |
| | S177A | | (49%) | (28.0%) | (22%) | (1.0%) | |
| 80016-1.C | SVV- | PB, pH 5.8 | MC3 | Cholesterol | DSPC | DSPE-PEG5K | 7 |
| | S177A | | (49%) | (39.8%) | (11%) | (0.2%) | |
| 80016-2.C | SVV- | MB, pH 3.0 | MC3 | Cholesterol | DSPC | DPG-PEG2K | 7 |
| | S177A | | (49%) | (28.5%) | (22%) | (0.5%) | |
| 80016-3.C | SVV- | MB, pH 3.0 | SS-OC | Cholesterol | DSPC | DPG-PEG2K | 5.5 |
| | S177A | | (49%) | (26.5%) | (22%) | (0.5%) | |
| 80016-6.C | SVV- | MB, pH 3.0 | SS-OC | Cholesterol | DSPC | DPG-PEG2K | 7 |
| | S177A | | (49%) | (28.5%) | (22%) | (0.5%) | |
| 80016-7.C | SVV- | MB, pH 3.0 | SS-OC | Cholesterol | DSPC | DPG-PEG2K | 7 |
| | S177A | | (49%) | (27.5%) | (22%) | (1.5%) | |
| 80016-9.C | SVV- | MB, pH 3.0 | L-319 | Cholesterol | DSPC | DMG-PEG2K | 7 |
| | S177A | | (49%) | (28.5%) | (22%) | (0.2%) | |
| 80016-10.C | SVV- | MB, pH 3.0 | L-319 | Cholesterol | DSPC | DMG-PEG2K | 7 |
| | S177A | | (49%) | (27.5%) | (22%) | (1.5%) | |
| 80016-11.C | SVV- | MB, pH 3.0 | L-319 | Cholesterol | DSPC | DMG-PEG2K | 7 |
| | S177A | | (49%) | (26.5%) | (22%) | (2.5%) | |
| 80033-1.C | SVV- | MB, pH 3.0 | SS-OC | Cholesterol | DSPC | DPG-PEG2K | 7 |
| | S177A | | (49%) | (28.5%) | (22%) | (0.5%) | |
| 80033-2.C | SVV- | MB, pH 3.0 | SS-LC | Cholesterol | DSPC | DPG-PEG2K | 7 |
| | S177A | | (49%) | (28.5%) | (22%) | (0.5%) | |
| 80033-3.C | SVV- | MB, pH 3.0 | SS-OP | Cholesterol | DSPC | DPG-PEG2K | 7 |
| | S177A | | (49%) | (28.5%) | (22%) | (0.5%) | |
| 80048-1.C | SVV- | MB, pH 3.0 | SS-OC | Cholesterol | DSPC | DPG-PEG2K | 9 |
| | S177A | | (49%) | (35.5%) | (15%) | (0.5%) | |
| 80048-2.C | SVV- | MB, pH 3.0 | SS-OC | Cholesterol | DSPC | DPG-PEG2K | 5 |
| | S177A | | (49%) | (28.5%) | (22%) | (0.5%) | |
| 80048-3.C | SVV- | MB, pH 3.0 | SS-OC | Cholesterol | DSPC | DPG-PEG2K | 7 |
| | S177A | | (49%) | (21.5%) | (29%) | (0.5%) | |
| 80048-4.C | SVV- | MB, pH 3.0 | SS-OC | Cholesterol | DOPE | DPG-PEG2K | 5 |
| | S177A | | (49%) | (21.5%) | (29%) | (0.5%) | |
| 80048-5.C | SVV- | MB, pH 3.0 | SS-OC | Cholesterol | DOPE | DPG-PEG2K | 9 |
| | S177A | | (49%) | (28.5%) | (22%) | (0.5%) | |
| 80048-6.C | SVV- | MB, pH 3.0 | SS-OC | Cholesterol | DOPE | DPG-PEG2K | 7 |
| | S177A | | (49%) | (35.5%) | (15%) | (0.5%) | |
| 80048-7.C | SVV- | MB, pH 3.0 | SS-OC | Cholesterol | DLPE | DPG-PEG2K | 5 |
| | S177A | | (49%) | (35.5%) | (15%) | (0.5%) | |
| 80048-8.C | SVV- | MB, pH 3.0 | SS-OC | Cholesterol | DLPE | DPG-PEG2K | 7 |
| | S177A | | (49%) | (28.5%) | (22%) | (0.5%) | |
| 80048-9.C | SVV- | MB, pH 3.0 | SS-OC | Cholesterol | DLPE | DPG-PEG2K | 9 |
| | S177A | | (49%) | (21.5%) | (29%) | (0.5%) | |
| 80059.1.C | SVV- | MB, pH 3.0 | SS-OC | Cholesterol | DLPE | DPG-PEG2K | 7 |
| | Neg* | | (49%) | (28.5%) | (22%) | (0.5%) | |
| 80059-2.C | SVV- | MB, pH 3.0 | SS-OC | Cholesterol | DLPE | DPG-PEG2K | 7 |
| | S177A | | (49%) | (28.5%) | (22%) | (0.5%) | |
| 80130-1.C | SVV- | MB, pH 3.0 | SS-OC | Cholesterol | DSPC | DPG-PEG2K | 7 |
| | WT | | (49%) | (28.5%) | (22%) | (0.5%) | |
| 80130-2.C | SVV- | MB, pH 3.0 | SS-OC | Cholesterol | DSPC | DPG-PEG2K | 7 |
| | IRES2 | | (49%) | (28.5%) | (22%) | (0.5%) | |
| 80130-3.C | SVV- | MB, pH 3.0 | SS-LC | Cholesterol | DSPC | DPG-PEG2K | 7 |
| | WT | | (49%) | (28.5%) | (22%) | (0.5%) | |
| | Pistol 1/ | | | | | | |
| | SapI | | | | | | |
| 80139-1.C | SVV- | MB, pH 3.0 | SS-OC | Cholesterol | DSPC | DPG-PEG2K | 7 |
| | WT | | (49%) | (28.5%) | (22%) | (0.5%) | |

*SVV-neg constructs were generated from IVT templates without ribozymes

Analysis of physical characteristics of lipid nanoparticles: Physical characteristics of lipid nanoparticles in Table 5 were evaluated before and after tangential flow filtration. Particle size distribution and zeta potential measurements were determined by light scattering using a Malvern Nano-ZS Zetasizer (Malvern Instruments Ltd, Worcestershire, UK). Size measurements were performed in HBS at pH 7.4 and zeta potential measurements were performed in 0.01 M HBS at pH 7.4. Percentage of RNA entrapment was measured by Ribogreen assay. Lipid nanoparticles that showed greater than 80 percent RNA entrapment were tested in vivo.

The physical characteristics of various lipid nanoparticle formulations are shown in Table 6 below.

TABLE 6
Physical Properties of SVV RNA Lipid Nanoparticles

| Formulation | | Z-Average | | | |
|---|---|---|---|---|---|
| ID | RNA | (d · nm) | PdI | ZP (mV) | EE (%) |
| 70001-5.C | SVV-WT | 145 | 0.17 | 0.2 | |
| 70009-1.C | SVV-Neg | 207 | 0.09 | −0.7 | |
| 70009-2.C | SVV-WT | 226 | 0.10 | −2.8 | |
| 70009-3.C | SVV-S177A | 232 | 0.13 | −1.9 | |
| 70032-1.C | SVV-WT | 148 | 0.15 | 1.4 | |
| 70032-2.C | SVV-WT | 99 | 0.36 | 1.4 | |
| 70032-3.C | SVV-WT | 169 | 0.14 | −1.6 | |
| 70032-4.C | SVV-WT | 208 | 0.07 | 2.0 | |
| 70032-5.C | SVV-WT | 383 | 0.22 | −8.8 | |
| 70032-6.C | CVA21 | 189 | 0.25 | −1.0 | |
| 70041-2.C | SVV-WT | 104 | 0.06 | −2.1 | |
| 70041-3.C | SVV-WT | 124 | 0.05 | 0.5 | |
| 70041-4.C | SVV-WT | 134 | 0.04 | −1.9 | |
| 70046-4.C | SVV-WT | 235 | 0.10 | −1.0 | |
| 70046-5.C | SVV-WT | 127 | 0.05 | −3.0 | |
| 70046-6.C | SVV-WT | 131 | 0.07 | −0.9 | |
| 70053-1.C | SVV-WT | 216 | 0.10 | −0.4 | |
| 70053-2.C | SVV-WT | 141 | 0.10 | −13.6 | |
| 70059-1.C | SVV-WT | 166 | 0.22 | −1.8 | 81 |
| 70059-2.C | SVV-WT | 116 | 0.24 | −1.4 | 92 |
| 70059-3.C | SVV-WT | 133 | 0.22 | −1.2 | 88 |
| 70065-2.C | SVV-WT | 153 | 0.21 | −0.8 | 84 |
| 70065-3.C | SVV-WT | 181 | 0.23 | 0.0 | 62 |
| 70065-4.C | SVV-WT | 197 | 0.34 | −2.0 | 44 |
| 70065-5.C | SVV-WT | 158 | 0.13 | −1.3 | 30 |
| 70065-6.C | SVV-WT | 144 | 0.15 | −3.0 | 32 |
| 70070-1.C | SVV-WT | 237 | 0.10 | −0.8 | 80 |
| 70070-4.C | SVV-WT | 442 | 0.22 | 0.0 | 87 |
| 70070-5.C | SVV-WT | 132 | 0.29 | −0.4 | 92 |
| 70070-6.C | SVV-WT | 87 | 0.18 | 0.0 | 68 |
| 70077-3.C | SVV-S177A | 222 | 0.13 | −1.6 | 93 |
| 70077-4.C | SVV-S177A | 160 | 0.06 | −3.8 | 82 |
| 70077-5.C | SVV-S177A | 292 | 0.19 | −25.4 | 39 |
| 70077-6.C | SVV-S177A | 205 | 0.09 | −12.3 | 45 |
| 70077-7.C | SVV-S177A | 133 | 0.08 | −7.5 | 41 |
| 70077-8.C | SVV-S177A | 158 | 0.10 | −5.0 | 90 |
| 70077-9.C | SVV-S177A | 128 | 0.12 | −2.4 | 79 |
| 70077-10.C | SVV-S177A | 110 | 0.14 | −3.9 | 97 |
| 70077-11.C | SVV-S177A | 132 | 0.19 | −5.2 | 83 |
| 70087-1.C | SVV-S177A | 224 | 0.14 | −13.2 | 87 |
| 70087-2.C | SVV-S177A | 154 | 0.08 | −15.6 | 86 |
| 70087-3.C | SVV-S177A | 149 | 0.03 | −10.0 | 88 |
| 70087-4.C | SVV-S177A | 145 | 0.08 | −5.4 | 89 |
| 70087-5.C | SVV-S177A | 148 | 0.05 | −2.6 | 68 |
| 80010-1.C | SVV-S177A | 235 | 0.09 | −2.7 | 82 |
| 80010-2.C | SVV-S177A | 91 | 0.05 | −4.6 | 93 |
| 80010-3.C | SVV-S177A | 93 | 0.11 | −8.6 | 92 |
| 80010-4.C | SVV-S177A | 110 | 0.03 | −4.2 | 94 |
| 80010-5.C | SVV-S177A | 99 | 0.09 | −4.1 | 95 |
| 80016-1.C | SVV-S177A | 231.5 | 0.12 | −2.2 | 91 |
| 80016-2.C | SVV-S177A | 131.0 | 0.18 | −6.5 | 96 |
| 80016-3.C | SVV-S177A | 84.7 | 0.14 | −13.4 | 83 |
| 80016-6.C | SVV-S177A | 94.9 | 0.14 | −12.1 | 93 |
| 80016-7.C | SVV-S177A | 92.1 | 0.18 | −19.6 | 92 |
| 80016-9.C | SVV-S177A | 155.0 | 0.17 | −9.4 | 22 |
| 80016-10.C | SVV-S177A | 108.6 | 0.19 | −9.2 | 34 |
| 80016-11.C | SVV-S177A | 123.8 | 0.18 | −8.9 | 19 |
| 80033-1.C | SVV-S177A | 115 | 0.22 | −14.3 | 88 |
| 80033-2.C | SVV-S177A | 143 | 0.23 | −15.9 | 92 |
| 80033-3.C | SVV-S177A | 105 | 0.22 | −12.5 | 95 |
| 80048-1.C | SVV-S177A | 106.4 | 0.13 | −15.7 | 90 |
| 80048-2.C | SVV-S177A | 96.4 | 0.15 | −19.4 | 89 |
| 80048-3.C | SVV-S177A | 99.9 | 0.20 | −6.5 | 95 |
| 80048-4.C | SVV-S177A | 219.2 | 0.11 | −6.3 | 29 |
| 80048-5.C | SVV-S177A | 257.0 | 0.09 | −2.9 | 20 |
| 80048-6.C | SVV-S177A | 257.7 | 0.12 | −6.6 | 28 |
| 80048-7.C | SVV-S177A | 186.1 | 0.14 | −7.9 | 61 |
| 80048-8.C | SVV-S177A | 172.1 | 0.20 | −6.7 | 72 |
| 80048-9.C | SVV-S177A | 153.9 | 0.22 | −5.4 | 84 |
| 80059-1.C | SVV-Neg | 84.39 | 0.12 | −7.7 | 91 |
| 80059-2.C | SVV-S177A | 84.33 | 0.11 | −8.3 | 93 |
| 80130-1.C | SVV-WT | 98.8 | 0.15 | 2.7 | 97 |
| 80130-2.C | SVV-IRES2 | 91.9 | 0.12 | 2.8 | 98 |
| 80130-3.C | SVV-Pistol1/ SapI | 112.6 | 0.21 | 8.3 | 98 |
| 80139-1.C | SVV-WT | 111.5 | 0.22 | 1.6 | 95 |

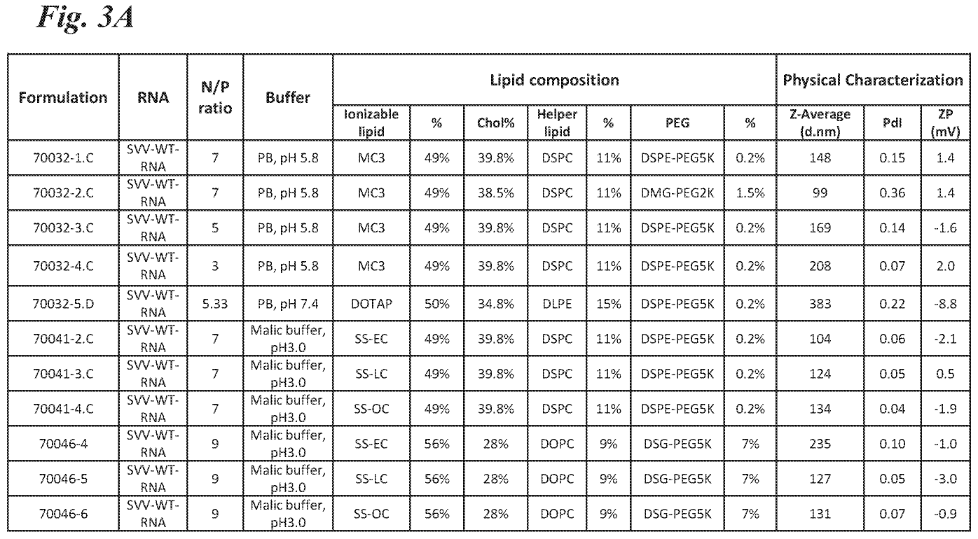

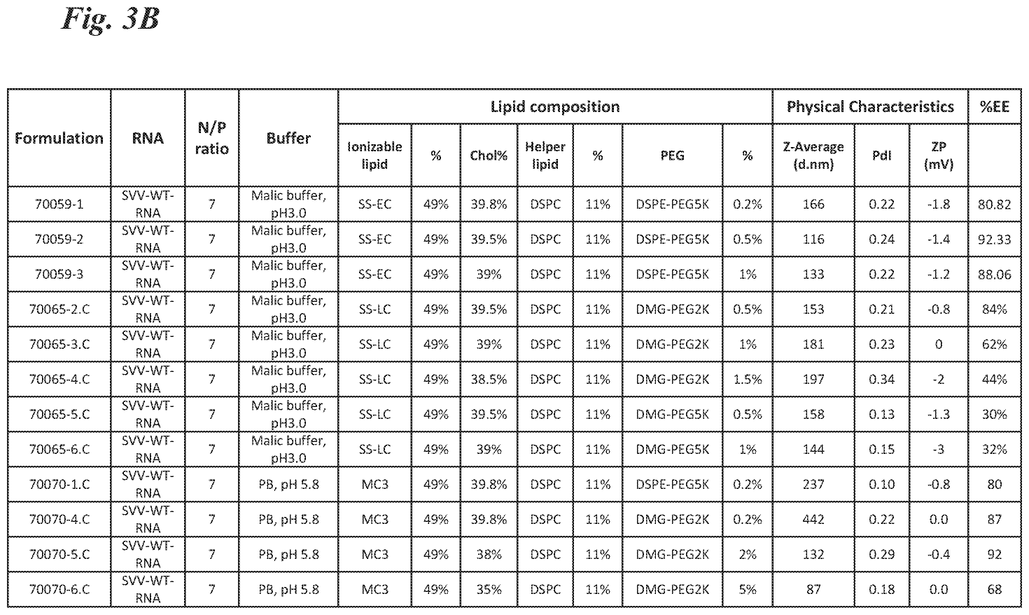

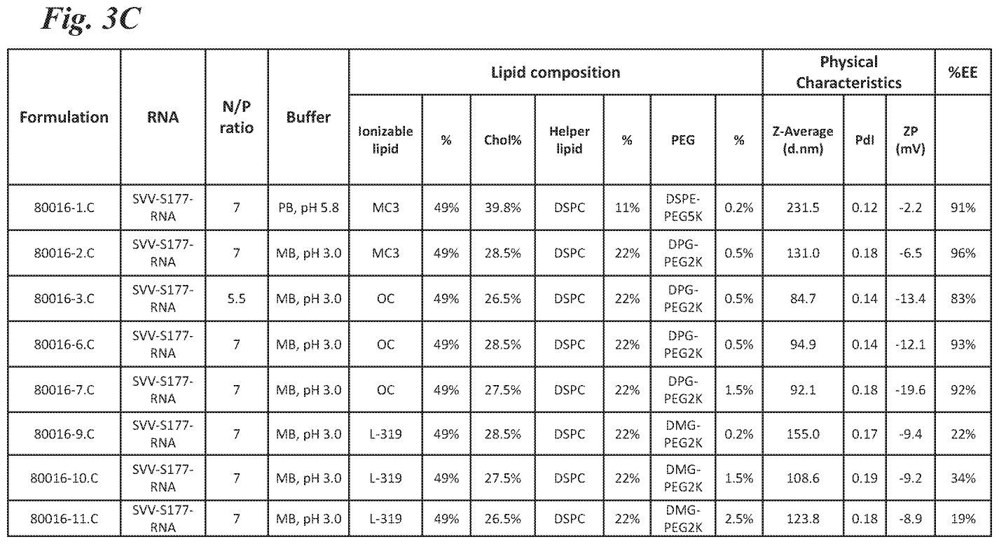

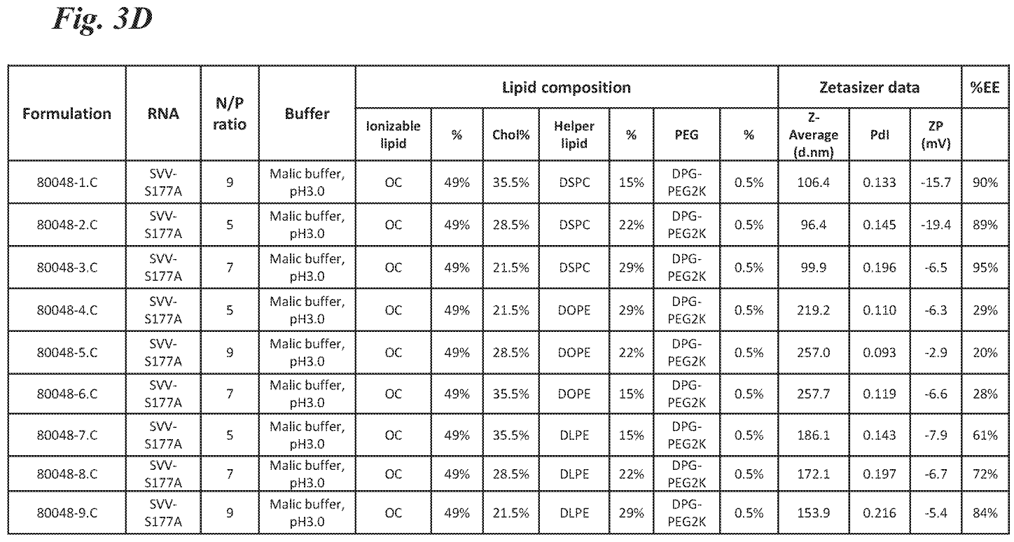

As shown in FIG. 3A-FIG. 3D and Table 6, variation in lipid composition and lipid ratio alters nanoparticle size and/or RNA entrapment. Lipid nanoparticles that showed greater than 80 percent RNA entrapment were tested in vivo in Examples 4-18 described below.

### Example 3: Lipid Nanoparticles Comprising SVV-Encoding RNA Produce Infectious Virus and Inhibit Tumor growth In Vivo

Experiments were performed to determine the ability of lipid nanoparticles comprising SVV-WT RNA to produce infectious virus in vivo in order to inhibit tumor growth in mice.

SVV RNA lipid nanoparticle production, formulation, and analysis of the physical characteristics were performed as described in Example 3 and are summarized in Table 7 below.

TABLE 7
SVV RNA Lipid Nanoparticle Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70001-5C | SVV- | PB, pH 5.8 | MC3 | Cholesterol | DSPC | DSPE-PEG5K | 7 |
| | WT | | (49%) | (39.8%) | (11%) | (0.2%) | |

The ability of SVV RNA lipid nanoparticles to inhibit tumor growth was evaluated using the H1299 xenograft model. Briefly, 5×10⁶ NCI-H1299 cells were suspended in 0.1 mL of serum-free DPBS and Matrigel (1:1 v/v) and inoculated subcutaneously in the right flank of 8-week week old female athymic nude mice (Charles River Laboratories). Mice were randomly assigned into experimental groups and treatments started when median tumor size reaches approximately 150 mm³ (120-180 mm³ range).

Two doses of lipid nanoparticles containing SVV-WT RNA (5 μg/dose) were intravenously administered on day 1 and day 8. Tumor volume was measured 3 times per week using electronic calipers. On day 16, tumors were harvested for assessment of infectious virus.

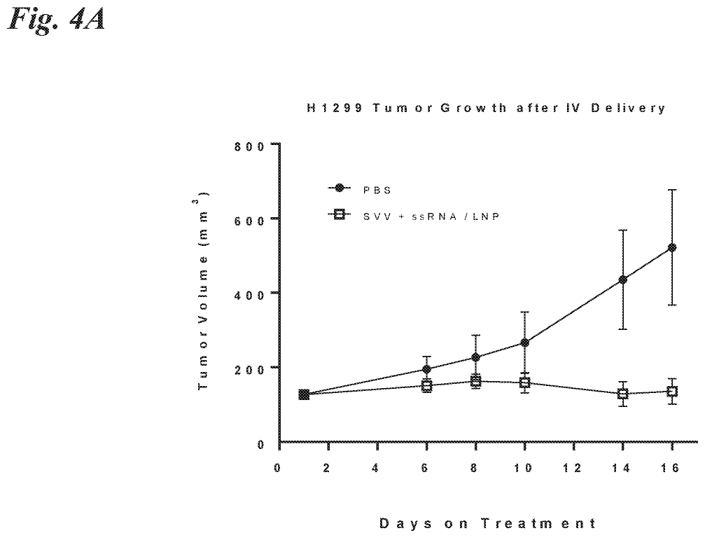

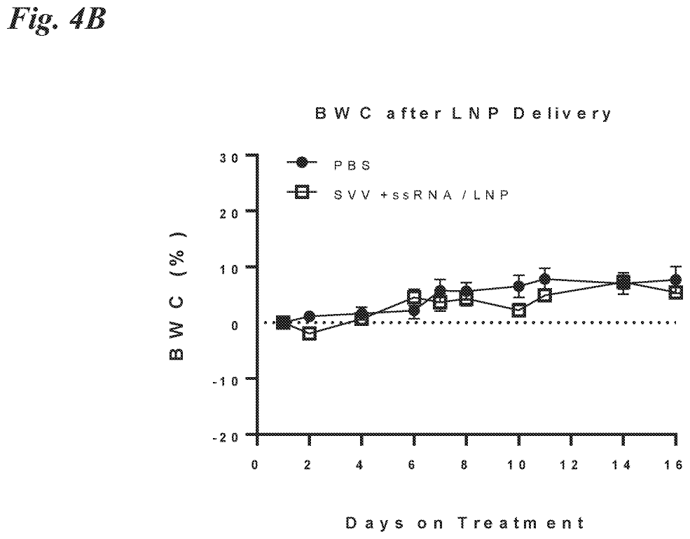

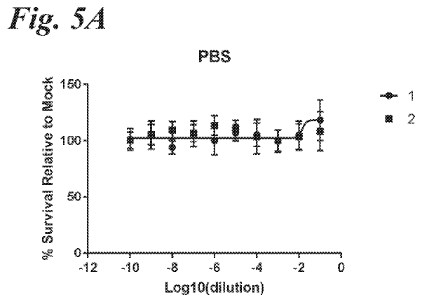

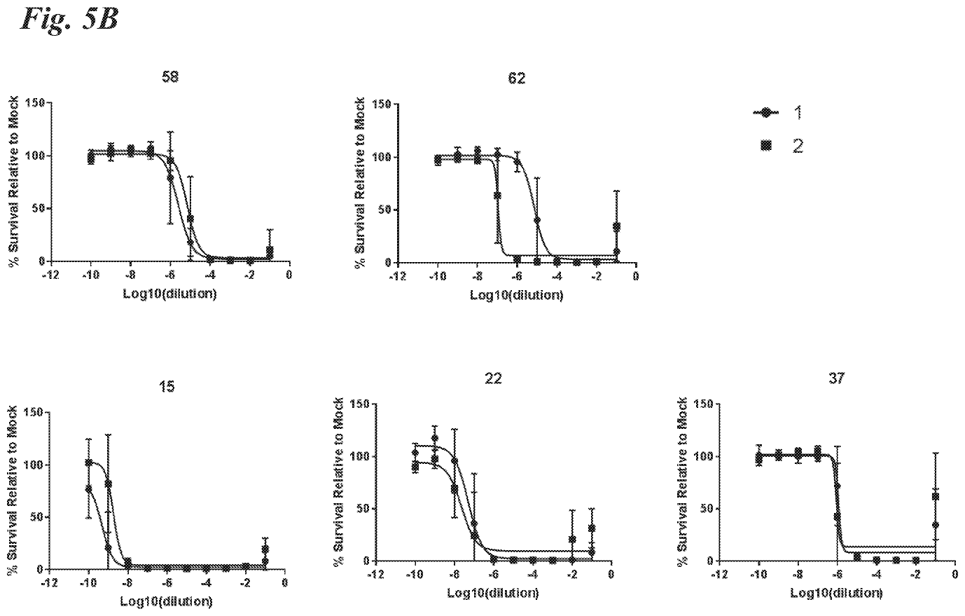

As shown in FIG. 4A, mice treated with SVV-WT lipid nanoparticles showed a significant reduction in tumor growth compared to mice treated with PBS (two-way RM ANOVA, p<0.0.001). Further, as shown in FIG. 4B, treatment with SVV-WT lipid nanoparticles did not affect body weight, suggesting that the lipid nanoparticles were non-toxic when administered intravenously. FIG. 5A and FIG. 5B shows the recovery of infectious SVV from tumors after intravenous dosing of SVV-WT lipid nanoparticles.

### Example 4: Particles Comprising Gain-of-Function SVV-Encoding RNA Inhibited Tumor Growth

Experiments were performed to determine the ability of lipid nanoparticles comprising a SVV-encoding RNA molecule with a gain-of-function mutation (SVV-S177A) to produce infectious virus in vivo in order to inhibit tumor growth in mice.

SVV RNA lipid nanoparticle production, formulation, and analysis of the physical characteristics were performed as described in Example 3 and are summarized in Table 8 below.

TABLE 8
SVV RNA Lipid Nanoparticle Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70009-1.C | SVV- | PB, pH 5.8 | MC3 | Cholesterol | DSPC | DSPE-PEG5K | 7 |
| | Neg | | (49%) | (39.8%) | (11%) | (0.2%) | |
| 70009-2.C | SVV- | PB, pH 5.8 | MC3 | Cholesterol | DSPC | DSPE-PEG5K | 7 |
| | WT | | (49%) | (39.8%) | (11%) | (0.2%) | |
| 70009-3.C | SVV- | PB, pH 5.8 | MC3 | Cholesterol | DSPC | DSPE-PEG5K | 7 |
| | S177A | | (49%) | (39.8%) | (11%) | (0.2%) | |

The ability of SVV RNA lipid nanoparticles to inhibit tumor growth was evaluated using the H1299 xenograft model, which is further described in Example 3. Briefly, 5×10⁶ NCI-H1299 cells were subcutaneously inoculated in the right flank of 8-week-old female athymic nude mice. Mice were randomly assigned into 4 experimental groups: (i) PBS only, (ii) SVV-Negative (SVV-Neg, formulation ID: 70009-1.C), (iii) SVV (wild-type, formulation ID: 70009-2.C), and (iv) SVV-S177A (formulation ID: 70009-3.C). SVV-Neg lipid nanoparticles was comprised of RNA molecules unable to replicate, but similar in size to SVV-WT and SVV-S177A RNA. When median tumor size reached approximately 150 mm³, lipid nanoparticles (5 μg/dose) were intravenously administered on day 1, followed by additional treatments on days 6, 11, and 16. Tumor volume was measured 3 times per week using electronic calipers. On day 22, mice were sacrificed and tumor and liver tissue were harvested to determine the presence of replicating, infectious virus by measuring the presence of minus-strand SVV RNA (a surrogate marker for replicating SVV) using qRT-PCR.

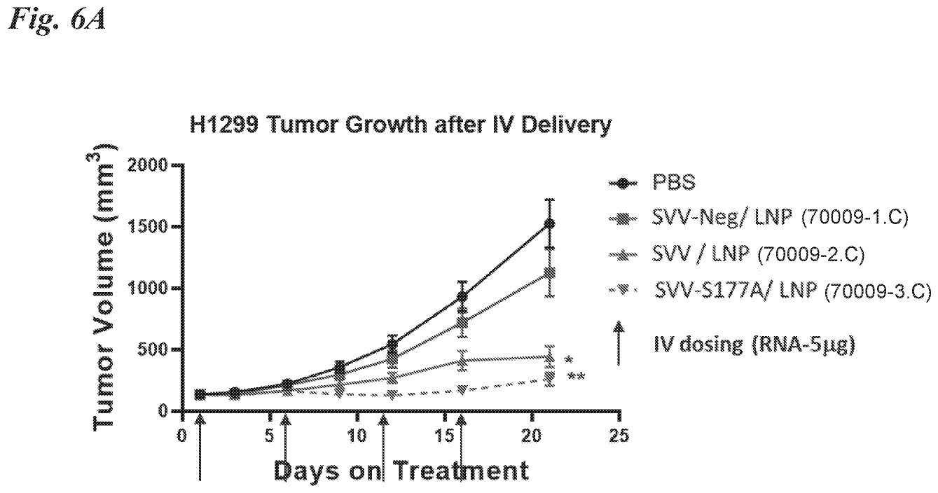

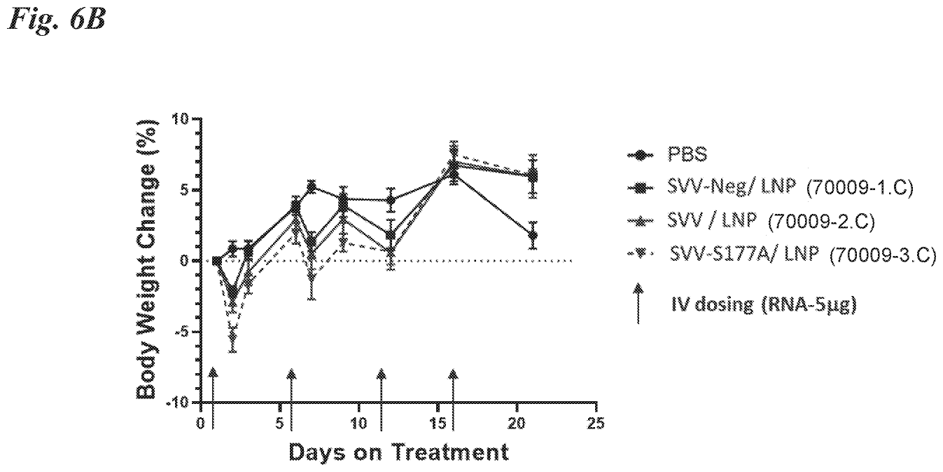

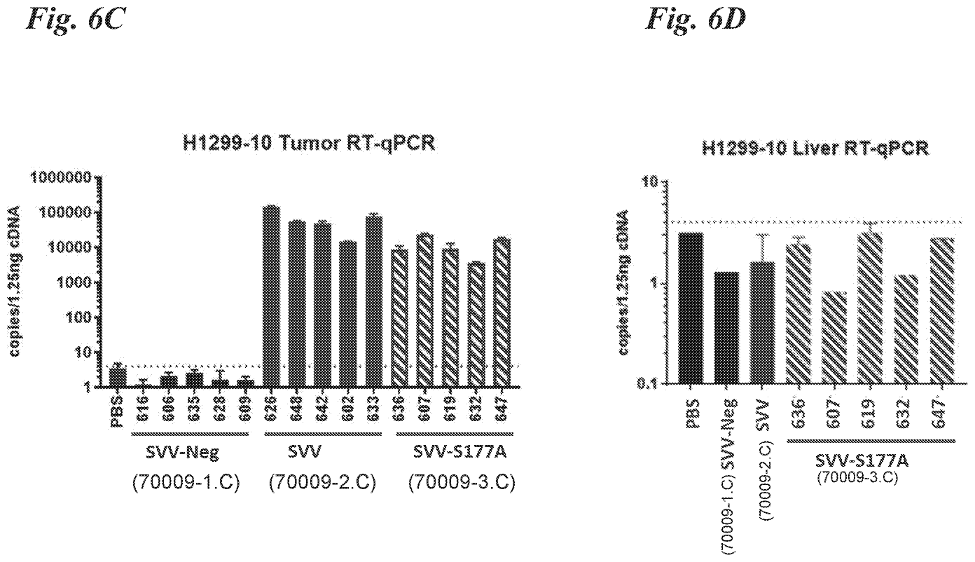

As shown in FIG. 6A, mice treated with SVV-WT or SVV-S177A lipid nanoparticles significantly inhibited tumor growth compared to mice treated with SVV-Neg lipid nanoparticles or PBS (p<0.05, Two-way ANOVA). Administration of SVV lipid nanoparticles had minimal impact on body weight throughout the course of the study (FIG. 6B), suggesting that the lipid nanoparticles comprising SVV-encoding RNA molecules were non-toxic and well-tolerated.

Active replication of SVV was detected in tumor tissue of mice treated with lipid nanoparticles comprising SVV-WT or SVV-S177A RNA molecules (FIG. 6C). Importantly, active viral replication did not occur in liver tissue of any treatment group, including mice that received lipid nanoparticles capable of producing infectious virus in tumor tissues (FIG. 6D).

### Example 5: Lipid Nanoparticles Comprising SVV-Encoding RNA Molecules Inhibit Tumor Growth

Experiments were performed to determine the ability of lipid nanoparticles comprising SVV-encoding RNA molecules to produce infectious virus and inhibit tumor growth in vivo.

SVV RNA lipid nanoparticle production, formulation, and analysis of the physical characteristics are described in Example 2 and in Table 9 below.

TABLE 9
SVV RNA Lipid Nanopartide Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70053-1.C | SVV- | PB, pH 5.8 | MC3 | Cholesterol | DSPC | DSPE-PEG5K | 7 |
| | WT | | (49%) | (39.8%) | (11%) | (0.2%) | |
| 70053-2.C | SVV- | MB, pH 3.0 | SS-LC | Cholesterol | DSPC | DSPE-PEG5K | 7 |
| | WT | | (49%) | (39.8%) | (11%) | (0.2%) | |
| 70059-1.C | SVV- | MB, pH 3.0 | SS-EC | Cholesterol | DSPC | DSPE-PEG5K | 7 |
| | WT | | (49%) | (39.8%) | (11%) | (0.2%) | |
| 70059-2.C | SVV- | MB, pH 3.0 | SS-EC | Cholesterol | DSPC (11%) | DSPE-PEG5K | 7 |
| | WT | | (49%) | (39.5%) | | (0.5%) | |

The ability of SVV RNA lipid nanoparticles to inhibit tumor growth was evaluated using the H1299 xenograft model, which is further described in Example 4. Briefly, 5×10⁶ H1299 cells were subcutaneously inoculated in the right flank of athymic nude mice. When median tumor size reached approximately 150 mm³, mice were intravenously administered two 5 μg doses of SVV lipid nanoparticles comprising MC-3, SS-LC, or SS-EC ionizable lipids (formulations shown in Table 9 above) on day 1 and day 6. Tumor volume was measured 3 times per week using electronic calipers. On day 12, tumor tissue was harvested and analyzed for the presence of minus-strand SVV RNA (a surrogate marker for replicating SVV) sing qRT-PCR.

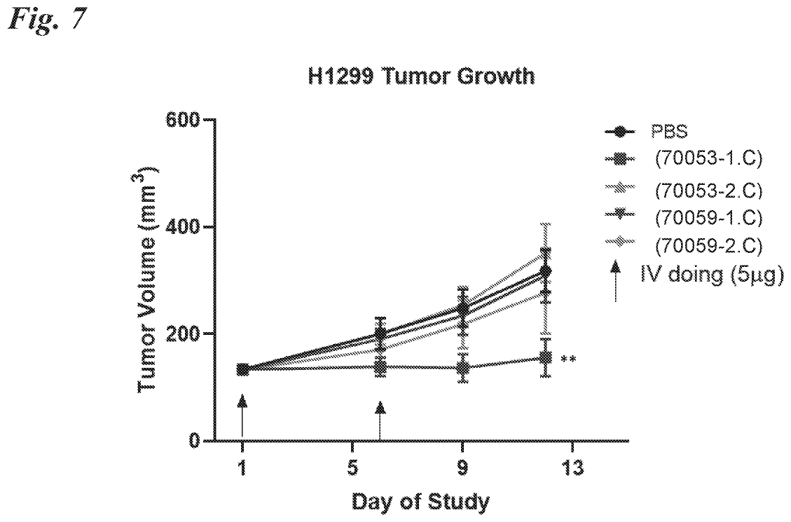

As shown in FIG. 7, mice treated with MC3-based lipid nanoparticles (formulation ID: 70053-1.C) demonstrated significant inhibition of tumor growth compared to mice treated with PBS (F test to compare variances **p<0.01). However, SS-LC-based or SS-EC-based lipid nanoparticles (formulation IDs: 70053-2.C, 70059-1.C or 70059-2.C) had no inhibitory effect on tumor growth. These data suggest that MC3-based lipid nanoparticles deliver SVV-encoding RNA molecules to tumor tissue resulting in production of infectious virus and tumor lysis in vivo.

### Example 6: In Vivo Efficacy of Lipid Nanoparticles Comprising SVV-Encoding RNA in Small Cell Lung Cancer

Experiments were performed to determine the ability of lipid nanoparticles comprising SVV-encoding RNA molecules to produce infectious virus and inhibit small cell lung cancer (SCLC) growth in vivo.

SVV RNA lipid nanoparticle production, formulation, and analysis of the physical characteristics are described in Example 2 and in Table 10 below.

TABLE 10
SVV RNA Lipid Nanoparticle Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70087-1.C | SVV- | PB, pH 5.8 | MC3 | Cholesterol | DSPC | DSPE-PEG5K | 7 |
| | S177A | | (49%) | (39.8%) | (11%) | (0.2%) | |

In vivo efficacy of SVV-encoding RNA lipid nanoparticles on SCLC was tested using the H82 xenograft model. Briefly, NCI-H82 cells (1×10⁶ cells/0.1 mL in a 1:1 mixture of serum-free PBS and Matrigel®) were subcutaneously inoculated in the right flank of 8-week-old female athymic nude mice (Charles River Laboratories). Mice began treatment when median tumor size reached approximately 150 mm³ (120-180 mm³ range), and were either intravenously administered 10 μg of SVV-WT lipid nanoparticles (formulation ID: 70087-1C) on days 1, 6, 11 and 16, or intratumorally administered 1 μg of SVV-WT RNA formulated with Lipofectamine RNAiMax (positive control) on days 1, and 4. Tumor volume was measured 3 times per week using electronic calipers.

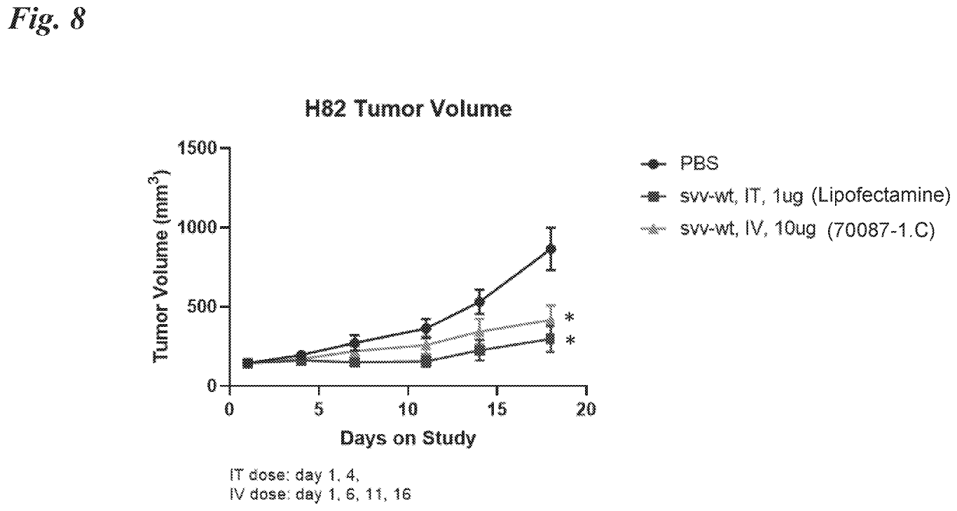

As shown in FIG. 8, mice treated with SVV-WT lipid nanoparticles or Lipofectamine-formulated SVV-WT RNA showed significant inhibition of tumor growth compared to mice treated with PBS (two-way ANOVA p<0.05). These results indicate that either intravenous administration of lipid nanoparticles comprising SVV-encoding RNA molecules or intratumoral administration of Lipofectamine-formulated SVV-encoding RNA molecules effectively initiates viral replication in tumor tissue resulting in tumor cell lysis.

### Example 7: Variation on Lipid Composition Can Alter Anti-Tumor Activity of SVV-Encoding RNA Nanoparticles

Experiments were performed to determine whether lipid composition of nanoparticles comprising SVV-encoding RNA molecules affects viral replication and anti-tumor activity in vivo.

SVV RNA lipid nanoparticle production, formulation, and analysis of physical characteristics are described in Example 2 and in Table 11 below.

TABLE 11
SVV RNA Lipid Nanoparticle Formulations

| Formulation | | Ionizable | | Helper | PEGylated | |
|---|---|---|---|---|---|---|
| ID | RNA | lipid | Cholesterol | Lipid | lipid (%) | N:P |
| 70077-3.C | SVV- | MC3 | Cholesterol | DSPC | DSPE-PEG5K | 7 |
| | S177A | (49%) | (39.8%) | (11%) | (0.2%) | |
| 70077-4.C | SVV- | SS-OC | Cholesterol | DSPC | DSPE-PEG5K | 7 |
| | S177A | (49%) | (39.8%) | (11%) | (0.2%) | |
| 70077-8.C | SVV- | SS-OC | Cholesterol | DSPC | DMG-PEG2K | 7 |
| | S177A | (49%) | (38.5%) | (12%) | (0.5%) | |
| 70077-10.C | SVV- | SS-OC | Cholesterol | DSPC | DPG-PEG2K | 7 |
| | S177A | (49%) | (28.5%) | (22%) | (0.5%) | |
| 70077-11.C | SVV- | SS-OC | Cholesterol | DSPC | DMG-PEG2K | 7 |
| | S177A | (49%) | (28.0%) | (22%) | (1.0%) | |

The ability of SVV RNA lipid nanoparticles to inhibit tumor growth was evaluated using the H1299 xenograft model, which is further described in Example 4. Briefly, 5×10⁶ NCI-H1299 cells were subcutaneously inoculated in the right flank of athymic nude mice. When median tumor size reached approximately 150 mm³, mice were intravenously administered 5 μg of lipid nanoparticles (formulations shown in Table 11 above) on days 1 and 6. Tumor volume was measured 3 times per week using electronic calipers. On day 12, tumor tissue was harvested and analyzed for the presence of minus-strand SVV RNA (a surrogate marker for replicating SVV) using qRT-PCR.

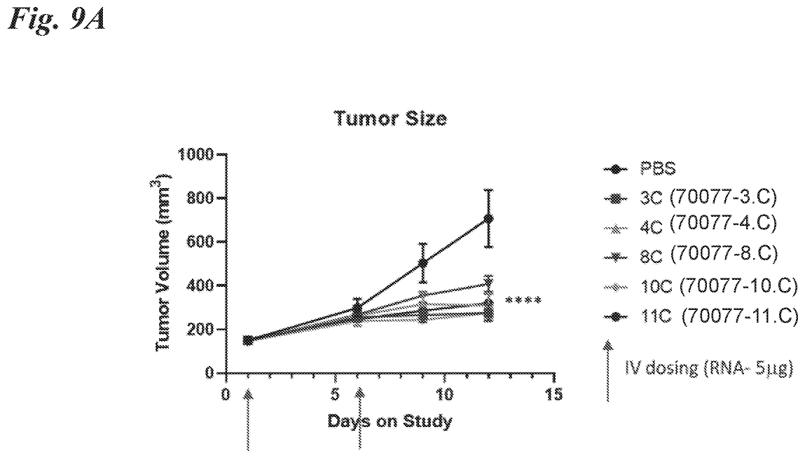

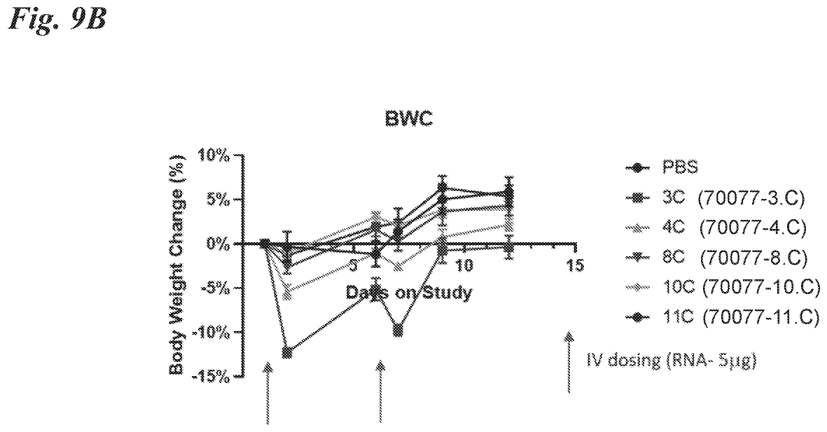

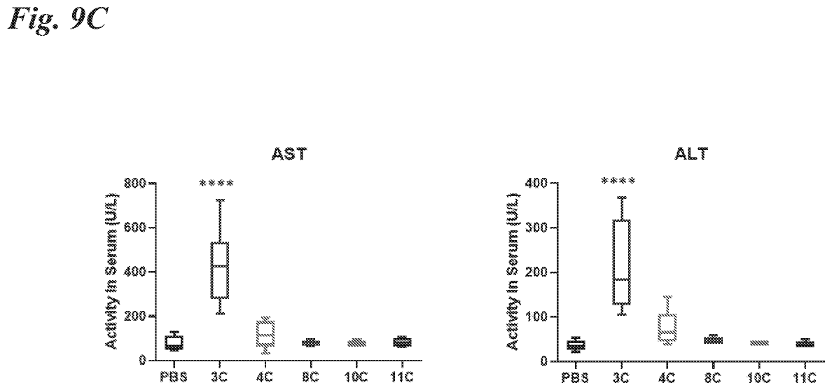

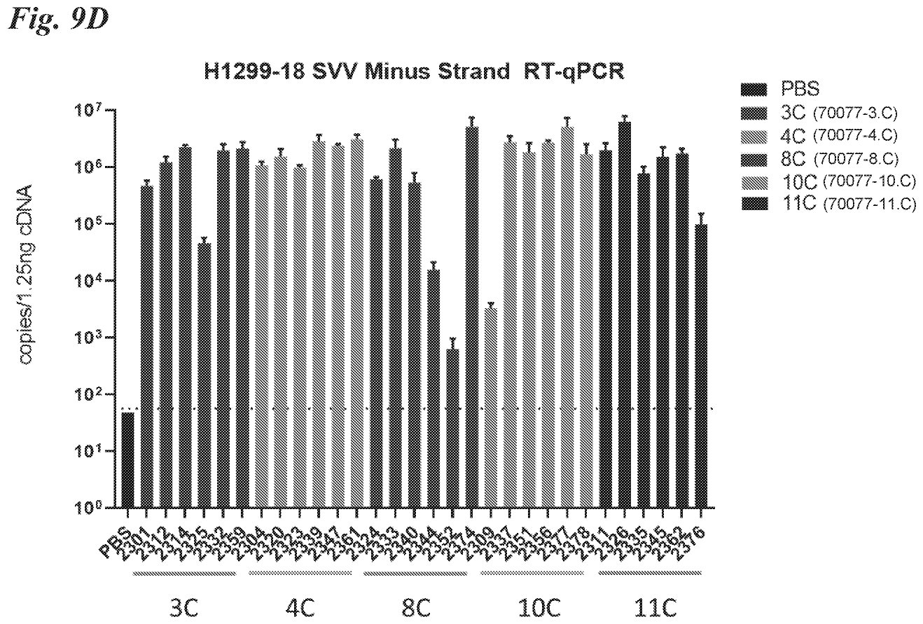

As shown in FIG. 9A, mice treated with both MC3-based and OC-based lipid nanoparticles (formulation IDs: 70077-3.C, 70077-4.C, 70077-8.C, 70077-10.C, and 70077-11.C) significantly inhibited tumor growth compared to mice treated with PBS (two-way ANOVA, Tukey's multiple comparison test, ****: p<0.0001 vs. PBS). Tumor tissue from mice treated with different lipid nanoparticle formulations showed the presence of minus-strand SVV RNA, confirming that intravenous administration of SVV-WT RNA lipid nanoparticles induced viral replication in tumor tissue (FIG. 9D). The MC3-based lipid nanoparticles caused weight loss and an increase in the liver enzymes AST and ALT, while the OC-based lipid nanoparticles did not affect these parameters (FIG. 9B and FIG. 9C). Collectively, these data indicated that the OC-based lipid nanoparticle formulations significantly inhibited tumor growth and were well-tolerated in mice.

### Example 8: PEG Composition Alters Lipid Nanoparticle Anti-Tumor Activity

Experiments were performed to determine whether PEG composition of lipid nanoparticles comprising SVV-encoding RNA molecules affects viral replication and anti-tumor activity in vivo.

SVV RNA lipid nanoparticle production, formulation, and analysis of physical characteristics are described in Example 2 and in Table 12 below.

TABLE 12
SVV RNA Lipid Nanoparticles Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70087-1.C | SVV- | PB, pH 5.8 | MC3 | Cholesterol | DSPC | DSPE-PEG5K | 7 |
| | S177A | | (49%) | (39.8%) | (11%) | (0.2%) | |
| 70087-2.C | SVV- | MB, pH 3.0 | SS-OC | Cholesterol | DSPC | DPG-PEG2K | 7 |
| | S177A | | (60%) | (28.5%) | (11%) | (0.5%) | |
| 70087-3.C | SVV- | MB, pH 3.0 | SS-OC | Cholesterol | DSPC | DSG-PEG2K | 7 |
| | S177A | | (60%) | (28.5%) | (11%) | (0.5%) | |
| 70087-4.C | SVV- | MB, pH 3.0 | SS-OC | Cholesterol | DSPC | DMG-PEG2K | 7 |
| | S177A | | (60%) | (28.5%) | (11%) | (0.5%) | |

The ability of SVV RNA lipid nanoparticles to inhibit tumor growth was evaluated using the H1299 xenograft model, which is further described in Example 4. Briefly, 5×10⁶ NCI-H1299 cells were subcutaneously inoculated in the right flank of athymic nude mice. When median tumor size reached approximately 150 mm³, mice were intravenously administered two doses of lipid nanoparticles containing SVV-encoding RNA molecules (5 μg/dose) on day 1, 6, 11 and day 16. Tumor volume was measured 3 times per week using electronic calipers.

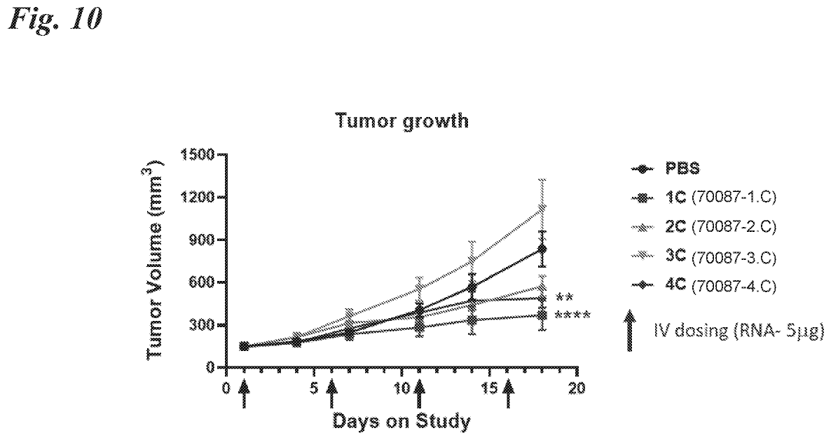

As shown in FIG. 10, mice treated with formulation 70087-1.C and 70087-4.C (Table 12) demonstrated significant inhibition of tumor growth compared to mice treated with PBS (two-way ANOVA, Tukey's multiple comparison test, ****: p<0.0001 and ***:p<0.001 vs. PBS). Formulations 70087-2.C and 70087-3. C only differ from 70087-4.C in the type of PEGylated lipid utilized in the formulation (Table 12). These findings suggest that the type of PEGylated lipid can have a significant impact on the anti-tumor activity of SVV-encoding RNA nanoparticles.

### Example 9: PEG Composition Alters OC-Based Lipid Nanoparticle Anti-Tumor Activity

Experiments were performed to determine whether PEG composition of lipid nanoparticles comprising SVV-encoding RNA molecules affects viral replication and anti-tumor activity in vivo.

SVV RNA lipid nanoparticle production, formulation, and analysis of physical characteristics are described in Example 2 and in Table 13 below.

TABLE 13
SVV RNA Lipid Nanoparticles Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 80010-1.C | SVV- | PB, pH 5.8 | MC3 | Cholesterol | DSPC | DSPE-PEG5K | 7 |
| | S177A | | (49%) | (39.8%) | (11%) | (0.2%) | |
| 80010-2.C | SVV- | MB, pH 3.0 | SS-OC | Cholesterol | DSPC | DPG-PEG2K | 7 |
| | S177A | | (49%) | (28.5%) | (22%) | (0.5%) | |
| 80010-3.C | SVV- | MB, pH 3.0 | SS-OC | Cholesterol | DSPC | DMG-PEG2K | 7 |
| | S177A | | (49%) | (28.5%) | (22%) | (0.5%) | |
| 80010-4.C | SVV- | MB, pH 3.0 | SS-OC | Cholesterol | DSPC | DSG-PEG2K | 7 |
| | S177A | | (49%) | (28.5%) | (22%) | (0.5%) | |
| 80010-5.C | SVV- | MB, pH 3.0 | SS-OC | Cholesterol | DSPC | DPG-PEG2K | 7 |
| | S177A | | (49%) | (28.0%) | (22%) | (1.0%) | |

The ability of SVV RNA lipid nanoparticles to inhibit tumor growth was evaluated using the H1299 xenograft model, which is further described in Example 4. Briefly, 5×10⁶ NCI-H1299 cells were subcutaneously administered to the right flank of athymic nude mice. When median tumor size reached approximately 150 mm³, mice were intravenously administered 5 μg of SVV RNA lipid nanoparticles containing either DSPE-PEG5K, or DPG-, DMG-, or DSG-PEG2K (formulations shown in Table 13). Tumor tissue was collected from mice 72 hours after treatment and analyzed for the presence of minus-strand SVV RNA (a surrogate marker for replicating SVV) using qRT-PCR.

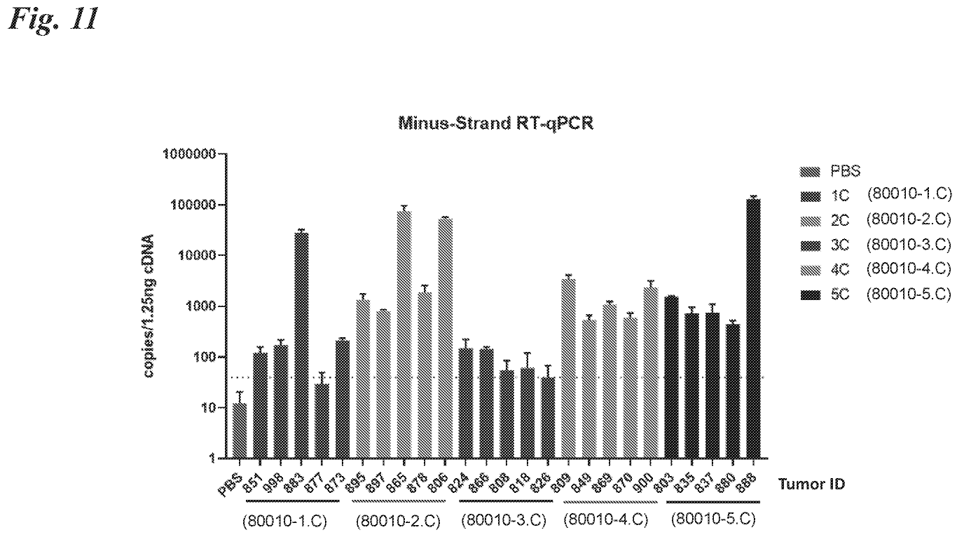

As shown in FIG. 11, the PEG type used in the formulation can alter the ability of the nanoparticle to efficiently deliver to the tumor tissue the SVV genome. Tumor of mice treated with formulations 80010-2.C, 80010-3.C, and 80010-4.C shown greater numbers of SVV minus (−) strand or SVV replication. Formulation 80010-2.C, 80010-3.C, 80010-4.C, and 80010-5.C only differ among themselves in the type and percentage of lipid-PEG utilized in the formulation. This demonstrated the selection of PEG type and the percentage used can have a significant impact the biological activity of these nanoparticles.

### Example 10: Ionizable Lipid Composition Alters Lipid Nanoparticle Anti-Tumor Activity

Experiments were performed to determine whether ionizable lipid composition affects the anti-tumor activity of lipid nanoparticles comprising SVV-encoding RNA molecules in vivo.

A description of lipid nanoparticle production, formulation, and analysis of the physical characteristics of SVV RNA lipid nanoparticles are described in Example 2 and in Table 14 below.

TABLE 14
SVV RNA Lipid Nanopartide Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 80033-1.C | SVV- | MB, pH 3.0 | SS-OC | Cholesterol | DSPC | DPG-PEG2K | 7 |
| | S177A | | (49%) | | (22%) | (0.5%) | |
| 80033-2.C | SVV- | MB, pH 3.0 | SS-LC | Cholesterol | DSPC | DPG-PEG2K | 7 |
| | S177A | | (49%) | (28.5%) | (22%) | (0.5%) | |
| 80033-3.C | SVV- | MB, pH 3.0 | SS-OP | Cholesterol | DSPC | DPG-PEG2K | 7 |
| | S177A | | (49%) | (28.5%) | (22%) | (0.5%) | |

The ability of ionizable lipid composition of SVV RNA nanoparticles to inhibit tumor growth was evaluated using the H1299 xenograft model, which is further described in Example 4. Briefly, 5×10⁶ NCI-H1299 cells were subcutaneously administered to the right flank of athymic nude mice. When median tumor size reached approximately 150 mm³, mice were intravenously administered 1 mg/kg of SVV-S177A RNA lipid nanoparticles comprising SS-OC (formulation ID: 80033-1.C), SS-LC (formulation ID: 80033-2.C), or SS-OP (formulation ID: 80033-3.C) ionizable lipids on days 1 and 8 of the study. Tumor volume was measured 3 times per week using electronic calipers.

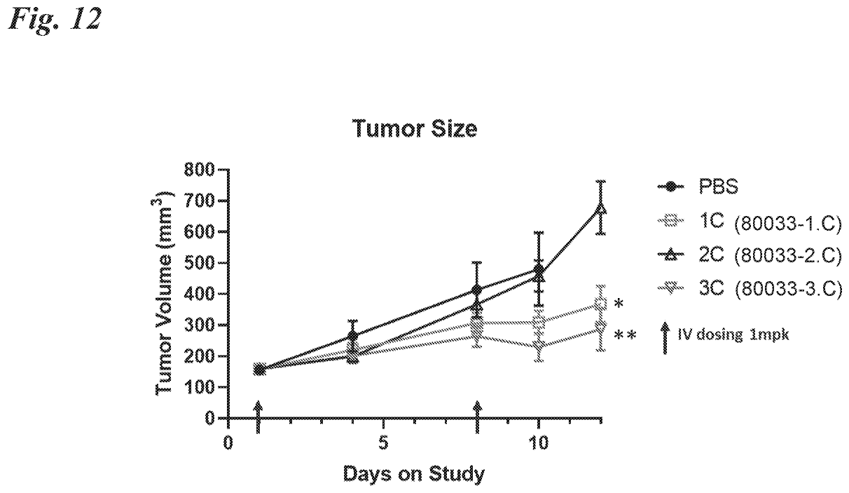

As shown in FIG. 12, mice treated with SS-OC-based or SS-OP based lipid nanoparticles showed significant inhibition in tumor growth compared to mice treated with PBS or SS-LC-based lipid nanoparticles (two-way ANOVA, Tukey's multiple comparison test, *p<0.05, **p<0.001 vs. PBS). These results demonstrate that ionizable lipid composition affects anti-tumor activity of SVV RNA lipid nanoparticles in vivo.

### Example 11: In Vivo Efficacy of Lipid Nanoparticles Comprising SVV-Encoding RNA Molecules in Small Cell Lung Cancer

Experiments were performed to determine the ability of lipid nanoparticles comprising SVV-encoding RNA molecules to produce infectious virus and inhibit small cell lung cancer (SCLC) growth in vivo.

SVV RNA lipid nanoparticle production, formulation, and analysis of the physical characteristics are described in Example 2 and in Table 15 below.

TABLE 15
SVV-RNA Lipid Nanopartide Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 80059.1.C | SVV- | MB, pH 3.0 | SS-OC | Cholesterol | DLPE | DPG-PEG2K | 7 |
| | Neg | | (49%) | (28.5%) | (22%) | (0.5%) | |
| 80059-2.C | SVV- | MB, pH 3.0 | SS-OC | Cholesterol | DLPE | DPG-PEG2K | 7 |
| | S177A | | (49%) | (28.5%) | (22%) | (0.5%) | |

In vivo efficacy of SVV-encoding RNA lipid nanoparticles on SCLC was tested using the H446 xenograft model. Briefly, NCI-H446 cells (5×10⁶ cells/0.1 mL in a 1:1 mixture of serum-free PBS and Matrigel®) were subcutaneously inoculated in the right flank of 8-week-old female athymic nude mice (Charles River Laboratories). When median tumor size reached approximately 150 mm³ (120-180 mm³ range), mice were intravenously administered 1 mg/kg of SVV-Neg (formulation ID: 80059-1.C) or SVV-S177A (formulation ID: 80059-2.C) RNA lipid nanoparticles or PBS on days 1, 8, and 15. Tumor volume was measured 3 times per week using electronic calipers. On day 22, tumors were harvested and analyzed for the presence of minus-strand SVV RNA (a surrogate marker for replicating SVV) using qRT-PCR.

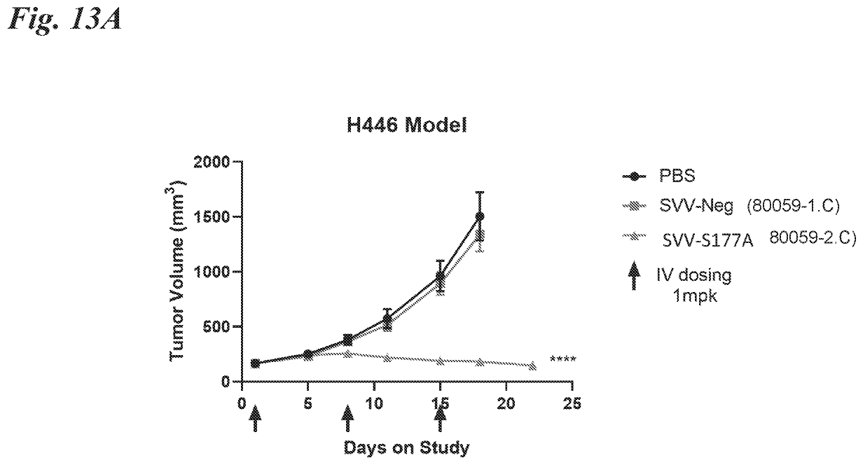

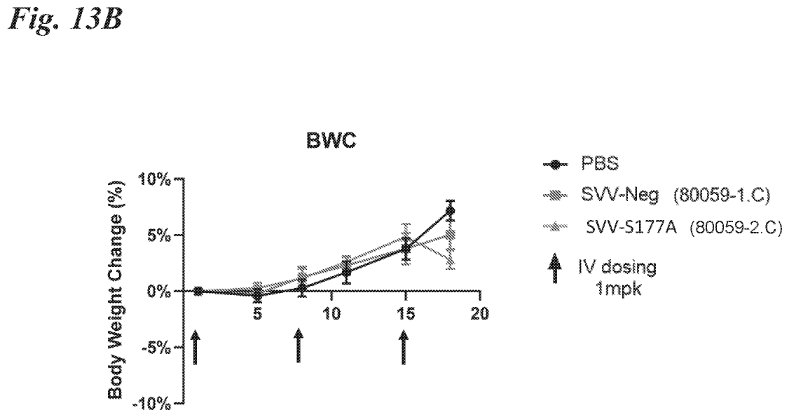

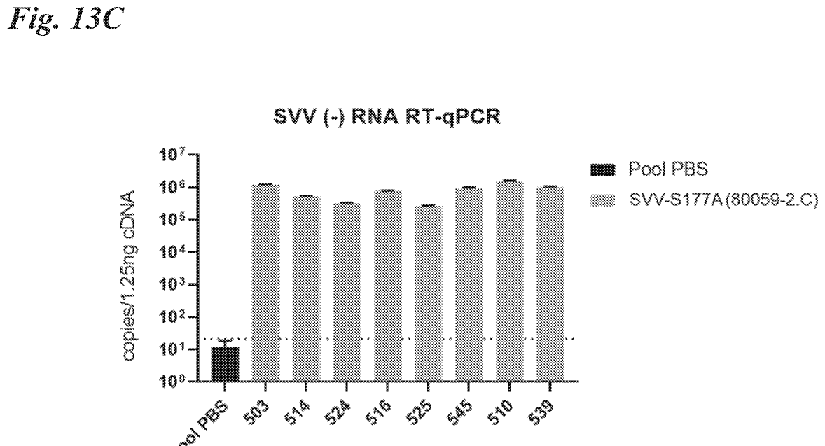

As shown in FIG. 13A, mice treated with SVV-S177A lipid nanoparticles demonstrated significant inhibition of tumor growth compared to mice treated with SVV-Neg lipid nanoparticles or PBS (two-way ANOVA, p<0.0.001). Importantly, intravenous administration of SVV-encoding RNA lipid nanoparticles had no effect on body weight, suggesting that these agents were non-toxic and well-tolerated (FIG. 13B). Tumor tissue from mice treated with SVV-S177A RNA lipid nanoparticles showed the presence of minus-strand SVV RNA, indicating that intravenous administration of SVV-S177A RNA lipid nanoparticles induced viral replication in tumor tissue (FIG. 13C). These results demonstrate that the systemic administration of SVV-S177A RNA lipid nanoparticles leads to SVV replication and lysis of SCLC cells in vivo.

### Example 12: In Vivo Efficacy of SVV-IRES2 RNA Lipid Nanoparticles on Tumor Growth

Experiments were performed to determine whether lipid nanoparticles containing SVV-IRES2 RNA could inhibit tumor growth in vivo.

SVV RNA lipid nanoparticle production, formulation and analysis is described in Example 2 and in Table 16 below.

TABLE 16
SVV RNA Lipid Nanopartide Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 80130-1.C | SVV- | MB, pH 3.0 | SS-OC | Cholesterol | DSPC | DPG-PEG2K | 7 |
| | WT | | (49%) | (28.5%) | (22%) | (0.5%) | |
| 80130-2.C | SVV- | MB, pH 3.0 | SS-OC | Cholesterol | DSPC | DPG-PEG2K | 7 |
| | IRES2 | | (49%) | (28.5%) | (22%) | (0.5%) | |

The ability of SVV-WT and SVV-IRES2 lipid nanoparticles to inhibit tumor growth was evaluated using the H1299 xenograft model, which is further described in Example 4. Briefly, 5×10⁶ NCI-H1299 cells were subcutaneously administered to the right flank of athymic nude mice. When median tumor size reached approximately 150 mm³, mice were intravenously administered 0.2 mg/kg of lipid nanoparticles containing SVV-WT (formulation ID: 80130-1.C) or SVV-IRES2 (formulation ID: 80130-2.C) RNA molecules (day 1). A subsequent dose of PBS or lipid nanoparticles was administered on day 8 of the study. Tumor volume was measured 3 times per week using electronic calipers.

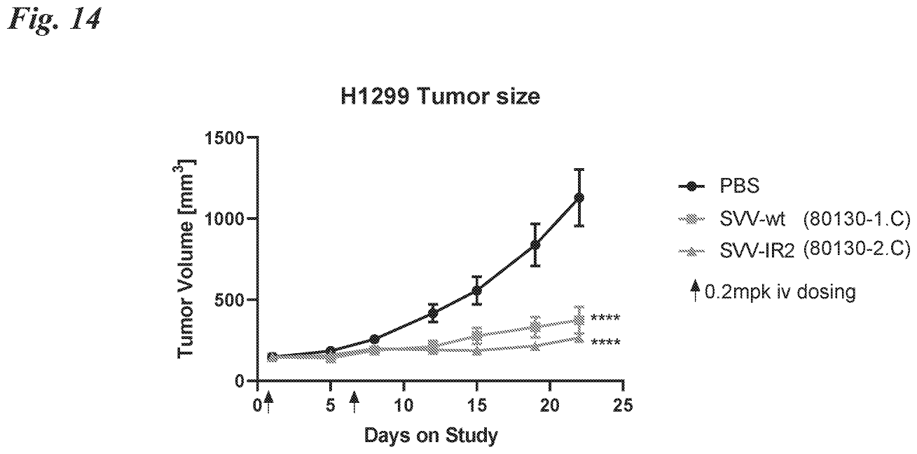

As shown in FIG. 14, mice treated with SVV-WT RNA or SVV-IRES2 RNA lipid nanoparticles exhibited significantly lower tumor burden compared to mice treated with PBS (two-way ANOVA, Tukey's multiple comparison test, ****p<0.0001). These results demonstrate that lipid nanoparticles containing SVV-WT or SVV-IRES2 RNA molecules exhibit anti-tumor activity in vivo, with SVV-IRES2 demonstrating the best anti-tumor effects.

### Example 13: Lipid Nanoparticles Containing SVV IRES2-Encoding RNA Molecules are Capable of Replicating in Neuroblastoma Tumors

Experiments were performed to determine the ability of SVV-WT and SVV-IRES2 RNA lipid nanoparticles to replicate in vivo.

SVV RNA lipid nanoparticle production, formulation and analysis is described in Example 2 and in Table 17 below.

TABLE 17
SVV RNA Lipid Nanoparticle Formulations

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 80130-1.C | SVV- | MB, pH 3.0 | SS-OC | Cholesterol | DSPC | DPG-PEG2K | 7 |
| | WT | | (49%) | (28.5%) | (22%) | (0.5%) | |
| 80130-2.C | SVV- | MB, pH 3.0 | SS-OC | Cholesterol | DSPC | DPG-PEG2K | 7 |
| | IRES2 | | (49%) | (28.5%) | (22%) | (0.5%) | |

The ability of SVV-WT and SVV-IRES2 lipid nanoparticles to inhibit tumor growth was evaluated using the N1E-115 xenograft model. Briefly, N1E-115 cells (5×10⁵ cells/0.1 mL in a 1:1 mixture of serum-free DPBS and Matrigel) were subcutaneously inoculated in the right flank of 8-week-old female A/J mice (Charles River Laboratories). When median tumor size reached approximately 150 mm³ (120-180 mm³range), mice were intravenously administered 0.4 mg/kg of SVV-WT (formulation ID: 80130-1.C) or SVV-IRES2 (formulation ID: 80130-2.C) RNA lipid nanoparticles (day 1). Tumors were harvested from mice 96 hours following lipid nanoparticle treatment and analyzed for the presence of minus-strand SVV RNA (a surrogate marker for replication SVV) using qRT-PCR.

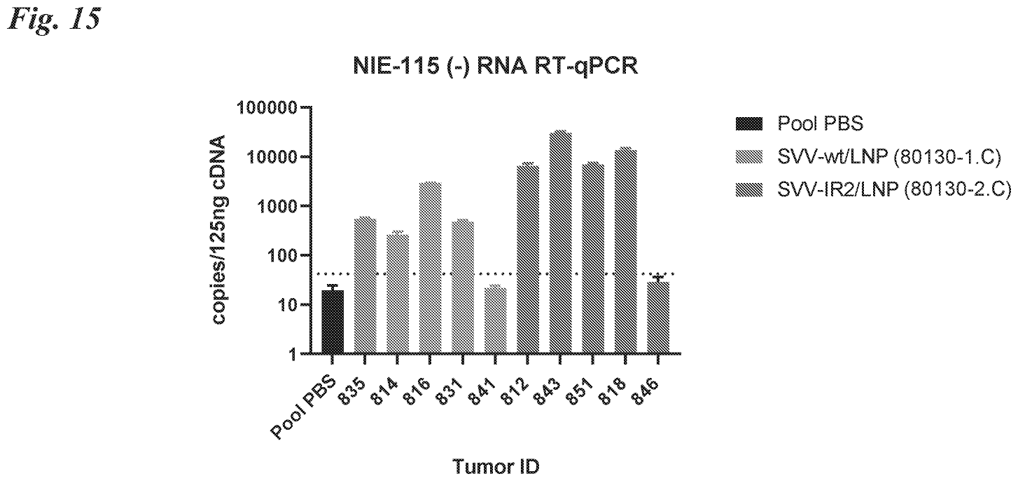

As shown in FIG. 15, mice administered SVV-WT or SVV-IRES2 RNA lipid nanoparticles showed 10-1000 fold higher levels of minus-strand SVV RNA compared to PBS controls. Notably, a majority of tumor tissue from mice treated with SVV-IRES2 lipid nanoparticles exhibited higher levels of SVV replication compared to mice administered SVV-WT RNA lipid nanoparticles. These results demonstrated that systemic administration of SVV-WT or SVV-IRES2 RNA lipid nanoparticles leads to SVV replication in neuroblastoma tumors in vivo.

### Example 14: Generation of Neutralizing Rabbit Polyclonal Antibody Against SVV

A rabbit polyclonal antibody was generated by immunizing rabbits with SVV virions. The presence of anti-SVV antibodies in the serum of immunized rabbits was confirmed by ELISA (data not shown).

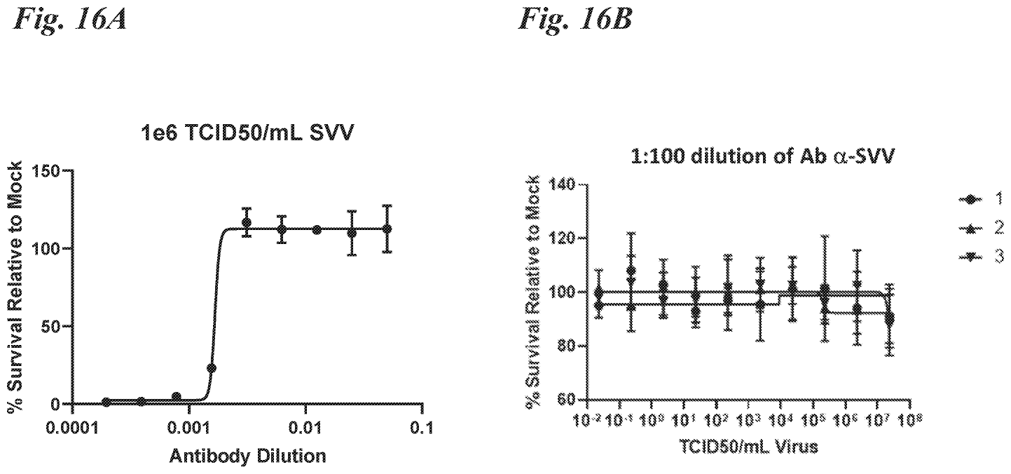

To determine whether anti-SVV antibodies could inhibit cell lysis, SVV virus (1×10⁶ TCID50/mL) and serial dilutions (1:2) of anti-SVV antibody were incubated on H446 cells. Lytic activity of H446 cells was calculated by CellTiter-Glo®. FIG. 16A shows that SVV is neutralized by rabbit polyclonal antibody at a dilution of up to 1:320.

To determine the effectiveness of a 1:100 dilution of rabbit anti-SVV polyclonal antibody, different concentrations of SVV virus and the diluted SVV antibody (1:100) were incubated on H446 cells. As shown in FIG. 16B, all doses of SVV virus tested, ranging from 10² to 10⁷ TCID50/mL, were neutralized by 1:100 dilution of anti-SVV antibody. The 1, 2, and 3 groups are biological replicates of the antibody dilutions.

### Example 15: SVV-RNA Lipid Nanoparticles Exhibit Anti-Tumor Activity in the Presence of Neutralizing Serum

SVV RNA lipid nanoparticles were tested in the presence of SVV neutralizing antibodies in vivo.

Generation and testing of anti-SVV polyclonal antibody is described in Example 14. SVV RNA lipid nanoparticle production, formulation, and analysis is described in Example 2 and Table 18 below.

TABLE 18
SVV RNA Lipid Nanoparticle Formulation

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 80139-1.C | SVV-WT | MB, pH 3.0 | SS-OC | Cholesterol | DSPC | DPG-PEG2K | 7 |
| | | | (49%) | (28.5%) | (22%) | (0.5%) | |

The ability of anti-SVV antibodies to inhibit tumor cell lysis induced by SVV RNA lipid nanoparticles was evaluated using the H1299 xenograft model, which is further described in Example 4. Briefly, nude mice bearing H1299 tumors (n=8 mice/group) were intravenously administrated 100 μL of SVV-RNA lipid nanoparticles (formulation ID: 80139-1.C) or SVV virions and either an intraperitoneal injection of naïve rabbit serum (negative control) or anti-SVV polyclonal antibody. Tumor-bearing mice received two doss antibody on day 0 and day 7 and two doses of SVV virions or SVV RNA lipid nanoparticles on day 1 ad day 8 of the study. Tumor volume was measured 3 times per week using electronic calipers. Treatment groups are shown in Table 19 below.

TABLE 19
Experimental Treatment Groups

| | | SVV-RNA LNP | Antibody | Antibody |
|---|---|---|---|---|
| Treatment | SVV Dose | Formulation | Dose | Lot |
| PBS + rabbit serum | — | — | 1.5 mg | 47-41-052316 |
| SVV-virus + rabbit serum | 1 × 10⁶ | — | 1.5 mg | 47-41-052316 |
| | TCID50/mL | | | |
| SVV-virus + aSVV antibody | 1 × 10⁶ | — | 1.5 mg | 1906102.IP |
| | TCID50/mL | | | |
| SVV-WT/LNP + rabbit serum | 1 mg/kg | 80139-1.C | 1.5 mg | 47-41-052316 |
| SVV-WT/LNP + aSVV antibody | 1 mg/kg | 80139-1.C | 1.5 mg | 1906102.IP |

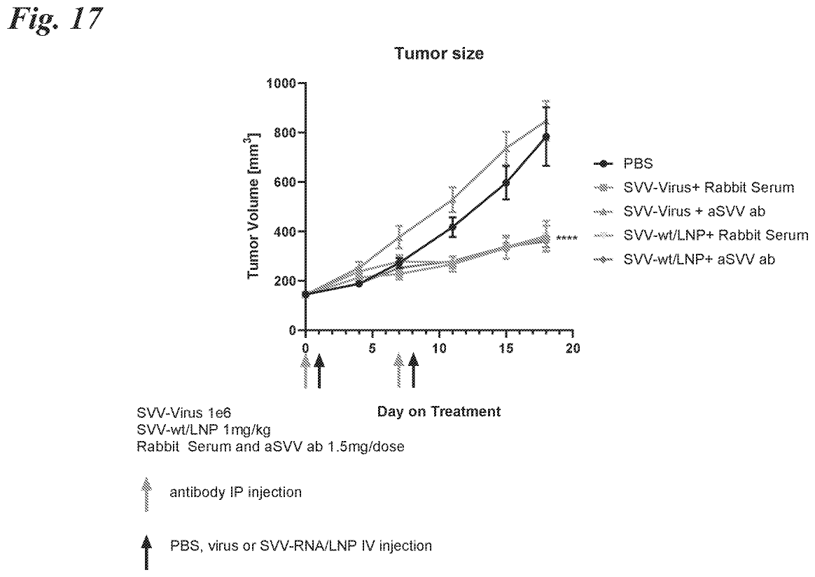

As shown in FIG. 17, SVV virion-treated mice immunized with naïve rabbit serum showed significant inhibition of tumor growth (two-way ANOVA, Tukey's multiple comparison test, ****p<0.0001). In contrast, administration of SVV-neutralizing antibodies to SVV virion-treated mice completely blocked the anti-tumor activity of SVV. Administration of SVV-neutralizing antibodies (or naïve rabbit serum) to mice treated with SVV RNA lipid nanoparticles did not affect the anti-tumor activity of SVV RNA lipid nanoparticles. SVV RNA lipid nanoparticle treatment significantly inhibited tumor growth in mice treated with SVV neutralizing serum (two-way ANOVA, Tukey's multiple comparison test, ****p<0.0001). Thus, unlike SVV virions, the anti-tumor activity of SVV RNA lipid nanoparticles is not affected by the presence of neutralizing antibodies in circulation.

### Example 16: In Vivo Efficacy of CVA21-Encoding RNA Lipid Nanoparticles in Melanoma

Experiments were performed to determine the ability of lipid nanoparticles comprising CVA21-encoding RNA molecules to produce infectious virus and inhibit melanoma tumor growth in vivo. CVA21 RNA lipid nanoparticle production, formulation, and analysis is described in Example 2 and in Table 20 below.

TABLE 20
Formulation of CVA21 Lipid Nanoparticles

| Formulation | | | Ionizable | Cholesterol | Helper | PEGylated | |
|---|---|---|---|---|---|---|---|
| ID | RNA | Buffer | lipid (%) | (%) | lipid (%) | lipid (%) | N:P |
| 70032-6.C | CVA21 | PB, pH 5.8 | MC3 | Cholesterol | DSPC | DSPE-PEG5K | 7 |
| | | | (49%) | (39.8%) | (11%) | (0.2%) | |

The ability of CVA21 RNA lipid nanoparticles to inhibit tumor growth was evaluated using the SK-MEL28 xenograft model. Briefly, SK-MEL28 cells (1×10⁶ cells/0.1 mL in a 1:1 mixture of serum-free PBS and Matrigel®) were subcutaneously inoculated in the right flank of 8-week-old female athymic nude mice (Charles River Laboratories). When median tumor size reached approximately 150 mm³ (120-180 mm³ range), mice were intratumorally administered either PBS or CVA21-encoding RNA formulated with Lipoafectime RNAiMAx (1 μg), or intravenously administered CVA21-encoding RNA lipid nanoparticles (formulation ID: 70032-6C, 5 μg). Mice received intratumoral treatments on days 1 and 5, or intravenous treatment on days 1, 6, 11, and 16. Tumor volume was measured 3 times per week using electronic calipers.

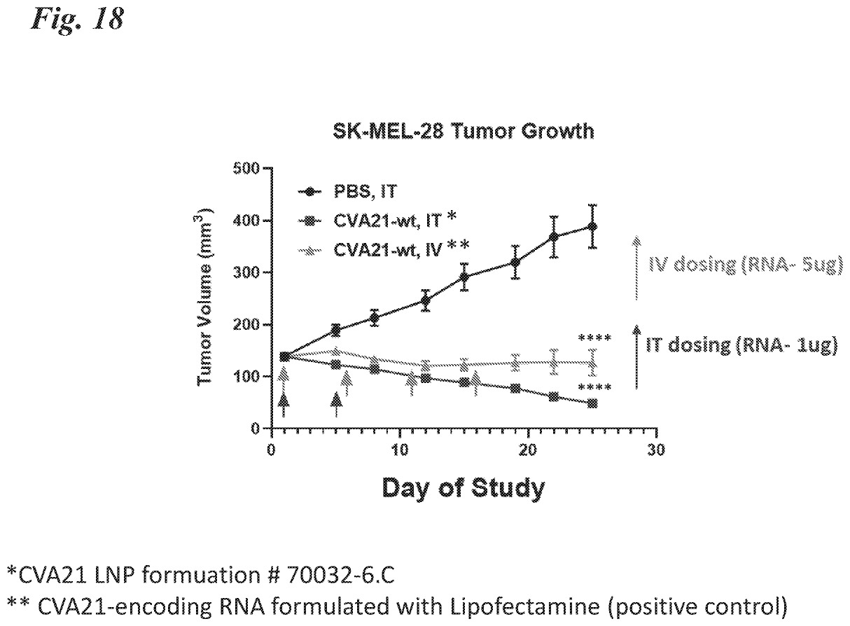

As shown in FIG. 18, intravenous treatment with LNPs comprising CVA21 RNA molecules or intratumoral treatment of CVA21-RNA molecules formulated with Lipofectamine prevented tumor growth in tumor-bearing mice compared to mice treated with PBS (two-way ANOVA, p<0.0.001). Collectively, these results suggest that lipid nanoparticles comprising CVA21 RNA molecules are an effective therapeutic strategy for the treatment of melanoma.

### Example 17 Strategies for Generation of Discrete 3′ Termini of SVV

As described above, the synthetic genomes described herein require discrete 3′ and 5′ ends native to the virus in order to produce a replication-competent and infective virus from the synthetic genome. The RNA transcripts produced by T7 RNA polymerase in vitro mammalian 5′ and 3′ UTRs and therefore do not contain the discrete, native ends required for production of an infectious ssRNA virus.

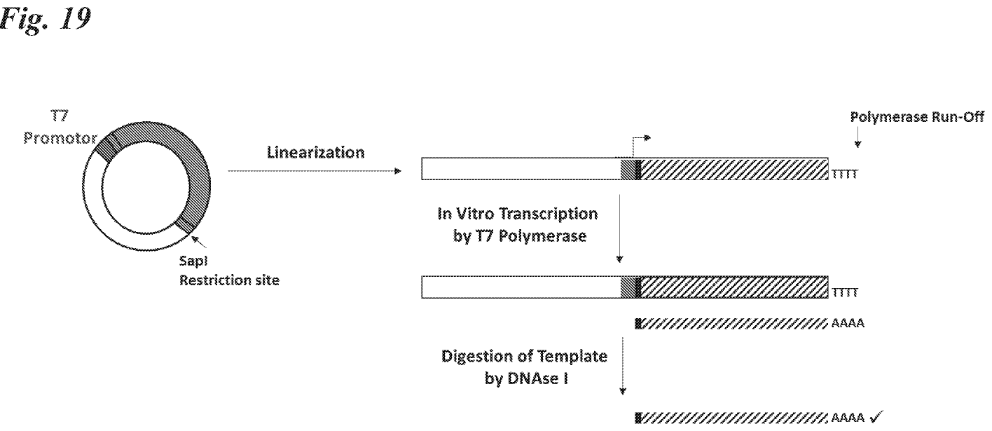

A strategy using 3′ restriction enzyme recognition sequences was employed to generate the discrete 3′ ends required for infectious SVV The SapI restriction recognition sequence was inserted at the 3′ end of the DNA template. SapI cleaves 5′ f its recognition site to generate a polythymidine run of the appropriate length to generate the discrete virus polyadenylation site native to the virus. This process is illustrated in FIG. 19.

### Example 18: An RNaseH Strategy for Generation of Discrete 5′ Termini of SVV

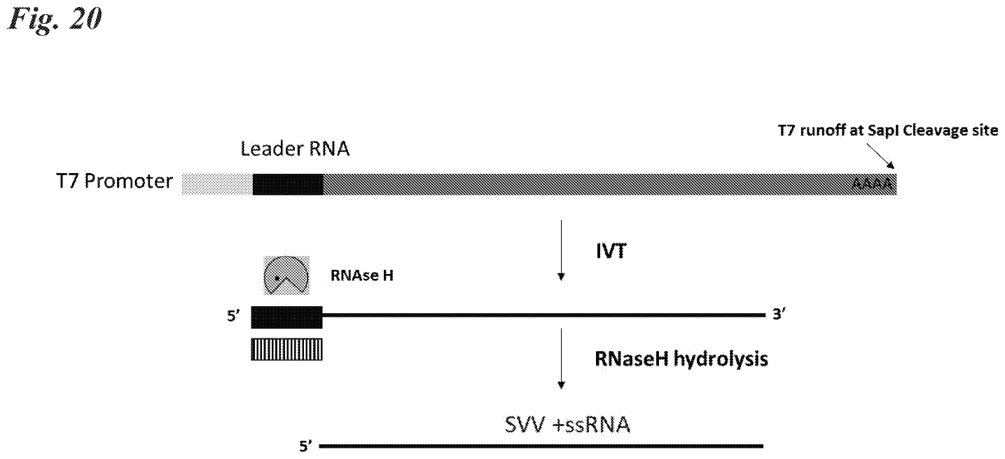

An RNAseH strategy was employed to generate the discrete 5′ termini native to SVV. T7 RNA polymerase requires a guanosine residue on the 5′ end. However, the 5′ terminus of SVV begins with a uridine residue. Thus the T7 leader must be removed to generate an authentic terminus for the virus. Depicted FIG. 20 is a diagram of the in vitro transcription (IVT) and 5° leader processing approach. The IVT template is depicted at the top and the resulting RNA transcript is illustrated in the middle. This SVV +ssRNA transcript is then annealed to a complementary dsDNA oligo (dashed box) and that portion is hydrolyzed with RNaseH. The final viral ssRNA product, with the correct 5′ terminus is shown at the bottom.

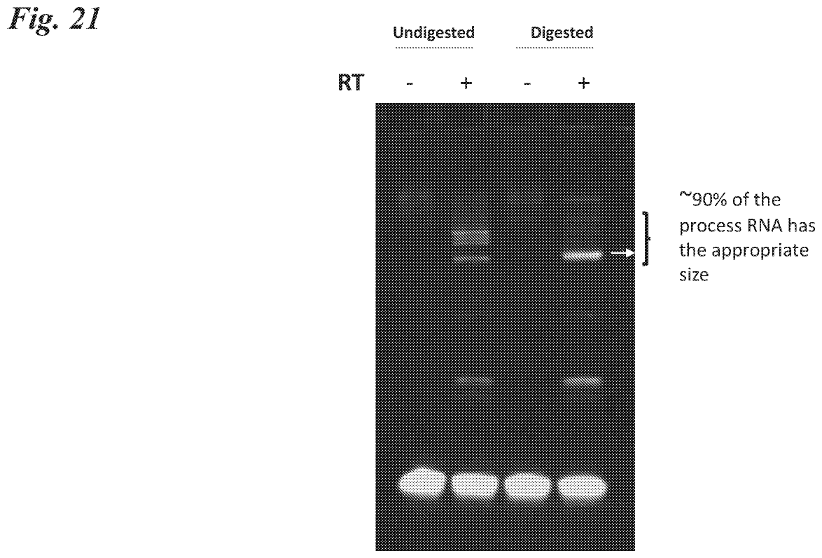

The correct processing of RNaseH engineered transcripts was assessed by a primer extension analysis. RNA transcripts were treated with RNaseH or left undigested. A complimentary fluorescent primer was then added and annealed to the digested and undigested samples and then extended b Superscript IV reverse transcriptase. The products were then resolved on a TBE UREA acrylamide gel. The expected bands were as follows: undigested≥150 bp and digested=100 bp. As shown in FIG. 21, this strategy results in ˜90% of the RNA processed correctly as illustrated by the increase in digested bands in the presence of RNaseH.

This strategy, in combination with the 3′ restriction enzyme strategy described in Example 19, produces a final synthetic SVV genome with the discrete 5′ and 3′ termini required for production of infectious SVV.

### Example 19: A Ribozyme Strategy for Generation of Discrete 5′ Termini of SVV

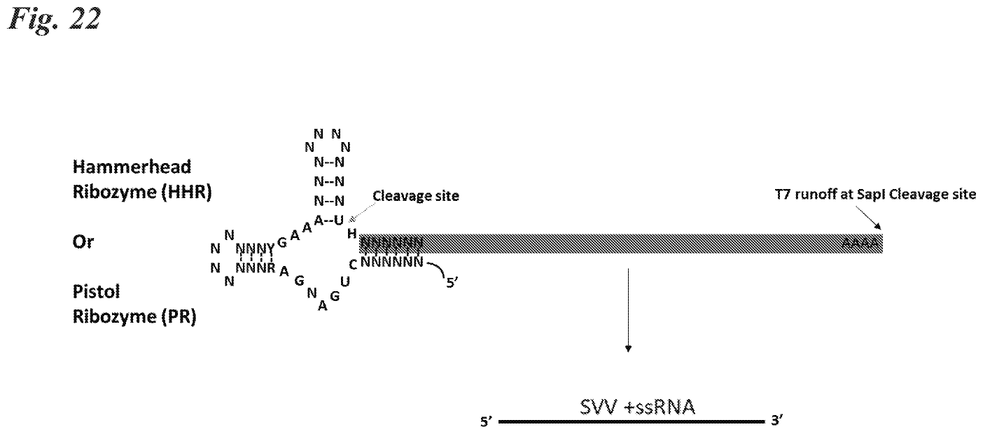

A ribozyme strategy was employed to generate the discrete 5′ termini native to SVV. A schematic of this approach is illustrated in FIG. 22, showing the design of ribozymes to cleave at the 5′ terminus of a picornavirus. The two ribozymes depicted are hammerhead and pistol ribozymes, however multiple other ribozymes could be adapted to cleave specifically in this context.

Modifications of the hammerhead and pistol ribozymes for implementation in this strategy are shown in FIG. 23 and FIG. 24, respectively. A structural model of a minimal hammerhead ribozyme (HHR) that anneals and cleaves the 5′ end of SVV is shown in FIG. 23A (this ribozyme cleaves the 5′ end at the site indicated by the arrow). A structural model of hammerhead ribozyme with a stabilized stem I for cleavage of the SVV 5′ terminus (STBL) is shown in FIG. 23B (this ribozyme cleaves the 5′ end at the site indicated by the arrow). FIG. 24A shows a schematic of Pistol ribozyme characteristics found in the wild (Pistol WT). FIG. 24B shows a Pistol ribozyme from P. polymyxa modeled by mFOLD with a tetraloop added to fuse the P3 strands. The nucleic acids in the dashed box were mutagenized to retain the fold of the ribozyme in the context of the viral sequence. The WT “GUC” sequence shown in the dashed box was mutated to “UCA” to generate Pistol 1 and the “GUC” sequence was mutated to “TTA” to generate Pistol 2.

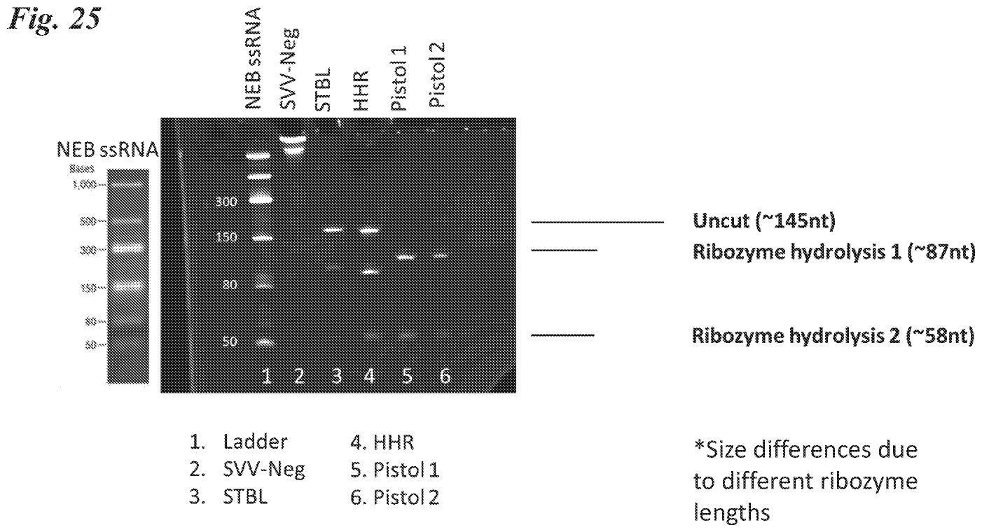

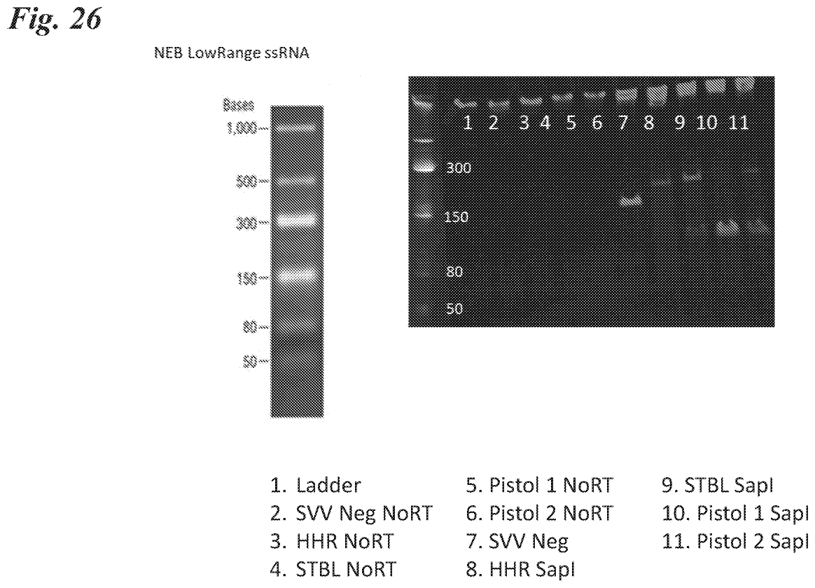

The ability of each of the 4 ribozymes (HHR, STBL, Pistol 1, and Pistol 2) to mediate cleavage during the IVT process was assessed. IVT templates were linearized with HpaI to yield 150 nt runoff in the uncut case, and 2 cleavage fragments of ˜90% and ˜60 nt in the case of ribozyme cleavage in order give a readout of ribozyme efficiency subsequent to the IVT process. As shown in FIG. 25, three bands were present in all reactions with the ribozyme constructs. The two hammerhead ribozymes cleave with ˜40-60% efficiency (STBL—lane 3 and HHR—lane 4). The Pistol 1 ribozyme in lane 5 had no visible uncut product on the gel. The Pistol 2 ribozyme in lane 6 had ˜5% uncleaved product visible on the gel. Similar results were obtained using primer extension analysis (FIG. 26). Primer extension products were resolved on a TBE UREA acrylamide gel. The expected bands were; undigested≥150 bp (See the reference band of SVV-Neg at 150 bp lane 7) and digested=80 bp. Pistol 1 results in complete cleavage evidenced by the absence of the upper band in lane 10.

These results demonstrate that all four of the ribozymes are able to mediate cleavage of the IVT transcript. However, the Pistol 1 and Pistol 2 ribozymes were more efficient, with Pistol 1 being the most efficient and mediating complete cleavage during the IVT process.

### Example 20: In Vitro Function of 5′ Ribozyme and 3′ Restriction Enzyme Engineered SVV Genomes

An in vitro assay was performed to assess the functionality of SVV genomes engineered with 5′ ribozyme sequences and 3′ SapI restriction enzyme recognition sites. The following IVT templates were used to generate synthetic SVV genomes:

(a) 5′ HHR sequences and 3′ SapI recognition sequences

(b) 5′ Pistol 1 sequences and 3′ SapI recognition sequences

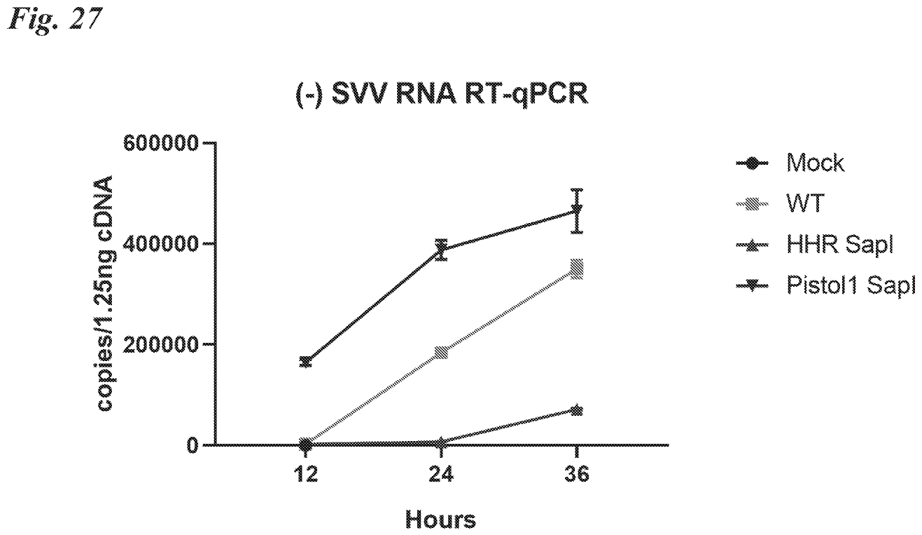

H1299 cells were transfected using Lipofectamine RNAiMax (Invitrogen) with 1 ug of IVT produced RNA. Total RNA was extracted from the NCI-H1299 cells at 12 hrs, 24 hrs, and 36 hrs using 250 uL QIAzol reagent from Qiagen. cDNA was produced from this RNA and analyzed with a minus-strand specific Taqman assay. NCI-H1299 cells were transfected with fixed amounts of the indicated IVT produced RNAs. An absolute qRT-PCR was performed on cDNA produced from this RNA over time. The early kinetics of SVV kickoff from H1299 cells is greatly enhanced in the Pistol1-SapI construct (FIG. 27).

### Example 21: In Vivo Efficacy of Pistol/SapI SVV RNA Lipid Nanoparticles on Tumor Growth

Experiments were performed to determine whether lipid nanoparticles containing SVV-Pistol/SapI RNA molecules could inhibit tumor growth in vivo.

SVV RNA lipid nanoparticle production, formulation, and analysis is described in Example 2 and in Table 21 below.

TABLE 21
SVV RNA Lipid Nanopartide Formulations

| Formulation | | | Ionizable | Cholesterol | Helper | PEGylated | |
|---|---|---|---|---|---|---|---|
| ID | SNA | Buffer | lipid (%) | (%) | lipid (%) | lipid (%) | N:P |
| 80130-1.C | SVV- | MB, pH 3.0 | SS-OC | Cholesterol | DSPC | DPG-PEG2K | 7 |
| | WT | | (49%) | (28.5%) | (22%) | (0.5%) | |
| | (HHR/HDV) | | | | | | |
| 80130-3.C | SVV- | MB, pH 3.0 | SS-LC | Cholesterol | DSPC | DPG-PEG2K | 7 |
| | WT | | (49%) | (28.5%) | (22%) | (0.5%) | |
| | (SapI/ | | | | | | |
| | Pistol) | | | | | | |

The ability of SVV-HHR-HDV and SVV-Pistol/SapI lipid nanoparticles to inhibit tumor growth was evaluated using the H1299 xenograft model, which is further described in Example 4. Briefly, 5×10⁶ NCI-H1299 cells were subcutaneously administered to the right flank of athymic nude mice. When median tumor size reached approximately 150 mm³, mice were intravenously administered 0.2 mg/kg of lipid nanoparticles containing SVV-HHR/HDV (formulation ID: 80130-1.C) or SVV-Pistol/SapI (formulation ID: 80130-3.C) RNA molecules (day 1). A subsequent dose of PBS or lipid nanoparticles was administered on day 8 of the study. Tumor volume was measured 3 times per week using electronic calipers.

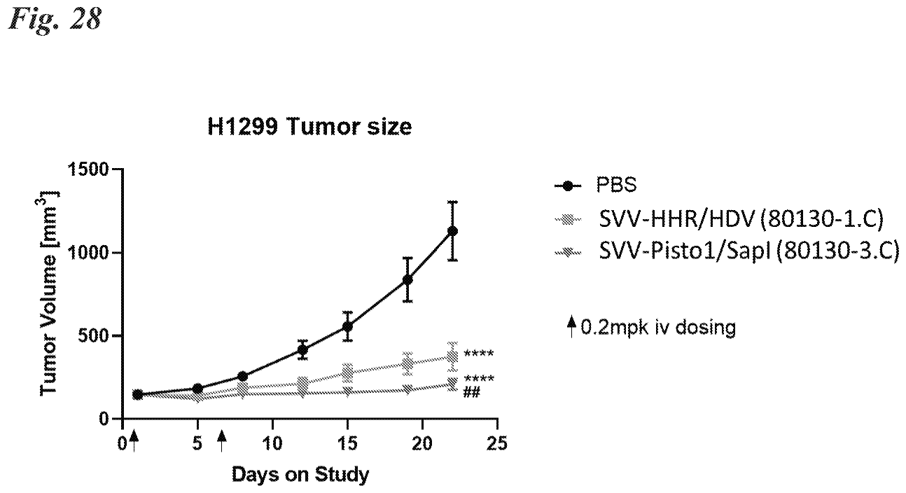

As shown in FIG. 28, mice treated with SVV-HHR-HDV RNA or SVV-Pistol/SapI RNA lipid nanoparticles significantly inhibited tumor growth compared to mice treated with PBS (two-way ANOVA, Tukey's multiple comparison test, ****p<0.0001). Moreover, treatment with SVV-Pistol/SapI RNA lipid nanoparticles was more effective at inhibiting tumor growth compared to SVV-HHR RNA lipid nanoparticles (two-way ANOVA, Tukey's multiple comparison test, #p<0.01).

### Example 22: In Vitro Synthesis of SVV-RNA with Modified-Ribonucleotides

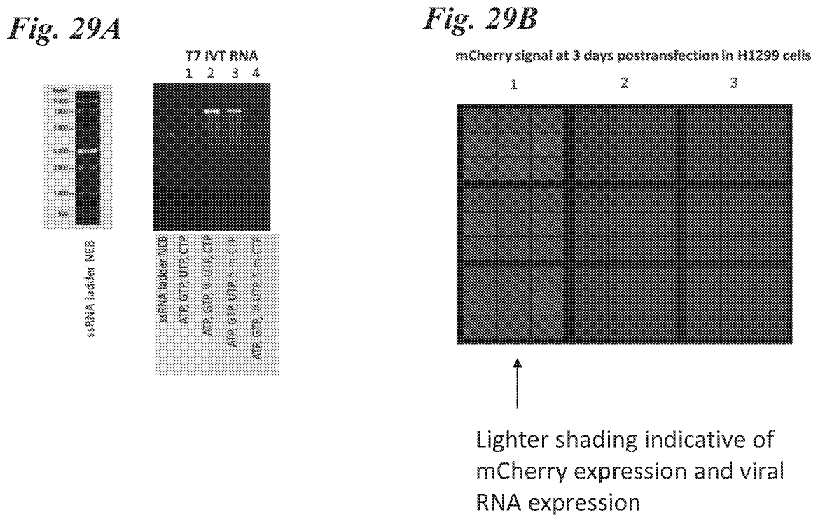

Experiments were performed to assess the possibility to in vitro synthesize the SVV-RNA with modified-ribonucleotide to enhance RNA stability. In vitro synthesis of SVV positive-sense RNA that encodes for mCherry (marker for gene expression and viral replication, SVV-mCherry) was conducted without or with modified-ribonucleotide (FIG. 29A). In vitro synthesized RNAs were formulated with RNAiMax (Invitrogen, Thermo Fisher, Waltham, Mass.) and transfected into NCI-H1299 human cancer lung cells for 4 h, after which cells were washed and fresh media was added for 72 hrs. Presence of red fluorescent protein mCherry (shown as lighter shading in FIG. 29B) was used as surrogate of viral replication.

As shown in FIG. 29A, synthesis of SVV-mCherry-RNA with ψ-UTP or 5-m-CTP was efficient. When both modified ribonucleotides were combined, no RNA was produced. This indicated that the presence of multiple modified ribonucleotide did not support the IVT of SVV-RNA. Transfection of SVV-ψ-UTP RNA or SVV-5-m-CTP RNA did not render viral replication (FIG. 29B). Viral RNA secondary structure is key to support viral replication, and in the case of SVV, the use of modified ribonucleotides which can alter the natural secondary conformation of the RNA, preventing viral replication.

### Example 23: Expression of Payload Molecules from SVV Genomes

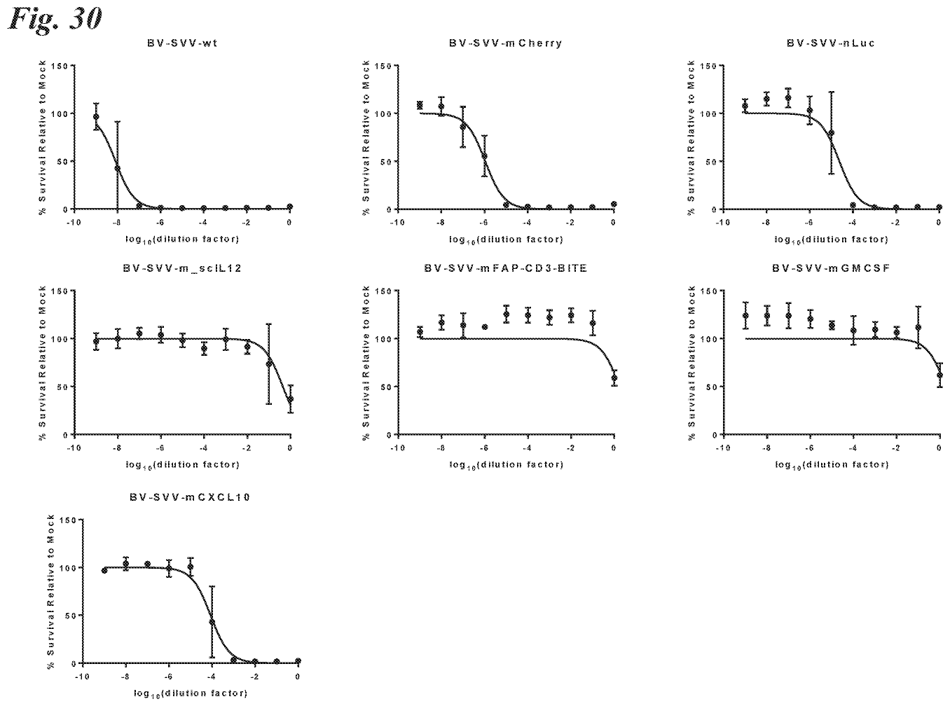

Experiments were performed to assess whether polynucleotides encoding payload molecules could be expressed from the SVV viral genomes. Expression cassettes for each of mCherry, nanoLuciferase, CXCL10, GM-CSF, and a FAP-CD3 BiTE were inserted into plasmids encoding the SVV genome. H1299 cells were transfected with 0.015 pmol of the plasmids and supernatants were harvested and filtered to collect viral particles. The filtered supernatants were transferred to H446 cultures and infectivity was assessed. As shown in FIG. 30, each of the payload engineered viruses were able to infect H446 cells, although to varying degrees of efficacy. The IC50s for each construct are provided below in Table 22. These data indicate the SVV can be engineered to express a variety of payload molecules to enhance the therapeutic efficacy of the virus.

TABLE 22
Plasmid length and IC50 for SVV-payload constructs

| | | Plasmid | | |
|---|---|---|---|---|
| | | Length | Payload | |
| Construct | SEQ ID | (bp) | (bp) | IC50 |
| NEP-BV-SVV-NEGATIVE | | 9729 | | >1e−01 |
| NEP-BV-SVV-NEG mCherry | | 10485 | 756 | >1e−01 |
| NEP-BV-SVV-WT | | 9865 | | 8.03E−09 |
| NEP-BV-SVV-mCherry | 19 | 10621 | 756 | 1.09E−06 |
| NEP-BV-SVV-m_scIL-12 | 22 | 11583 | 1718 | 4.43E−01 |
| NEP-BV-SVV-mCXCL10 | 24 | 10213 | 348 | 8.55E−05 |
| NEP-BV-SVV-mFAP-CD3 | 23 | 11460 | 1595 | >1e−01 |
| BITE | | | | |
| NEP-BV-SVV-nLuc | 20 | 10432 | 567 | 2.36E−05 |
| NEP-BV-SVV-mGM-CSF | 21 | 10288 | 423 | >1e−01 |

### Example 24: In Vivo Efficacy of SVV-Encoding a Payload RNA Lipid Nanoparticles on Tumor Growth and Payload Expression in Tumor Tissue

Experiments were performed to determine whether lipid nanoparticles containing SVV-payload RNA could inhibit tumor growth in vivo and assess payload expression in tumor tissue

SVV-Neg, SVV-WT and SVV-IL-36γ RNA (SEQ ID NO: 11) lipid nanoparticle production, formulation, and analysis is described in Example 2 and in Tables 23 and 24 below.

TABLE 23
Lipid compositions of formulations

| | | N/P | | Ionizable | | | Helper | | | |
|---|---|---|---|---|---|---|---|---|---|---|
| Formulation | RNA | ratio | Buffer | lipid | % | Chol % | lipid | % | PEG | % |
| 80116-3.C | SVV- | 7 | PB, pH | OC | 49% | 28.5% | DSPC | 22% | DPG- | 0.5% |
| | Neg | | 5.8 | | | | | | PEG2K | |
| 80116-4.C | SVV- | 7 | PB, pH | OC | 49% | 28.5% | DSPC | 22% | DPG- | 0.5% |
| | WT | | 5.8 | | | | | | PEG2K | |
| 80116-5.C | SVV- | 7 | PB, pH | OC | 49% | 28.5% | DSPC | 22% | DPG- | 0.5% |
| | IL- | | 5.8 | | | | | | PEG2K | |
| | 36γ | | | | | | | | | |

TABLE 24
Physical characteristics of formulations

| | | Z-Average | | | |
|---|---|---|---|---|---|
| Formulation | RNA | (d.nm) | PdI | ZP (mV) | % EE |
| 80116-3.C | SVV-Neg | 117 | 0.19 | −1.9 | 94 |
| 80116-4.C | SVV-WT | 112 | 0.16 | −1.4 | 93 |
| 80116-5.C | SVV-IL-36γ | 120 | 0.27 | 0.4 | 94 |

The ability of SVV-WT+SVV-Neg, and SVV-WT+SVV-IL-36γ lipid nanoparticles to inhibit tumor growth was evaluated using the H1299 xenograft model, which is further described in Example 4. Briefly, 5×10⁶ NCI-H1299 cells were subcutaneously administered to the right flank of athymic nude mice. When median tumor size reached approximately 150 mm³, mice were intravenously administered 0.2 mg/kg of lipid nanoparticles containing SVV-WT+SVV-Neg (formulation ID: 80116-3.C+80116-4.C mix at ratio 1:1) or SVV-WT+SVV-IL-36γ (formulation ID: 80116-4.C+80116-5.C mix at ratio 1:1) RNA molecules (day 1). A subsequent dose of PBS or lipid nanoparticles was administered on day 8 of the study. Tumor volume was measured 3 times per week using electronic calipers. Study was terminated on day 10 and tumor were collected for payload expression analysis.

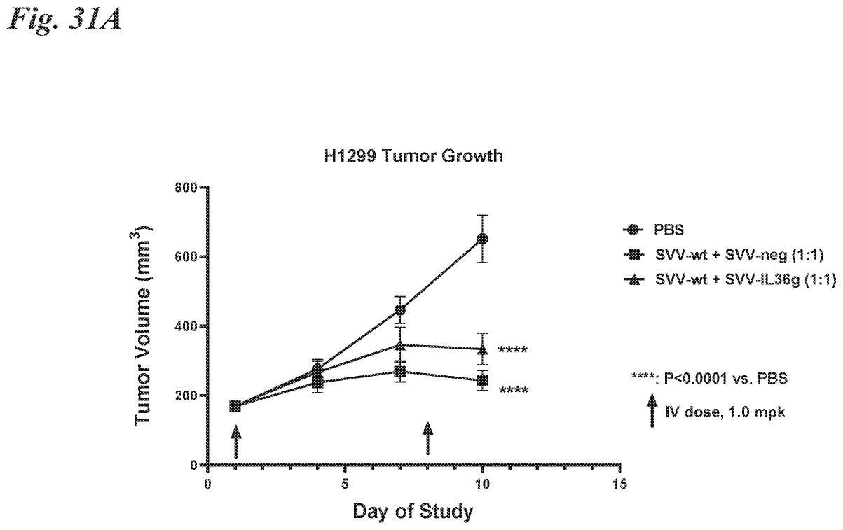

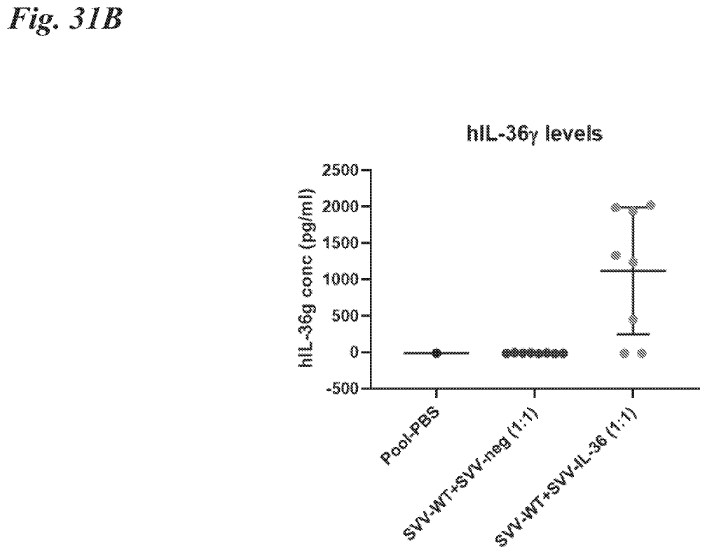

As shown in FIG. 31A, mice treated with SVV-WT+SVV-Neg or SVV-WT+SVV-IL-36γ RNA lipid nanoparticles exhibited significantly lower tumor burden compared to mice treated with PBS (two-way ANOVA, Tukey's multiple comparison test, ****p<0.0001). These results demonstrate that lipid nanoparticles containing SVV-WT+SVV-IL-36γ RNA molecules exhibit anti-tumor activity in vivo.

Tumor tissues collected at the end of the study were processed to generate tumor lysate. IL36γ levels were determined by Elisa (R&D System, DY2320-05). As shown in FIG. 31B, human IL-36γ was detected in mice treated with SVV-WT+SVV-IL36γ RNA lipid nanoparticles. These results demonstrate that lipid nanoparticles containing SVV-IL-36γ RNA molecules support the expression in tumor tissues of the transgene encoded in the viral genome.

### Example 25: Optimization of Coxsackievirus-Encoding RNA Molecules

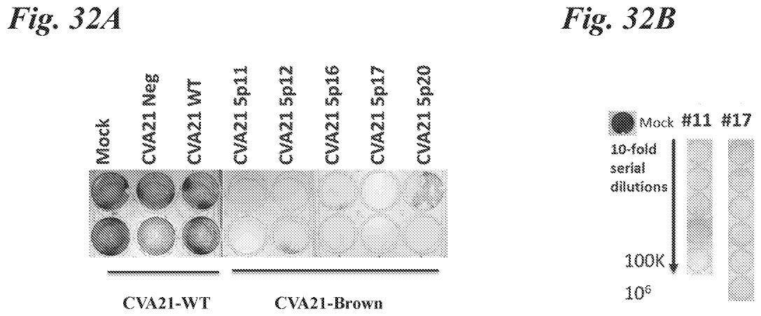

Experiments were performed to assess the ability to produce infectious Coxsackie Virus A21 (CVA21) from recombinant RNA molecules. Briefly, RNA polynucleotides comprising CVA21 viral genomes were generated by T7 transcription in vitro based on previously described CVA21 genome sequences (See Newcombe et al., Cellular receptor interactions of C-cluster human group A coxsackie viruses Journal of General Virology (2003), 84, 3041-3050. GenBank Accession No. AF465515). SK-MEL-28 cells were transfected with 1 μg of the CVA21 RNA constructs in Lipofectamine RNAiMax for 4 hours, at which point wells were washed and complete media was added to each well. After 48 hours, supernatants were removed from the SK-MEL-28 cultures and cells were stained with crystal violet to assess viral infectivity. As shown in FIG. 32A (left panel), RNA molecules comprising the Newcombe CVA21 sequences (CVA21 WT) did not produce active lytic virus (indicated by crystal violate staining of un-lysed SK-MEL-28 cells).

Surprisingly, alterations to the 5′ UTR were required for the production of infectious CVA21 from recombinant RNA molecule. As shown in FIG. 32A (right panel), incorporation of the 5′ UTR sequence described by Brown et al. (Journal of Virology, (2003)77:16, p. 8973-8984. GenBank Accession No. AF546702) into the CVA21 genome sequence described by Newcombe (CVA21-Brown) resulted in the production of infectious CVA21 virus and viral cell lysis, indicated by the lack of crystal violet staining across multiple independent clones. Supernatants from SK-MEL-28 cells transfected with two different CVA21-Brown clones were collected after 72 hours, and syringe filtered with 0:45 μM filter and serially diluted onto fresh SK-MEL-28 cells. After 48 hours, supernatants were removed from the SK-MEL-28 cultures and cells were stained with crystal violet to assess viral infectivity. As shown in FIG. 32B, CVA21 encoding RNA molecules comprising the Brown 5′ UTR sequence (UTR sequence—SEQ ID NO: 26, modified CVA21 sequence—SEQ ID NO: 27) resulted in the production and release of infectious CVA21 into the supernatant of transfected cells, indicated by the ability of the supernatants alone to mediate cell lysis.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

While preferred embodiments of the present disclosure have been shown and described herein; it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A lipid nanoparticle (LNP) comprising a synthetic RNA viral genome encoding an oncolytic virus.

2. The LNP of claim 1, wherein the oncolytic virus is a single-stranded RNA (ssRNA) virus.

3. The LNP of claim 1, wherein the oncolytic virus is a positive sense ((+)-sense) ssRNA virus.

4. The LNP of claim 3, wherein the (+)-sense ssRNA virus is selected from those listed in Table 1.

5. The LNP of claim 3, wherein the (+)-sense ssRNA virus is a Picornavirus.

6. The LNP of claim 5, wherein the Picornavirus is a Seneca. Valley Virus (SVV) or a Coxsackievirus.

7. The LNP of claim 6, wherein the SVV is an SVV-A selected from a wild type SVV-A (SEQ ID NO: 1), an S177A-SVVA mutant (SEQ ID NO: 2), an SVV-IR2 mutant (SEQ ID NO: 3), and an SVV-IR2-S177A mutant (SEQ ID NO: 4).

8. The LNP of claim 6, wherein the Coxsackievirus is selected from CVB3, CVA21, and CVA9,

9. The LNP of claim 6, wherein the Coxsackievirus is a modified CVA21 virus comprising SEQ ID NO: 27.

10. The LNP of any one of claims 1-9, wherein delivery of the LNP to a cell results in production of viral particles by the cell, and wherein the viral particles are infectious and lytic.

11. The LNP of any one of claims 1-10, wherein the synthetic RNA viral genome further comprises a heterologous polynucleotide encoding an exogenous payload protein

12. The LNP of any one of claims 1-10, further comprising a recombinant RNA molecule encoding an exogenous payload protein.

13. The LNP of claim 11 or 12, wherein the exogenous payload protein is a fluorescent protein, an enzymatic protein, a cytokine, a chemokine, an antigen-binding molecule capable of binding to a cell surface receptor, or a ligand for a cell-surface receptor.

14. The LNP of claim 13, wherein the cytokine is selected from IL-12, GM-CSF, 1L-18, IL-2, and IL-36γ.

15. The LNP of claim 13, wherein the ligand for a cell-surface receptor is Flt3 ligand or TNFSF14.

16. The LNP of claim 13, wherein the chemokine is selected from CXCL10, CCL4, CCL21, and CCL5.

17. The LNP of claim 13, wherein the antigen-binding molecule is capable of binding to and inhibiting an immune checkpoint receptor.

18. The LNP of claim 17, wherein the immune checkpoint receptor is PD-1.

19. The LNP of claim 13, wherein the antigen-binding molecule is capable of binding to a tumor antigen.

20. The LNP of claim 19, wherein the antigen binding molecule is a bispecific T cell engager molecule (BiTE) or a bispecific light T cell engager molecule (LiTE).

21. The LNP of claim 19 or 20, wherein the tumor antigen is DLL3 or EpCAM.

22. The LNP of any one of claims 1-18, wherein the synthetic RNA viral genome and/or the recombinant RNA molecule comprises a micro RNA (miRNA) target sequence (miR-TS) cassette, wherein the miR-TS cassette comprises one or more miRNA target sequences.

23. The LNP of claim 22, wherein the one or more miRNAs are selected from miR-124, miR-1, miR-143, miR-128, miR-219, miR-219a, miR-122, miR-204, miR-217, miR-137, and miR-126.

24. The LNP of claim 23, wherein the miR-TS cassette comprises one or more copies of a miR-124 target sequence, one or more copies of a miR-1 target sequence, and one or more copies of a miR-143 target sequence.

25. The LNP of claim 23, wherein the miR-TS cassette comprises one or more copies of a miR-128 target sequence, one or more copies of a miR-219a target sequence, and one or more copies of a miR-122 target sequence.

26. The LNP of claim 23, wherein the miR-TS cassette comprises one or more copies of a miR-128 target sequence, one or more copies of a miR-204 target sequence, and one or more copies of a miR-219 target sequence.

27. The LNP of claim 23, wherein the miR-TS cassette comprises one or more copies of a miR-217 target sequence, one or more copies of a miR-137 target sequence, and one or more copies of a miR-126 target sequence.

28. The LNP of any one of claims 1-27, wherein the LNP comprises a cationic lipid, one or more helper lipids and a phospholipid-polymer conjugate.

29. The LNP of claim 28, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

30. The LNP of claim 28 or 29, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol.

31. The LNP of claim 28, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

32. The LNP of any one of claims claim 28-30, wherein the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methypolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

33. The LNP of any one of claims 28-32, wherein the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

34. The LNP of claim 28, wherein the cationic lipid comprises COATSOME® SS-OC, wherein the one or more helper lipids comprise cholesterol (Chol) and DSPC, and wherein the phospholipid-polymer conjugate comprises DPG-PEG2000.

35. The LNP of claim 34, wherein the ratio of SS-OC:DSPC:Chol:DPG-PEG2K (as a percentage of total lipid content) is A:B:C:D, wherein A=40%-60%, B=10%-25%, C=20%-30%, and D=0%-3% and wherein A+B+C+D=100%.

36. The LNP of claim 34, wherein the ratio of SS-OC:DSPC:Chol:DPG-PEG2K (as a percentage of total lipid content) is A:B:C:D, wherein A=45%-50%, B=20%-25%, C=25%-30%, and D=0%-1% and wherein A+B+C+D=100%.

37. The LNP of claim 34, wherein the ratio of SS-OC:DSPC:Chol:DPG-PEG2K (as a percentage of total lipid content) is about 49:22:28.5:0.5.

38. The LNP of claim 34, wherein the ratio of SS-OC:DSPC:Chol:DPG-PEG2K (as a percentage of total lipid content) is A:B:C:D, wherein A=40%-60%, B=10%-30%, C=20%-45%, and D=0%-3% and wherein A+B+C+D=100%.

39. The LNP of claim 34, wherein the ratio of SS-OC:DSPC:Chol:DPG-PEG2K (as a percentage of total lipid content) is A:B:C:D, wherein A=40%-60%, B=10%-30%, C=25%-45%, and D=0%-3% and wherein A+B+C+D=100%.

40. The LNP of claim 34, wherein the ratio of SS-OC:DSPC:Chol:DPG-PEG2K (as a percentage of total lipid content) is A:B:C:D, wherein A=45%-55%, B=10%-20%, C=30%-40%, and D=1%-2% and wherein A+B+C+D=100%.

41. The LNP of claim 34, wherein the ratio of SS-OC:DSPC:Chol:DPG-PEG2K (as a percentage of total lipid content) is A:B:C:D, wherein A=45%-50%, B=10%-15%, C=35%-40%, and D=1%-2% and wherein A+B+C+D=100%.

42. The LNP of claim 34, wherein the ratio of SS-OC:DSPC:Chol:DPG-PEG2K (as a percentage of total lipid content) is 49:11:38.5:1.5.

43. The LNP of claim 34, wherein the ratio of SS-OC:DSPC:Chol:DPG-PEG2K (as a percentage of total lipid content) is about A:B:C:D, wherein A=45%-65%, B=5%-20%, C=20%-45%, and D=0%-3% and wherein A+B+C+D=100%.

44. The LNP of claim 34, wherein the ratio of SS-OC:DSPC:Chol:DPG-PEG2K (as a percentage of total lipid content) is about A:B:C:D, wherein A=50%-60%, B=5%-15%, C=30%-45%, and D=0%-3% and wherein A+B+C+D=100%.

45. The LNP of claim 34, wherein the ratio of SS-OC:DSPC:Chol:DPG-PEG2K (as a percentage of total lipid content) is about A:B:C:D, wherein A=55%-60%, B=5%-15%, C=30%-40%, and D=1%-2% and wherein A+B+C+D=100%.

46. The LNP of claim 34, wherein the ratio of SS-OC:DSPC:Chol:DPG-PEG2K (as a percentage of total lipid content) is about A:B:C:D, wherein A=55%-60%, B=5%-10%, C=30%-35%, and D=1%-2% and wherein A+B+C+D=100%.

47. The LNP of claim 34, wherein the ratio of SS-OC:DSPC:Chol:DPG-PEG2K (as a percentage of total lipid content) is 58:7:33.5:1.5.

48. The LNP of any one of claims 1-42, wherein the LNP comprises a lipid formulation selected from Table 5.

49. The LNP of any one of claims 1-48, wherein hyaluronan is conjugated to the surface of the LNP.

50. A therapeutic composition comprising a plurality of lipid nanoparticles according to any one of claims 1-49.

51. The therapeutic composition of claim 50, wherein the plurality of LNPs have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

52. The therapeutic composition of claim 50, wherein the plurality of LNPs have an average size of about 50 nm to about 120 nm.

53. The therapeutic composition of claim 50, wherein the plurality of LNPs have an average size of about 50 nm, 60 nm, 70 nm, 80 nm, 90 nm, 100 nm, 110 nm, or about 120 nm.

54. The therapeutic composition of claim 50, wherein the plurality of LNPs have an average size of about 100 nm.

55. The therapeutic composition of any one of claims 50-54, wherein the plurality of LNPs have an average zeta-potential of between about 40 mV to about −40 mV, about 20 mV to about −20 mV, about 10 mV to about −10 mV, about 5 mV to about −5 mV, or about 20 mV to about −40 mV.

56. The therapeutic composition of any one of claims 50-54, wherein the plurality of LNPs have an average zeta-potential of less than about −20 mV, less than about −30 mV, less than about −35 mV, or less than about −40 mV.

57. The therapeutic composition of claim 56, wherein the plurality of LNPs have an average zeta-potential of between about −50 mV to about −20 mV, about −40 mV to about −20 mV, or about −30 mV to about −20 mV.

58. The therapeutic composition of claim 56 or 57, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

59. The therapeutic composition of any one of claims 50-58, wherein administering the therapeutic composition to a subject delivers the recombinant RNA polynucleotide to a target cell of the subject, and wherein the recombinant RNA polynucleotide produces an infectious oncolytic virus capable of lysing the target cell of the subject.

60. The therapeutic composition of claim 59, wherein the composition is formulated for intravenous or intratumoral delivery.

61. The therapeutic composition of claim 59, wherein the target cell is a cancerous cell.

62. A method of inhibiting the growth of a cancerous tumor in a subject in need thereof comprising administering the therapeutic composition according to any one of claims 50-61 to the subject in need thereof, wherein administration of the composition inhibits the growth of the tumor.

63. The method of claim 62, wherein the administration is intratumoral or intravenous.

64. The method of claim 62 or 63, wherein the cancer is a lung cancer, a liver cancer, a prostate cancer, a bladder cancer, a pancreatic cancer, a gastric cancer, a breast cancer, a neuroblastoma, a rhabdomyosarcoma, a medullablastoma, or a melanoma.

65. The method of any one of claims 62-64, wherein the cancer is a neuroendocrine cancer.

66. A recombinant RNA molecule comprising a synthetic RNA viral genome encoding an oncolytic virus.

67. The recombinant RNA molecule of claim 66, wherein the encoded oncolytic virus is a single-stranded RNA (ssRNA) virus

68. The recombinant RNA molecule of claim 67, wherein the ssRNA virus is a positive sense ((+)-sense) or a negative-sense ((−)-sense) ssRNA virus.

69. The recombinant RNA molecule of claim 68, wherein the (+)-sense ssRNA virus is a Picornavirus.

70. The recombinant RNA molecule of claim 69, wherein the Picornavirus is a Seneca Valley Virus (SVV) or a Coxsackievirus.

71. The recombinant RNA molecule of claim 70, wherein the SVV is an SVV-A selected from a wild type SVV-A (SEQ ID NO: 1), an S177A-SVVA mutant (SEQ ID NO: 2), an SVV-IR2 mutant (SEQ ID NO: 3), or an SVV-IR2-S177A (SEQ ID NO: 4).

72. The recombinant RNA molecule of claim 70, wherein the Coxsackievirus is selected from CVB3, CVA21, and CVA9.

73. The recombinant RNA molecule of claim 70, wherein the Coxsackievirus is a modified CVA21. virus comprising SEQ ID NO: 27.

74. The recombinant RNA molecule of any one of claims 66-73, further comprising a micro RNA (miRNA) target sequence (miR-TS) cassette inserted into the polynucleotide sequence encoding the oncolytic virus, wherein the miR-TS cassette comprises one or more miRNA target sequences, and wherein expression of one or more of the corresponding miRNAs in a cell inhibits replication of the encoded virus in the cell.

75. The recombinant RNA molecule of claim 74, wherein the one or more miRNAs are selected from miR-124, miR-143, miR-128, miR-219, miR-219a, miR-122, miR-204, miR-217, miR-137, and miR-126.

76. The recombinant RNA molecule of claim 75, wherein the miR-TS cassette comprises one or more copies of a miR-124 target sequence, one or more copies of a miR-1 target sequence, and one or more copies of a miR-143 target sequence.

77. The recombinant RNA molecule of claim 75, wherein the miR-TS cassette comprises one or more copies of a miR-128 target sequence, one or more copies of a miR-219a target sequence, and one or more copies of a miR-122 target sequence.

78. The recombinant RNA molecule of claim 75, wherein the miR-TS cassette comprises one or more copies of a miR-128 target sequence, one or more copies of a miR-204 target sequence, and one or more copies of a miR-219 target sequence.

79. The recombinant RNA molecule of claim 75, wherein the miR-TS cassette comprises one or more copies of a miR-217 target sequence, one or more copies of a miR-137 target sequence, and one or more copies of a miR-126 target sequence.

80. The recombinant RNA molecule of any one of claims 66-79, wherein the recombinant RNA molecule is capable of producing a replication-competent oncolytic virus when introduced into a cell by a non-viral delivery vehicle.

81. The recombinant RNA molecule of claim 80, wherein the cell is a mammalian cell.

82. The recombinant RNA molecule of claim 81, wherein the cell is a mammalian cell present in a mammalian subject.

83. The recombinant RNA molecule of any one of claims 66-82, wherein the replication-competent virus is selected from the group consisting of coxsackie virus, polio virus, Seneca valley virus, lassa virus, murine leukemia virus, influenza A virus, influenza B virus, Newcastle disease virus, measles virus, sindbis virus, and a maraba virus.

84. The recombinant RNA molecule of claim 74, wherein the one or more miR-TS cassettes is incorporated into the 5′ untranslated region (UTR) or 3′ UTR of one or more essential viral genes.

85. The recombinant RNA molecule of claim 84, wherein the one or more miR-TS cassettes is incorporated into the 5′ untranslated region (UTR) or 3′ UTR of one or more non-essential genes.

86. The recombinant RNA molecule of any of claims 66-85, wherein the recombinant RNA molecule is inserted into a nucleic acid vector.

87. The recombinant RNA molecule of claim 141, wherein the nucleic acid vector is a replicon.

88. The recombinant RNA molecule of claims 66-87, wherein the synthetic RNA viral genome further comprises a heterologous polynucleotide encoding an exogenous payload protein

89. The recombinant RNA molecule of claim 88, wherein the exogenous payload protein is a fluorescent protein, an enzymatic protein, a cytokine, a chemokine, an antigen-binding molecule capable of binding to a cell surface receptor, or a ligand capable of binding to a cell surface receptor.

90. The recombinant RNA molecule of claim 89, wherein the cytokine is selected from IL-12, GM-CSF, IL-18, IL-2, and IL-36γ.

91. The recombinant RNA molecule of claim 89, wherein the ligand for a cell-surface receptor is Flt3 ligand or TNESF14.

92. The recombinant RNA molecule of claim 89, wherein the chemokine is selected from CXCL10, CCL4, CCL21, and CCL5.

93. The recombinant RNA molecule of claim 89, wherein the antigen-binding molecule is capable of binding to and inhibiting an immune checkpoint receptor.

94. The recombinant RNA molecule of claim 93, wherein the immune checkpoint receptor is PD-1.

95. The recombinant RNA molecule of claim 89, wherein the antigen-binding molecule is capable of binding to a tumor antigen.

96. The recombinant RNA molecule of claim 95, wherein the antigen binding molecule is a bispecific T cell engager molecule (BiTE) or a bispecific light T cell engager molecule (LiTE).

97. The recombinant RNA molecule of claim 95 or 96, wherein the tumor antigen is DLL3 or EpCAM.

98. A recombinant DNA molecule comprising from 5′ to 3′, a promoter sequence, a 5′ junctional cleavage sequence, a polynucleotide sequence encoding the recombinant RNA molecule of any one of claims 66-97, and a 3′ junctional cleavage sequence.

99. The recombinant DNA molecule of claim 98, wherein the promoter sequence is a T7 promoter sequence.

100. The recombinant DNA molecule of claim 98 or 99, wherein the 5′ junctional cleavage sequence is a ribozyme sequence and the 3′ junctional cleavage sequence is a ribozyme sequence.

101. The recombinant DNA molecule of claim 100, wherein the 5′ ribozyme sequence is a hammerhead ribozyme sequence and wherein the 3′ ribozyme sequence is a hepatitis delta virus ribozyme sequence.

102. The recombinant DNA molecule of claim 98 or 99, wherein the 5′ junctional cleavage sequence is a ribozyme sequence and the 3′ junctional cleavage sequence is a restriction enzyme recognition sequence.

103. The recombinant DNA molecule of claim 102, wherein the 5′ ribozyme sequence is a hammerhead ribozyme sequence, a Pistol ribozyme sequence, or a modified Pistol ribozyme sequence.

104. The recombinant DNA molecule of claim 102 or 103, wherein 3′ restriction enzyme recognition sequence is a Type IIS restriction enzyme recognition sequence.

105. The recombinant DNA molecule of claim 104, wherein the Type IIS recognition sequence is a SapI recognition sequence.

106. The recombinant DNA molecule of claim 98 or 99, wherein the 5′ junctional cleavage sequence is an RNAseH primer binding sequence and the 3′ junctional cleavage sequence is a restriction enzyme recognition sequence.

107. A method of producing the recombinant RNA molecule of any one of claims 66-97, comprising in vitro transcription of the DNA molecule of any one of claims 98-106 and purification of the resulting recombinant RNA molecule.

108. The method of claim 107, wherein the recombinant RNA molecule comprises 5′ and 3′ ends that are native to the oncolytic virus encoded by the synthetic RNA viral genome.

109. A composition comprising an effective amount of the recombinant RNA molecule of any one of claims 66-97, and a carrier suitable for administration to a mammalian subject.

110. A particle comprising the recombinant RNA molecule of any one of claims 66-97.

111. The particle of claim 110, wherein the particle is biodegradable.

112. The particle of claim 111, wherein the particle is selected from the group consisting of a nanoparticle, an exosome, a liposome, and a lipoplex.

113. The particle of claim 112, wherein the exosome is a modified exosome derived from an intact exosome or an empty exosome.

114. The particle of claim 112, wherein the nanoparticle is a lipid nanoparticle (LNP) comprising cationic lipid, one or more helper lipids and a phospholipid-polymer conjugate.

115. The particle of claim 114, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS-OC, COATSOME® SS-OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

116. The particle of claim 114 or 115, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); 1,2-dioleoyl-sn-glycero-3-phosphoethanolamine (DOPE); and cholesterol.

117. The particle of claim 114, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

118. The particle of claim any one of claims 114-116, wherein the phospholipid-polymer conjugate is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethyene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

119. The particle of any one of claims 114-118, wherein the phospholipid-polymer conjugate is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

120. The particle of claim 114, wherein the cationic lipid comprises COATSOME® SS-OC, wherein the one or more helper lipids comprise cholesterol (Chol) and DSPC, and wherein the phospholipid-polymer conjugate comprises DPG-PEG2000.

121. The particle of claim 120, wherein the ratio of SS-OC:DSPC:Chol:DPG-PEG2K (as a percentage of total lipid content) is A:B:C:D, wherein A=40%-60%, B=10%-25%, C=20%-30%, and D=0%-3% and wherein A+B+C+D=100%.

122. The particle of claim 120, wherein the ratio of SS-OC:DSPC:Chol:DPG-PEG2K (as a percentage of total lipid content) is A:B:C:D, wherein A=45%-50%, B=20%-25%, C=25%-30%, and D=0%-1% and wherein A+B+C+D=100%.

123. The particle of claim 120, wherein the ratio of SS-OC:DSPC:Chol:DPG-PEG2K (as a percentage of total lipid content) is about 49:22:28.5:0.5.

124. The particle of claim 120, wherein the ratio of SS-OC:DSPC:Chol:DPG-PEG2K (as a percentage of total lipid content) is A:B:C:D, wherein A=40%-60%, B=10%-30%, C=20%-45%, and D=0%-3% and wherein A+B+C+D=100%.

125. The particle of claim 120, wherein the ratio of SS-OC:DSPC:Chol:DPG-PEG2K (as a percentage of total lipid content) is A:B:C:D, wherein A=40%-60%, B=10%-30%, C=25%-45%, and D=0%-3% and wherein A+B+C+D=100%.

126. The particle of claim 120, wherein the ratio of SS-OC:DSPC:Chol:DPG-PEG2K (as a percentage of total lipid content) is A:B:C:D, wherein A=45%-55%, B=10%-20%, C=30%-40%, and D=1%-2% and wherein A+B+C+D=100%.

127. The particle of claim 120, wherein the ratio of SS-OC:DSPC:Chol:DPG-PEG2K (as a percentage of total lipid content) is A:B:C:D, wherein A=45%-50%, B=10%-15%, C=35%-40%, and D=1%-2% and wherein A+B+C+D=100%.

128. The particle of claim 120, wherein the ratio of SS-OC:DSPC:Chol:DPG-PEG2K (as a percentage of total lipid content) is 49:11:38.5:1.5.

129. The particle of claim 120, wherein the ratio of SS-OC:DSPC:Chol:DPG-PEG2K (as a percentage of total lipid content) is about A:B:C:D, wherein A=45%-65%, B=5%-20%, C=20%-45%, and D=0%-3% and wherein A+B+C+D=100%.

130. The particle of claim 120, wherein the ratio of SS-OC:DSPC:Chol:DPG-PEG2K (as a percentage of total lipid content) is about A:B:C:D, wherein A=50%-60%, B=5%-15%, C=30%-45%, and D=0%-3% and wherein A+B+C+D=100%.

131. The particle of claim 120, wherein the ratio of SS-OC:DSPC:Chol:DPG-PEG2K (as a percentage of total lipid content) is about A:B:C:D, wherein A=55%-60%, B=5%-15%, C=30%-40%, and D=1%-2% and wherein A+B+C+D=100%.

132. The particle of claim 120, wherein the ratio of SS-OC:DSPC:Chol:DPG-PEG2K (as a percentage of total lipid content) is about A:B:C:D, wherein A=55%-60%, B =5%-10%, C=30%-35%, and D=1%-2% and wherein A+B+C+D=100%.

133. The particle of claim 120, wherein the ratio of SS-OC:DSPC:Chol:DPG-PEG2K (as a percentage of total lipid content) is 58:7:33.5:1.5.

134. The particle of any one of claims 110-133, wherein the LNP comprises a lipid formulation selected from Table 5.

135. The particle of claim 114, wherein the cationic lipid is 1,2-dioleoyl-3-trimethylammonium-propane (DOTAP), and wherein the neutral lipid is 1,2-Dilauroyl-sn-glycero-3-phosphoethanolamine (DLPE) or 1,2-Dioleoyl-sn-glycero-3-phosphoethanolamine (DOPE).

136. The particle of claim 114 or 135, further comprising a phospholipid-polymer conjugate, wherein the phospholipid-polymer conjugate is 1,2-Distearoyl-sn-glycero-3-phosphoethanolamine-Poly(ethylene glycol) (DSPE-PEG) or 1,2-distearoyl-sn-glycero-3-phosphoethanolamine-N-[amino(polyethylene glycol)] (DSPE-PEG-amine).

137. The particle of any one of claims 110-136, wherein hyaluronan is conjugated to the surface of the LNP.

138. The particle of any one of claims 110-136, further comprising a second recombinant RNA molecule encoding a payload molecule.

139. The particle of claim 138, wherein the second recombinant RNA molecule is a replicon.

140. A therapeutic composition comprising a plurality of lipid nanoparticles according to any one of claims 114-139.

141. The therapeutic composition of claim 140, wherein the plurality of LNPs have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

142. The therapeutic composition of claim 140 wherein the plurality of LNPs have an average size of about 50 nm to about 120 nm.

143. The therapeutic composition of claim 140 wherein the plurality of LNPs have an average size of about 50 nm, 60 nm, 70 nm, 80 nm, 90 nm, 100 nm, 110 nm, or about 120 nm.

144. The therapeutic composition of claim 140 wherein the plurality of LNPs have an average size of about 100 nm.

145. The therapeutic composition of any one of claims 140-144, wherein the plurality of LNPs have an average zeta-potential of between about 20 mV to about −20 mV, about 10 mV to about −10 mV, about 5 mV to about −5 mV, or about 20 mV to about −40 mV.

146. The therapeutic composition of any one of claims 140-144, wherein the plurality of LNPs have an average zeta-potential of less than about −20 mV, less than about −30 mV, less than about −35 mV, or less than about −40 mV.

147. The therapeutic composition of claim 146, wherein the plurality of LNPs have an average zeta-potential of between about −50 mV to about −20 mV, about −40 mV to about −20 mV, or about −30 mV to about −20 mV.

148. The therapeutic composition of claim 141 or 146, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

149. The therapeutic composition of any one of claims 140-148, wherein delivery of the composition to a subject delivers the encapsulated recombinant RNA molecule to a target cell, and wherein the encapsulated recombinant RNA molecule produces an infectious virus capable of lysing the target cell.

150. The therapeutic composition of claim 149, wherein the composition is formulated for intravenous or intratumoral delivery.

151. The therapeutic composition of claim 150, wherein the target cell is a cancerous cell.

152. An inorganic particle comprising the recombinant polynucleotide of any one of claims 66-97.

153. The inorganic particle of claim 152, wherein the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP).

154. The inorganic particle of claim 150, further comprising a second recombinant RNA molecule encoding a payload molecule.

155. The particle of claim 154, wherein the second recombinant RNA molecule is a replicon.

156. A composition comprising the inorganic particle of any one of claims 152-155, wherein the average diameter of the particles is less than about 500 nm, is between about 50 nm and 500 nm, is between about 250 nm and about 500 nm, or is about 350 nm.

157. A method of killing a cancerous cell comprising exposing the cancerous cell to the particle of any one of claim 1-49, 110-139, or 152-155, the recombinant RNA molecule of any one of claims 66-97, or compositions thereof, under conditions sufficient for the intracellular delivery of the particle to said cancerous cell, wherein the replication-competent virus produced by the encapsulated polynucleotide results in killing of the cancerous cell.

158. The method of claim 157, wherein the replication-competent virus is not produced in non-cancerous cells.

159. The method of claim 157 or 158, wherein the method is performed in vivo, in vitro, or ex vivo.

160. A method of treating a cancer in a subject comprising administering to a subject suffering from the cancer an effective amount of the particle of any one of claim 1-49, 110-139, or 152-155, the recombinant RNA molecule of any one of claims 66-97, or compositions thereof.

161. The method of claim 160, wherein the particle or composition thereof is administered intravenously, intranasally, as an inhalant, or is injected directly into a tumor.

162. The method of claim 160 or 161, wherein the particle or composition thereof is administered to the subject repeatedly.

163. The method of any of claims 160-162, wherein the subject is a mouse, a rat, a rabbit, a cat, a dog, a horse, a non-human primate, or a human.

164. The method of any of claims 160-163, wherein the cancer is selected from lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL).

165. The method of claim 164, wherein the lung cancer is small cell lung cancer or non-small cell lung cancer.

166. The method of claim 164, wherein the liver cancer is hepatocellular carcinoma (HCC).

167. The method of claim 164, wherein the prostate cancer is treatment-emergent neuroendocrine prostate cancer.

168. The method of any of claims 150-167, wherein the cancer is a neuroendocrine cancer.
